(19) United States
(12) Patent Application Publication
(10) Pub. No.: US 2024/0173420 A1
(43) Pub. Date: May 30, 2024

(54) OCULAR DELIVERY OF OLIGONUCLEOTIDES

(71) Applicant: UNIVERSITY OF MASSACHUSETTS, Boston, MA (US)

(72) Inventors: Claudio Punzo, Brookline, MA (US); Anastasia Khvorova, Westborough, MA (US); Dimas Echeverria Moreno, Worcester, MA (US); Annabelle Biscans, Gothenburg (SE); Julia F. Alterman, Westborough, MA (US); Matthew Hassler, Boston, MA (US); Shun-Yun Cheng, Westborough, MA (US)

(21) Appl. No.: 18/375,206

(22) Filed: Sep. 29, 2023

Related U.S. Application Data
(60) Provisional application No. 63/412,051, filed on Sep. 30, 2022.

(51) Int. Cl. A61K 47/54; A61K 9/00

(52) U.S. Cl. CPC A61K 47/554; A61K 9/0048; A61K 47/542; A61K 47/543; A61K 47/545; A61K 47/548

(57) ABSTRACT

Provided herein are conjugated oligonucleotides that are characterized by efficient and specific eye distribution.

### RELATED APPLICATIONS

The present invention claims the benefit of U.S. Provisional Patent Application Ser. No. 63/412,051, filed Sep. 30, 2022, the content of which is incorporated herein by reference in its entirety for all purposes.

### TECHNICAL FIELD

This disclosure relates to oligonucleotide conjugates and branched oligonucleotides for delivery to the eye.

### BACKGROUND

Ocular diseases are caused by genetic and non-genetic risk factors. Some of these diseases have a clear underlying genetic etiology (mutation) that can be passed on in a dominant, recessive or X-liked inheritance pattern. Such inherited retinal dystrophies are caused by more than 250 genes. These include for example dominant mutations that cause dominant Retinitis Pigmentosa, such as the P23H mutation in the rhodopsin gene, which is the most widespread dominant mutation among individuals suffering from dominant Retinitis Pigmentosa. Ocular disease of unclear etiology include disease such as age-related macular degeneration, diabetic retinopathy, and glaucoma. While some of these diseases are caused by environmental risk factors there are also many genetic risk factors that have been identified that are believed contribute to disease progression. Interestingly, there are also several noncoding RNA sequences that have been identified to contribute to various diseases. Thus, there is an unmet need to efficiently regulate gene expression in the eye to treat various eye disease. Described herein is a method to downregulate the expression of disease-causing genes in the eye that either directly or indirectly contribute to a pathology.

Oligonucleotides such as small interfering RNA (siRNA) molecules have been used to regulate gene expression levels across different organs. Their implementation in the eye, however, has been hampered by low permeability of the siRNA molecule into various cell types, stability of the siRNA and longevity of the knockdown effect. This is of particular importance in the eye as repeat injections on a bi-weekly or monthly base constitute a burden for patients and care providers and increase the risk of ocular complications. Described herein is an oligonucleotide platform in which oligonucleotides (e.g., siRNA molecules) have been chemically stabilized for prolonged gene knockdown and modified in their configuration or attachments for improved cell entry into different cell types of the retina.

### SUMMARY

Provided herein are methods of delivering oligonucleotide conjugates and branched oligonucleotides to the eye, and in particular, specific eye cells. The oligonucleotide conjugates and branched oligonucleotides are capable of efficient gene knockdown in the eye. Several different functional moieties and branched oligonucleotides demonstrated eye cell specific delivery upon administration.

The oligonucleotide conjugates and branched oligonucleotides described herein promote simple, efficient, non-toxic delivery of oligonucleotides (e.g., siRNA), and promote potent silencing of therapeutic targets in a range of eye cell types in vivo.

In one aspect, the disclosure provides a method for delivering an oligonucleotide conjugate to an eye of a subject, the method comprising administering the oligonucleotide conjugate to the subject, wherein the oligonucleotide conjugate comprises: i) an oligonucleotide comprising a 5′ and a 3′ end and complementarity to a target nucleic acid; and ii) a functional moiety linked to the oligonucleotide, wherein the functional moiety comprises any one of triple amine, retinoic acid, docosahexaenoic acid (DHA), docosanoic acid (DCA), α-tocopheryl succinate, or lithocholic acid (LA).

In another aspect, the disclosure provides a method for delivering a branched oligonucleotide to an eye of a subject, the method comprising administering the branched oligonucleotide to the subject, wherein the branched oligonucleotide comprises two or more oligonucleotides, each oligonucleotide comprising a 5′ and a 3′ end and complementarity to a target nucleic acid. In certain embodiments, one or more of the oligonucleotides of the branched oligonucleotide further comprises a functional moiety linked to the oligonucleotide, wherein the functional moiety comprises any one of triple amine, retinoic acid, DHA, DCA, α-tocopheryl succinate, or LA.

In certain embodiments, two DHA functional moieties are linked to the oligonucleotide.

In certain embodiments, the oligonucleotide comprises an antisense oligonucleotide or an siRNA.

In certain embodiments, the siRNA comprises a sense strand and an antisense strand. In certain embodiments, the antisense strand comprises about 15 nucleotides to 25 nucleotides in length. In certain embodiments, the sense strand comprises about 15 nucleotides to 25 nucleotides in length. In certain embodiments, the antisense strand is 20 nucleotides in length, 21 nucleotides in length, or 22 nucleotides in length. In certain embodiments, the sense strand is 15 nucleotides in length, 16 nucleotides in length, 18 nucleotides in length, or 20 nucleotides in length.

In certain embodiments, the siRNA comprises a double-stranded region of 15 base pairs to 20 base pairs. In certain embodiments, the siRNA comprises a double-stranded region of 15 base pairs, 16 base pairs, 18 base pairs, or 20 base pairs.

In certain embodiments, the siRNA comprises at least one blunt-end.

In certain embodiments, the siRNA comprises at least one single stranded nucleotide overhang. In certain embodiments, the siRNA comprises about a 2-nucleotide to 5-nucleotide single stranded nucleotide overhang. In certain embodiments, the siRNA comprises a 2-nucleotide single stranded nucleotide overhang or a 5-nucleotide single stranded nucleotide overhang.

In certain embodiments, the siRNA comprises naturally occurring nucleotides.

In certain embodiments, the siRNA comprises at least one modified nucleotide. In certain embodiments, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the siRNA comprises at least one modified internucleotide linkage. In certain embodiments, the modified internucleotide linkage comprises a phosphorothioate internucleotide linkage. In certain embodiments, the siRNA comprises 4-16 phosphorothioate internucleotide linkages. In certain embodiments, the siRNA comprises 8-13 phosphorothioate internucleotide linkages.

In certain embodiments, the siRNA comprises at least 80% chemically modified nucleotides. In certain embodiments, the siRNA is fully chemically modified.

In certain embodiments, the siRNA comprises at least 70% 2′-O-methyl nucleotide modifications. In certain embodiments, the antisense strand comprises at least 70% 2′-O-methyl nucleotide modifications. In certain embodiments, the antisense strand comprises about 70% to 90% 2′-O-methyl nucleotide modifications. In certain embodiments, the sense strand comprises at least 65% 2′-O-methyl nucleotide modifications. In certain embodiments, the sense strand comprises 100% 2′-O-methyl nucleotide modifications.

In certain embodiments, the sense strand comprises one or more nucleotide mismatches between the antisense strand and the sense strand.

In certain embodiments, the antisense strand comprises a 5′ phosphate, a 5′-alkyl phosphonate, a 5′ alkylene phosphonate, or a 5′ alkenyl phosphonate. In certain embodiments, the antisense strand comprises a 5′ vinyl phosphonate.

In certain embodiments, the functional moiety is linked to the 5′ end and/or 3′ end of the oligonucleotide.

In certain embodiments, the functional moiety is linked to the 5′ end and/or 3′ end of the sense strand or to the 5′ end and/or 3′ end of the antisense strand.

In certain embodiments, the functional moiety is linked to the 3′ end of the sense strand.

In certain embodiments, the functional moiety is linked to the antisense strand and/or sense strand by a linker.

In certain embodiments, the linker comprises a divalent or trivalent linker.

In certain embodiments, the divalent or trivalent linker is selected from the group consisting of:

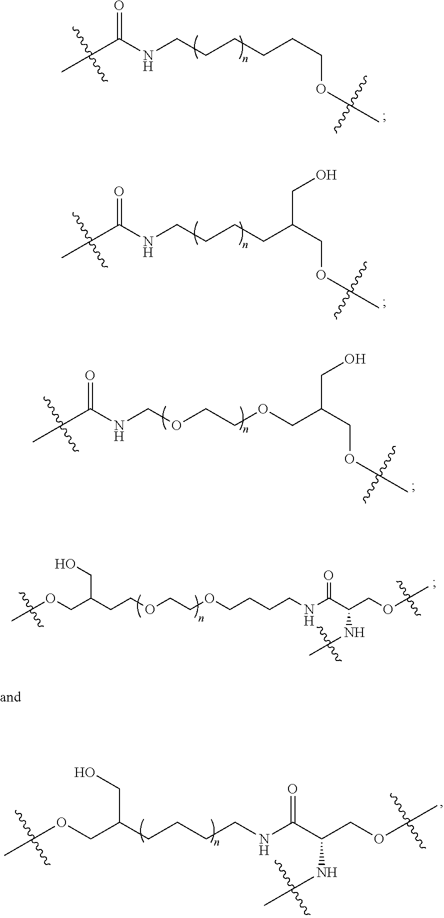

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, when the linker is a trivalent linker, the linker further links a phosphodiester or phosphodiester derivative. In certain embodiments, the phosphodiester or phosphodiester derivative is selected from the group consisting of:

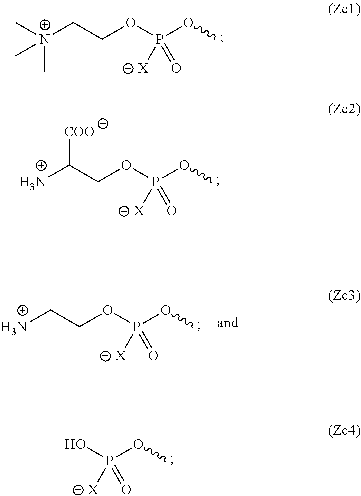

wherein X is O, S or BH₃.

In certain embodiments, the nucleotides at positions 1 and 2 from the 3′ end of sense strand, and the nucleotides at positions 1 and 2 from the 5′ end of antisense strand are connected to adjacent ribonucleotides via phosphorothioate linkages.

In certain embodiments, the two or more oligonucleotides in the branched oligonucleotide are connected to one another by one or more moieties independently selected from a linker, a spacer and a branching point. In certain embodiments, the linker comprises an ethylene glycol chain, an alkyl chain, a peptide, an RNA, a DNA, a phosphate, a phosphonate, a phosphoramidate, an ester, an amide, a triazole, or combinations thereof. In certain embodiments, the branching point comprises a polyvalent organic species or derivative thereof. In certain embodiments, the spacer comprises an ethylene glycol chain, an alkyl chain, a peptide, an RNA, a DNA, a phosphate, a phosphonate, a phosphoramidate, an ester, an amide, a triazole, or a combination thereof.

In certain embodiments, the linker comprises the structure L1:

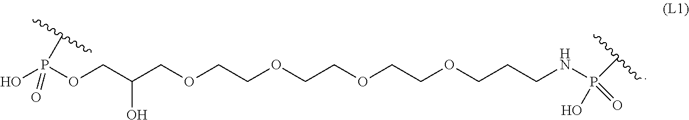 [SMILES: *P(=O)(O)NCCCOCCOCCOCCOCC(O)COP(*)(=O)O]

In certain embodiments, the linker comprises the structure L2:

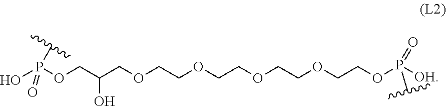 [SMILES: *P(=O)(O)OCCOCCOCCOCCOCC(O)COP(*)(=O)O]

In certain embodiments, the branched oligonucleotide consists of two oligonucleotides. In certain embodiments, the branched oligonucleotide consists of three oligonucleotides. In certain embodiments, the branched oligonucleotide consists of four oligonucleotides.

In certain embodiments, the oligonucleotides in the branched oligonucleotide are siRNA.

In certain embodiments, the oligonucleotide conjugate or branched oligonucleotide is administered by intravitreal injection.

In certain embodiments, the oligonucleotide conjugate or branched oligonucleotide is delivered to an eye cell after administration to the subject.

In certain embodiments, the eye cell is selected from the group consisting of a Müller glia cell, a rod photoreceptor cell, a cone photoreceptor cell, a ganglion cell, an amacrine cell, a bipolar cell, and a horizontal cell.

In certain embodiments, the eye cell is selected from the group consisting of a glutamine synthetase (GS)-expressing eye cell, a rhodopsin-expressing eye cell, a cone arrestin (CA)-expressing eye cell, a Vglut2-expressing eye cell, a VGAT-expressing eye cell, a protein kinase C alpha (PKCa)-expressing eye cell, and a Lim1-expressing eye cell.

In certain embodiments, the eye cell is a Müller glia cell, and: i) the oligonucleotide conjugate comprises DHA, DCA, α-tocopheryl succinate, or LA; or ii) the branched oligonucleotide consists of two, three, or four oligonucleotides. In certain embodiments, the DHA, α-tocopheryl succinate, and LA are phosphatidylcholine (PC) esterified DHA (PC-DHA), α-tocopheryl succinate (PC-TS), and LA (PC-LA).

In certain embodiments, the eye cell is a rod photoreceptor cell, and: i) the oligonucleotide conjugate comprises DCA; or ii) the branched oligonucleotide consists of three or four oligonucleotides.

In certain embodiments, the eye cell is a cone photoreceptor cell, and: i) the oligonucleotide conjugate comprises retinoic acid, DHA, DCA, α-tocopheryl succinate, or LA; or ii) the branched oligonucleotide consists of two, three or four oligonucleotides. In certain embodiments, the retinoic acid and α-tocopheryl succinate are phosphatidylcholine (PC) esterified retinoic acid (PC-RA) and α-tocopheryl succinate (PC-TS). In certain embodiments, the oligonucleotide conjugate comprises two DHA functional moieties. In certain embodiments, the eye cell is a ganglion cell, and the oligonucleotide conjugate comprises α-tocopheryl succinate. In certain embodiments, the α-tocopheryl succinate is phosphatidylcholine (PC) esterified α-tocopheryl succinate (PC-TS). In certain embodiments, the lithocholic acid (LA) is phosphatidylcholine (PC) esterified lithocholic acid (PC-LA). In certain embodiments, the natural lithocholic acid (LA) is phosphatidylcholine (PC) esterified natural lithocholic acid (PC-natural LA). In certain embodiments, the isomeric lithocholic acid (LA) is phosphatidylcholine (PC) esterified isomeric lithocholic acid (PC-isomeric LA).

In certain embodiments, the eye cell is an amacrine cell, and: i) the oligonucleotide conjugate comprises retinoic acid, DHA, DCA, α-tocopheryl succinate, or LA; or ii) the branched oligonucleotide consists of two, three or four oligonucleotides.

In certain embodiments, the retinoic acid, DHA, α-tocopheryl succinate, and LA are phosphatidylcholine (PC) esterified retinoic acid (PC-RA), DHA (PC-DHA), α-tocopheryl succinate (PC-TS), and LA (PC-LA). In certain embodiments, the oligonucleotide conjugate comprises two DHA functional moieties or two PC-DHA functional moieties.

In certain embodiments, the eye cell is a bipolar cell, and: i) the oligonucleotide conjugate comprises triple amine, retinoic acid, DHA, DCA, α-tocopheryl succinate, or LA; or ii) the branched oligonucleotide consists of two, three or four oligonucleotides. In certain embodiments, the retinoic acid, DHA, α-tocopheryl succinate, and LA are phosphatidylcholine (PC) esterified retinoic acid (PC-RA), DHA (PC-DHA), α-tocopheryl succinate (PC-TS), and LA (PC-LA). In certain embodiments, the oligonucleotide conjugate comprises two DHA functional moieties or two PC-DHA functional moieties.

In certain embodiments, the eye cell is a horizontal cell, and: i) the oligonucleotide conjugate comprises DCA; or ii) the branched oligonucleotide consists of two oligonucleotides.

In certain embodiments, the oligonucleotide conjugate comprises the structure:

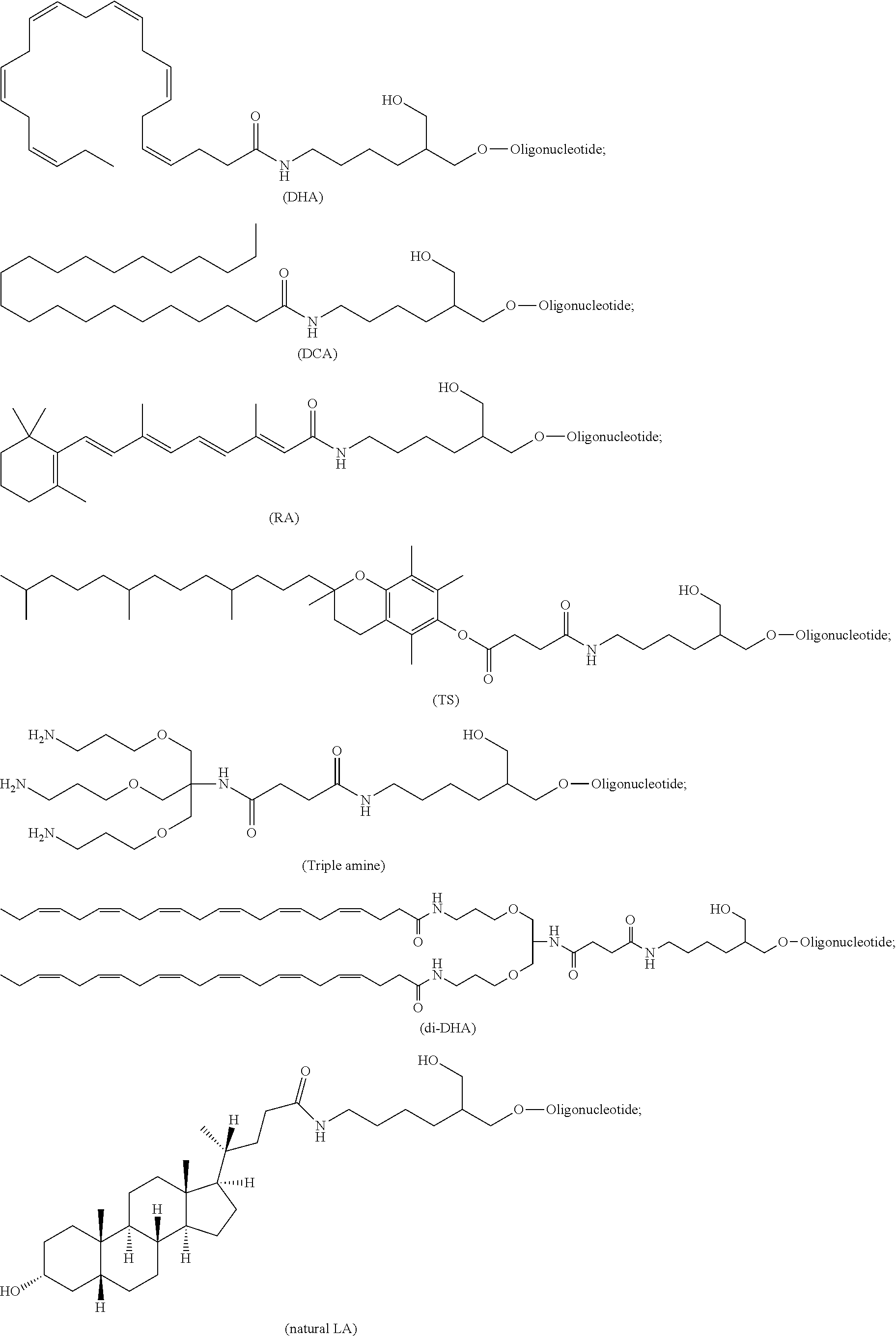

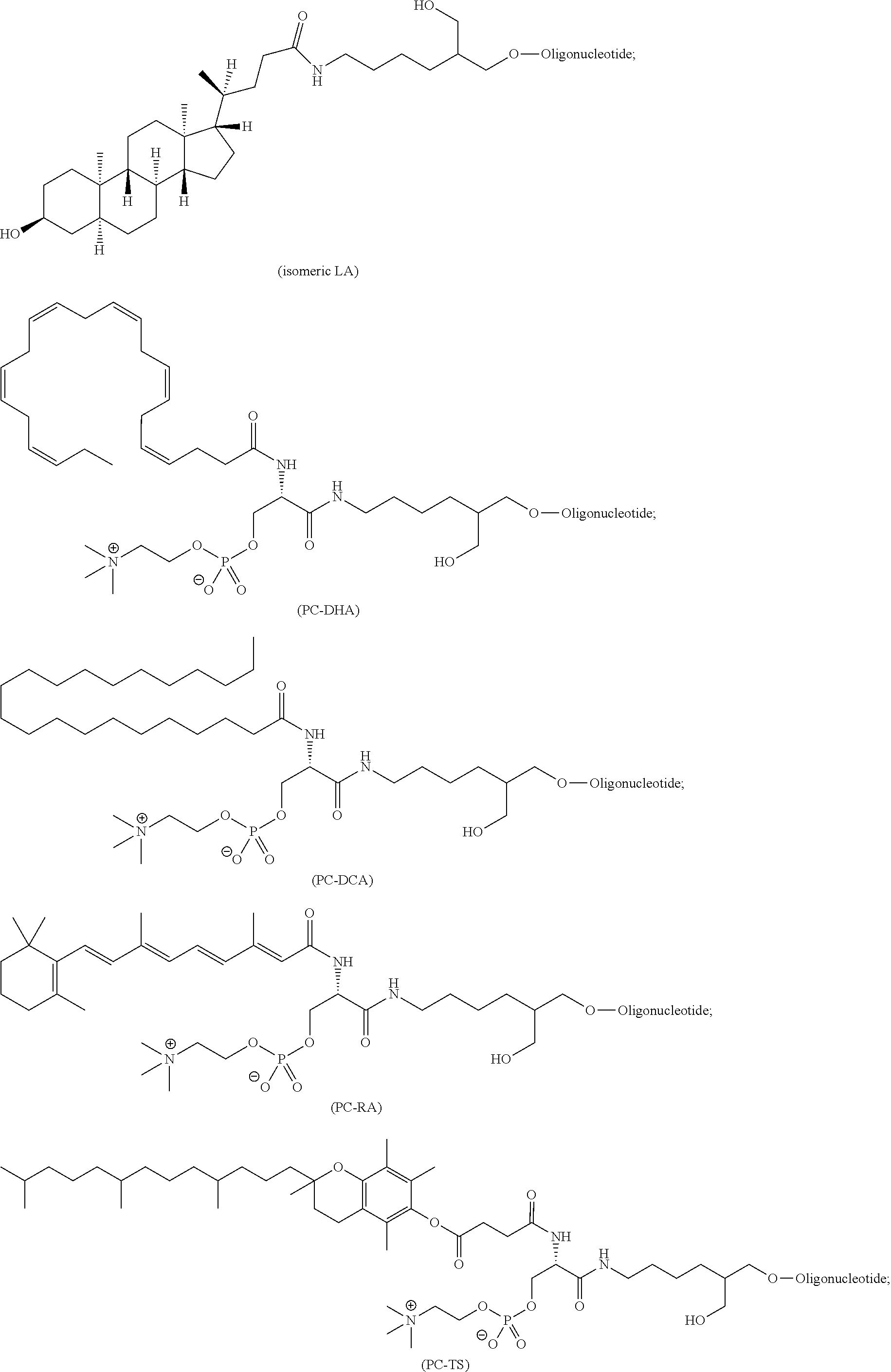

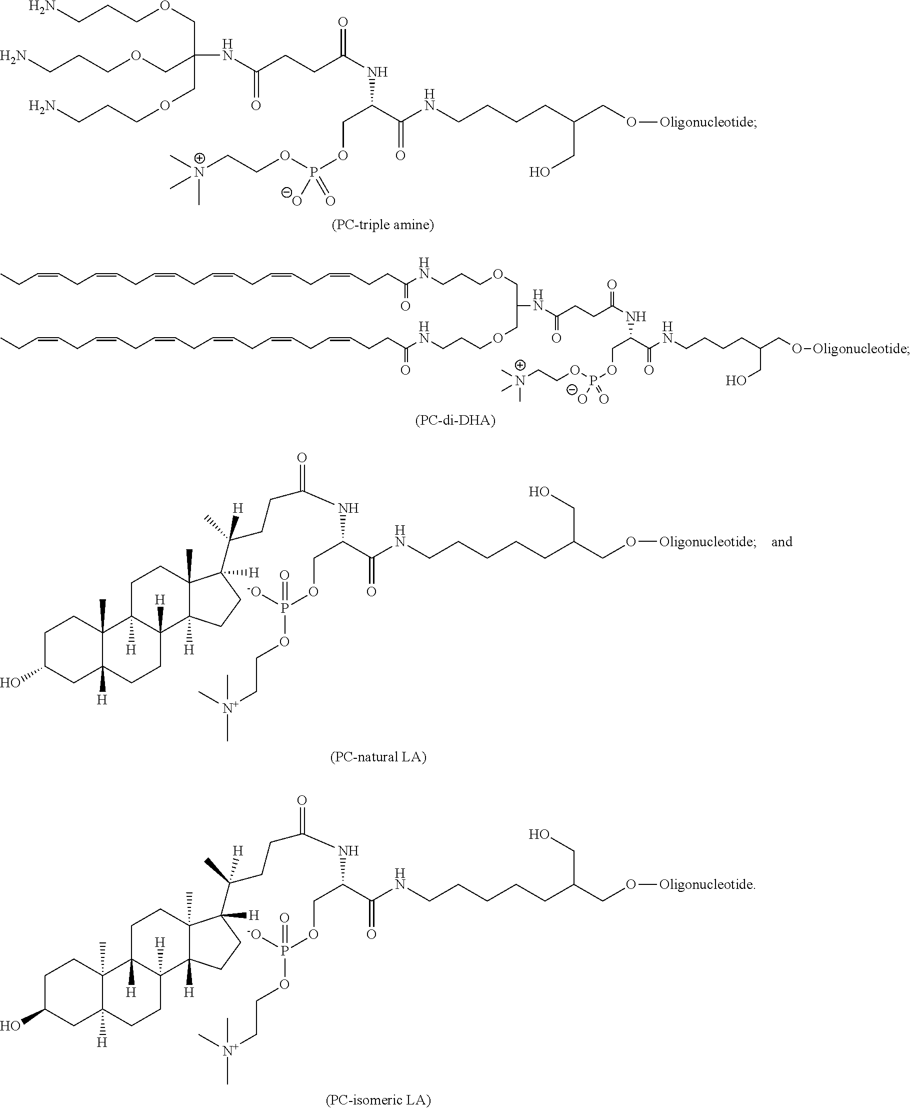

In certain embodiments, the branched oligonucleotide comprises the structure:

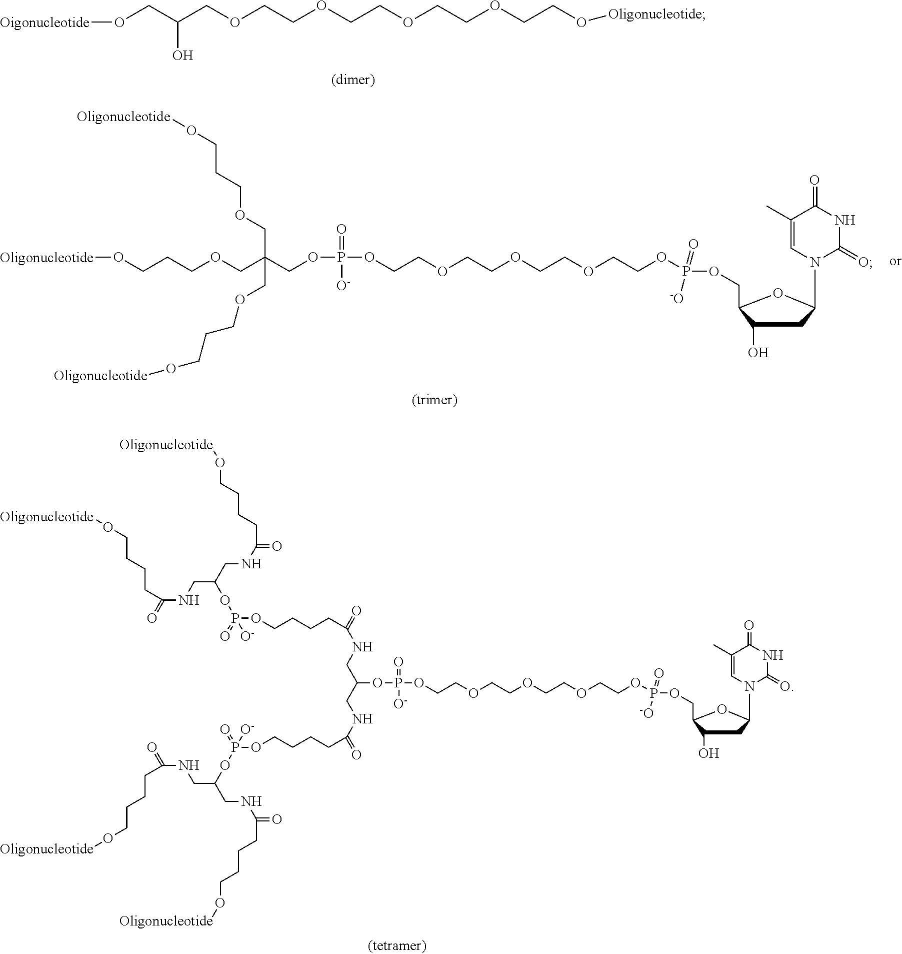

In certain embodiments, the expression of the target nucleic acid is reduced by at least 20%, at least 30%, at least 40%, or at least 50%.

In certain embodiments, the oligonucleotide conjugate has selective affinity for a retinal protein.

In certain embodiments, the subject comprises an eye disorder. In certain embodiments, administration of the oligonucleotide conjugate or the branched oligonucleotide results in the treatment of an eye disorder in the subject.

In certain embodiments, the eye disorder is selected from the group consisting of age-related macular degeneration, diabetic retinopathy, central cataract, normal-tension glaucoma, macular edema, and glaucoma.

In one aspect, the disclosure provides an oligonucleotide conjugate comprising: i) an oligonucleotide comprising a 5′ and a 3′ end and complementarity to a target nucleic acid; and ii) a di-docosahexaenoic acid (di-DHA) functional moiety linked to the oligonucleotide.

In certain embodiments, the di-DHA functional moiety is phosphatidylcholine (PC) esterified di-DHA (PC-di-DHA).

In certain embodiments, the oligonucleotide conjugate comprises the structure:

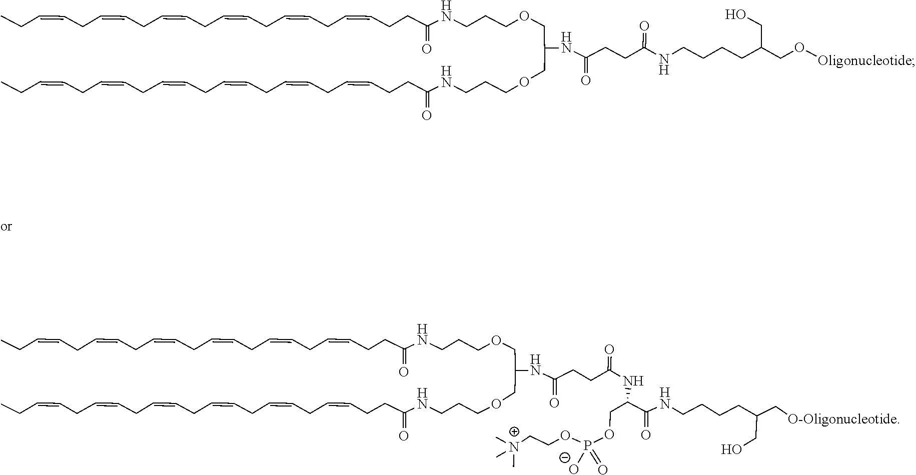

In certain embodiments, the oligonucleotide corresponds to an antisense oligonucleotide or a siRNA. In certain embodiments, the siRNA comprises a sense strand and an antisense strand. In certain embodiments, the functional moiety is linked to the 5′ end and/or 3′ end of the sense strand or to the 5′ end and/or 3′ end of the antisense strand. In certain embodiments, the functional moiety is linked to the 3′ end of the sense strand.

In one aspect, the disclosure provides an oligonucleotide conjugate comprising: i) an oligonucleotide comprising a 5′ and a 3′ end and complementarity to a target nucleic acid; and ii) a triple amine functional moiety linked to the oligonucleotide.

In certain embodiments, the triple amine functional moiety is phosphatidylcholine (PC) esterified triple amine (PC-triple amine).

In certain embodiments, the oligonucleotide conjugate comprises the structure:

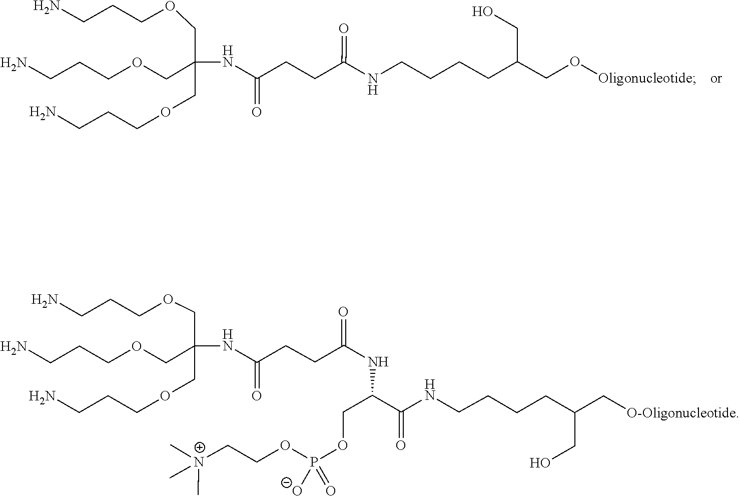

In certain embodiments, the oligonucleotide corresponds to an antisense oligonucleotide or a siRNA. In certain embodiments, the siRNA comprises a sense strand and an antisense strand. In certain embodiments, the functional moiety is linked to the 5′ end and/or 3′ end of the sense strand or to the 5′ end and/or 3′ end of the antisense strand.

In certain embodiments, the functional moiety is linked to the 3′ end of the sense strand.

### BRIEF DESCRIPTION OF THE DRAWINGS

FIG. 1 shows retinal cross sections with 12 different siRNA distributions 3 days after injection of 0.3 nanomoles of siRNA (left panels: retinoic acid (RA), Docosahexaenoic acid (DHA), phosphocholine (PC); α-tocopheryl succinate (TS); docosanoic acid (DCA)). To the right: One example per group showing entire retinal cross section with distribution across the entire retina. All siRNAs are labeled with Cy3 and are shown in red. Glutamine synthetase (GS) expression, is shown in green. Nuclear DAPI is shown in blue. To the right: One example per group showing entire retinal cross section with distribution across the entire retina.

FIG. 2 shows the enrichment of siRNA in different retinal cell types arranged by cell type. The bars show relative protein levels of cell type specific markers calibrated to total retinal extracts of an un-injected mouse retina.

FIG. 3 shows the enrichment of siRNA in different retinal cell types arranged by modifications. The bars show relative protein levels of cell type specific markers calibrated to total retinal extracts of an un-injected mouse retina. For each modification (i.e., monomer, dimer, trimer, etc.) each bar, from left to right, represents rhodopsin (rods), cone arrestin (CA)(cones), glutamine synthetase (GS) (muller cells), Vglut2 (ganglion cells), VGAT (amacrine cells), protein kinase C alpha (PKCa) (bipolar cells), and Lim1 (horizontal cells).

FIG. 4 shows examples of siRNA distributions in retinal cross sections 3 days after injection of 0.3 nanomole of siRNA. siRNA is labeled shows with Cy3 and is shown in red. All siRNAs are targeting the Huntington gene. Shown is the non-targeting control (NTC) with the PC-TS modification, the trimer, and the tetramer. Cone segments are highlighted in green labeled by PNA (peanut agglutinin lectin), Müller glia cells are shown in cyan, labeled by Glutamine synthetase (GS) and nuclei are shown in blue labeled by DAPI.

FIG. 5 shows examples of siRNA distributions in retinal cross sections 3 days after injection of 0.3 nanomole of siRNA without GS staining in cyan. Additionally, for the trimer and tetramer only the higher magnification of the outer nuclear layer (ONL) is shown to highlight the distribution of the siRNA in the photoreceptor layer. On half of the panel only the siRNA is shown to better visualize signal.

FIG. 6 shows antibody staining on retinal cross sections for HTT protein two weeks after injection with the Htt-siRNA. First column shows staining in control mice injected with the NTC-siRNA. Second column, expression of HTT protein after knockdown with PC-RA-Htt siRNA. Shown are examples of 2 different mice each injected with ˜0.3 nanomoles of the Htt-siRNA.

FIG. 7 shows the quantification by western blotting of total HTT protein two weeks after injection with the Htt-siRNA. Same experimental setting as in FIG. 6 (different mice of the same injected batch) quantifying total HTT protein remaining from total retinal extracts.

FIG. 8 shows the quantification by bDNA assay to quantify total Htt mRNA levels two weeks after injection with the Htt-siRNA using 0.1 nanomole per injection of stated siRNA modification.

FIG. 9 shows quantification by bDNA assay to quantify total Htt mRNA levels 3 days after injection with the Htt-siRNA using 0.3 nanomoles per injection of stated siRNA modification. Each dot represents 1 retina.

FIG. 10 shows the quantification by bDNA assay to quantify total Htt mRNA levels 100 days after injection with the Htt-siRNA using 0.3 nanomoles per injection of stated siRNA modification. Each dot represents 1 retina.

FIG. 11 shows representative fundus images over time of eyes injected with the Cy3 labeled siRNAs with modifications as indicated. Exposure of fluorescence signal is the same for all 4 siRNA at any given time point, but not over time. This figure complements FIG. 10 showing the fundus images of the mice used in FIG. 10. All mice were injected with 0.3 nanomoles of siRNA intravitreally.

FIG. 12 shows dose escalation study of for HTT-knockdown in retina. Mice were injected with amounts indicated in FIG. 1-60 microgram [note: not nanomoles] of Cy3 labeled Tetramer with Htt-siRNA) in a total volume of 2 microliter. Five mice were injected per amount of siRNA. Tissue was harvested at 2 weeks post-injection to perform quantification by western blotting of remaining HTT protein in retina. Injections with 15-30 microgram correspond roughly to the same knockdown seen with ˜0.3 nanomoles in previous experiments.

FIG. 13 shows fundus images of dose escalation study shown in FIG. 12. Images were taken before euthanasia at 2 weeks post-injection. Regular brightfield fundus image as well as Cy3 image is shown for each concentration.

FIG. 14 shows retinal cross sections of eyes from of dose escalation study shown in FIGS. 12 and 13. Images show Cy3 distribution across entire retinal section, indicating that the siRNA is taken up uniformly across the entire eye.

FIG. 15 shows retinal cross sections of eyes from of dose escalation study shown in FIG. 14 stained with Iba1 (green) to identify Iba1 positive cells that migrate to the outer nuclear layer (ONL) where photoreceptors reside. Half of each panel (dotted line) shows only the Iba1 signal to better visualize the signal Blue shows nuclear DAPI.

FIG. 16 shows retinal cross sections of eyes from of dose escalation study shown in FIG. 14 stained with GFAP (red) to identify reactive gliosis in Müller glia cells. siRNA is not shown as these are sections from the same eyes as shown in FIG. 15. Blue shows nuclear DAPI and green marks cone photoreceptor segment with peanut agglutinin lectin (PNA).

FIG. 17 shows measurements of photoreceptor and retinal function by electroretinography under scotopic (0.01 cd·s/m2-1 cd·s/m2) and photopic conditions (3 & 10 flashes). A-waves and b-waves are recorded at several amounts injected.

FIG. 18 shows fluorescence intensity of Tetramer-Htt-Cy3 after intravitreal delivery in pig eye. Delivery of amount of siRNA is shown on top of each panel (100-1500 microgram of Tetramer. Top row shows the Cy3 fluorescence of unfixed tissue right after opening the eye. On the bottom of the figure is a higher magnification of a region from the top panel.

FIG. 19 shows the knockdown of Huntington protein in Swine as measured by western blot analysis from eyes shown in FIG. 18. Knockdown was compared to Huntington protein levels in the NTC that was injected with 250 ug of the Tetramer-siRNA-Cy3. Top figure shows knockdown in bar graphs seen in the four major retinal quadrants (DT: Dorsal-Temporal; DN: Dorsal-Nasal; VT: Temporal-Nasal; VN: Ventral-Nasal). Middle panel shows knockdown on a flat mount cartoon with corresponding values of the regional knockdown shown in the bar graph. Bottom panel: Average knockdown of Huntington protein across the entire retina calculated by averaging the knockdown seen in each quadrant per retina. Data shown represents one biological sample for each amount of siRNA delivered. Error bar in first panel is generated by technical replicates. Error bar in last panel is generated by averaging the 4 data points for each quadrant per retina.

FIG. 20 shows antibody staining for Huntington protein on section of eyes injected with different amount as shown in FIG. 19. Area of section is shown in middle panel of FIG. 19.

FIG. 21 shows antibody staining for GFAP (glial fibrillary acidic protein) and Iba1 (ionized calcium binding adaptor protein 1) (as shown in mouse on FIGS. 15 and 16) expression on retinal section of eyes injected with different amount as shown in FIGS. 18 and 19 to determine dose dependent toxicity. GFAP and Iba1 are both shown in green as indicated to the left of each row. Red staining shown siRNA distribution across the retinal section. Nuclei are marked with nuclear DAPI. Half of each panel shows only the signal of interest (siRNA, GFAP or Iba1) to better visualize the signal.

FIG. 22 shows initial knockdown efficiency in vitro of siRNA duplexes formed from the sense and antisense strands shown in Table 3 and 4.

FIG. 23 shows dose response curves for duplexes 2, 3, 9, and 10 from FIG. 22.

FIG. 24 shows an RNA-Scope in situ hybridization on retinal cross-sections of mice to detect the siRNA-tetramer against S6K1. Top row shows sections from 3 mice injected with the NTC for S6K1 in the tetramer configuration. Middle row shows sections from 3 mice injected with the 3 μg/eye with the siRNA against S6K1 in the tetramer configuration. Last row shows sections from 3 mice injected with the 6 μg/eye with the siRNA against S6K1 in the tetramer configuration. The siRNA was delivered intravitreally and animals were euthanized 2 weeks post injection.

FIG. 25A-FIG. 25B show knockdown of S6K1 in mouse after intravitreal injection of 6 μg of siRNA in the tetramer configuration. FIG. 25A shows S6K1 protein levels as detected by western blot 2-weeks post injection. FIG. 25B shows similar data as first graph at 2 months post-injection. Each dot in the graphs represent one biological sample (retina) from one animal.

FIG. 26 shows the knockdown of S6K1 protein in non-human primate (NHP). Western blot data with retinal protein extracts form the superior-temporal (ST) regions (AKA: dorsal-temporal) of one NHP injected intravitreally with 225 ug of S6K1-tetramer (in 75 μL) and 6 naïve NHP retinas from the same region. First set of bar graphs shows a comparison between the uninjected contralateral eye and the S6K1 siRNA injected one to allow for a direct intra-animal comparison between both eyes. The second bar graph shows a comparison between the 6 naïve NHPs and the S6K1 siRNA injected one. NHP eyes were harvested 1-month post-injection. Shown is also the phosphorylation of ribosomal protein S6, which is a canonical target of S6K1. Similar to the S6K1 knockdown data, intra-animal comparison is shown to the left and comparison with several NHPs is shown to the right.

FIG. 27 shows the knockdown of S6K1 protein on retinal cross section of non-human primate (NHP) after siRNA treatment. Data is generated with the one injected eye and the uninjected contralateral eye. Sections were obtained from the central regions as shown for the pig in FIG. 19. To the left: entire cross section encompassing the fovea. To the right: higher magnification of temporal and nasal regions as well as the fovea. Top row shows uninjected eye and bottom row eye injected intravitreally with 225 μg of S6K1-tetramer (in 75 μL).

FIG. 28 shows the reduction in phosphorylated S6 protein (pS6) on retinal cross section of non-human primate (NHP) after siRNA treatment. Data is same as shown in FIG. 27, with the exception that the staining probes for the expression of pS6 (red signal). In each panel green and blue signal have been removed from half the panel (dotted line) to better visualize the knockdown of pS6. Blue shows nuclear DAPI and green shows cones segments marked with peanut agglutinin lectin (PNA).

FIG. 29 shows the expression of inflammatory markers in NHP after siRNA treatment with S6K1 siRNA (75 microliter, 225 μg of siRNA in tetramer configuration). Data is same as shown in FIG. 27 and FIG. 28, with the exception that the staining probes for the expression of Iba1 (red signal, first set) and GFAP (red signal, second set). Untreated contralateral eye is in first row of each set and the treated one in the second row. In each panel green and blue signal have been removed from half the panel (dotted line) to better visualize the Iba1 and GFAP signal. Blue shows nuclear DAPI and green shows cones segments marked with peanut agglutinin lectin (PNA).

### DETAILED DESCRIPTION

The present disclosure relates to oligonucleotide conjugates and branched oligonucleotides that are capable of efficient gene knockdown in the eye. Several different functional moieties and branched oligonucleotides demonstrated eye cell specific delivery upon administration.

The oligonucleotide conjugates and branched oligonucleotides described herein promote simple, efficient, non-toxic delivery of oligonucleotides (e.g., siRNA), and promote potent silencing of therapeutic targets in a range of eye cell types in vivo.

Unless otherwise specified, nomenclature used in connection with cell and tissue culture, molecular biology, immunology, microbiology, genetics and protein and nucleic acid chemistry and hybridization described herein are those well-known and commonly used in the art. Unless otherwise specified, the methods and techniques provided herein are performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, delivery, and treatment of patients.

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the disclosure may be more readily understood, certain terms are first defined.

As used herein in the context of oligonucleotide sequences, “A” represents a nucleoside comprising the base adenine (e.g., adenosine or a chemically-modified derivative thereof), “G” represents a nucleoside comprising the base guanine (e.g., guanosine or a chemically-modified derivative thereof), “U” represents a nucleoside comprising the base uracil (e.g., uridine or a chemically-modified derivative thereof), and “C” represents a nucleoside comprising the base adenine (e.g., cytidine or a chemically-modified derivative thereof).

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. The siRNA is a duplex formed by a sense strand and antisense strand which have sufficient complementarity to each other to form said duplex. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” or “chemically modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide, which may be derivatized include: the 5 position, e.g., 5-(2-amino)propyl uridine, 5-bromo uridine, 5-propyne uridine, 5-propenyl uridine, etc.; the 6 position, e.g., 6-(2-amino)propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs, such as those described in Herdewijn, Antisense Nucleic Acid Drug Dev., 2000 Aug. 10(4):297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, or COOR, wherein R is substituted or unsubstituted C₁-C₆ alkyl, alkenyl, alkynyl, aryl, etc. Other modifications include those described in U.S. Pat. Nos. 5,858,988, and 6,291,438. In certain embodiments, the nucleotide analog comprises a 2′-O-methyl modification. In certain embodiments, the nucleotide analog comprises a 2′-fluoro modification.

The phosphate group of the nucleotide may also be modified, e.g., by substituting one or more of the oxygens of the phosphate group with sulfur (e.g., phosphorothioate), or by making other substitutions, which allow the nucleotide to perform its intended function, such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs In vivo or in vitro.

The term “oligonucleotide” refers to a short polymer of nucleotides and/or nucleotide analogs. The term “oligonucleotide” includes, but is not limited to, antisense oligonucleotide (ASO), siRNA, and micro-RNA.

The term “RNA analog” refers to a polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoroamidate, and/or phosphorothioate linkages. Some RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An RNAi agent, e.g., an RNA silencing agent, having a strand, which is “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g., RNA interference (RNAi), transcriptional gene silencing (TGS), post-transcriptional gene silencing (PTGS), quelling, co-suppression, and translational repression) mediated by RNA molecules, which result in the inhibition or “silencing” of the expression of a corresponding protein-coding gene. RNA silencing has been observed in many types of organisms, including plants, animals, and fungi.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, a “target” refers to a particular nucleic acid sequence (e.g., a gene, an mRNA, a miRNA or the like) that an oligonucleotide conjugate or branched oligonucleotide of the disclosure binds to and/or otherwise effects the expression of In certain embodiments, the target is expressed in the eye. In certain embodiments, target is expressed in a specific eye cell. In other embodiments, a target is associated with a particular disease or disorder in a subject.

As used herein, the term “target gene” is a gene whose expression is to be substantially inhibited or “silenced.” This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g., mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g., an orthologue or paralogue) of the target gene.

As used herein, the term “RNA silencing agent” refers to an RNA, which is capable of inhibiting or “silencing” the expression of a target gene. In certain embodiments, the RNA silencing agent is capable of preventing complete processing (e.g., the full translation and/or expression) of a mRNA molecule through a post-transcriptional silencing mechanism. RNA silencing agents include small (<50 b.p.), noncoding RNA molecules, for example RNA duplexes comprising paired strands, as well as precursor RNAs from which such small non-coding RNAs can be generated. Exemplary RNA silencing agents include siRNAs, miRNAs, siRNA-like duplexes, antisense oligonucleotides, GAPMER molecules, and dual-function oligonucleotides, as well as precursors thereof. In one embodiment, the RNA silencing agent is capable of inducing RNA interference. In another embodiment, the RNA silencing agent is capable of mediating translational repression.

As used herein, the term “rare nucleotide” refers to a naturally occurring nucleotide that occurs infrequently, including naturally occurring deoxyribonucleotides or ribonucleotides that occur infrequently, e.g., a naturally occurring ribonucleotide that is not guanosine, adenosine, cytosine, or uridine. Examples of rare nucleotides include, but are not limited to, inosine, 1-methyl inosine, pseudouridine, 5,6-dihydrouridine, ribothymidine, 2N-methylguanosine and 2,2N,N-dimethylguanosine.

The term “engineered,” as in an engineered RNA precursor, or an engineered nucleic acid molecule, indicates that the precursor or molecule is not found in nature, in that all or a portion of the nucleic acid sequence of the precursor or molecule is created or selected by a human. Once created or selected, the sequence can be replicated, translated, transcribed, or otherwise processed by mechanisms within a cell. Thus, an RNA precursor produced within a cell from a transgene that includes an engineered nucleic acid molecule is an engineered RNA precursor.

As used herein, the term “microRNA” (“miRNA”), also known in the art as “small temporal RNAs” (“stRNAs”), refers to a small (10-50 nucleotide) RNA, which are genetically encoded (e.g., by viral, mammalian, or plant genomes) and are capable of directing or mediating RNA silencing. An “miRNA disorder” shall refer to a disease or disorder characterized by an aberrant expression or activity of a miRNA.

As used herein, the term “dual functional oligonucleotide” refers to an RNA silencing agent having the formula T-L-μ, wherein T is an mRNA targeting moiety, L is a linking moiety, and μ is a miRNA recruiting moiety. As used herein, the terms “mRNA targeting moiety,” “targeting moiety,” “mRNA targeting portion” or “targeting portion” refer to a domain, portion or region of the dual functional oligonucleotide having sufficient size and sufficient complementarity to a portion or region of an mRNA chosen or targeted for silencing (i.e., the moiety has a sequence sufficient to capture the target mRNA).

As used herein, the term “linking moiety” or “linking portion” refers to a domain, portion or region of the RNA-silencing agent which covalently joins or links the mRNA.

As used herein, the term “antisense strand” of an RNA silencing agent, e.g., an siRNA, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of an RNA silencing agent, e.g., an siRNA or RNA silencing agent, refers to a strand that is complementary to the antisense strand or first strand. Antisense and sense strands can also be referred to as first or second strands, the first or second strand having complementarity to the target sequence and the respective second or first strand having complementarity to said first or second strand. miRNA duplex intermediates or siRNA-like duplexes include a miRNA strand having sufficient complementarity to a section of about 10-50 nucleotides of the mRNA of the gene targeted for silencing and a miRNA* strand having sufficient complementarity to form a duplex with the miRNA strand.

As used herein, the term “guide strand” refers to a strand of an RNA silencing agent, e.g., an antisense strand of an siRNA duplex or siRNA sequence, that enters into the RISC complex and directs cleavage of the target mRNA.

As used herein, the term “asymmetry,” as in the asymmetry of the duplex region of an RNA silencing agent (e.g., the stem of an shRNA), refers to an inequality of bond strength or base pairing strength between the termini of the RNA silencing agent (e.g., between terminal nucleotides on a first strand or stem portion and terminal nucleotides on an opposing second strand or stem portion), such that the 5′ end of one strand of the duplex is more frequently in a transient unpaired, e.g., single-stranded, state than the 5′ end of the complementary strand. This structural difference determines that one strand of the duplex is preferentially incorporated into a RISC complex. The strand whose 5′ end is less tightly paired to the complementary strand will preferentially be incorporated into RISC and mediate RNAi.

As used herein, the term “bond strength” or “base pair strength” refers to the strength of the interaction between pairs of nucleotides (or nucleotide analogs) on opposing strands of an oligonucleotide duplex (e.g., an siRNA duplex), due primarily to H-bonding, van der Waals interactions, and the like, between said nucleotides (or nucleotide analogs).

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein the term “destabilizing nucleotide” refers to a first nucleotide or nucleotide analog capable of forming a base pair with second nucleotide or nucleotide analog such that the base pair is of lower bond strength than a conventional base pair (i.e., Watson-Crick base pair). In certain embodiments, the destabilizing nucleotide is capable of forming a mismatch base pair with the second nucleotide. In other embodiments, the destabilizing nucleotide is capable of forming a wobble base pair with the second nucleotide. In yet other embodiments, the destabilizing nucleotide is capable of forming an ambiguous base pair with the second nucleotide.

As used herein, the term “base pair” refers to the interaction between pairs of nucleotides (or nucleotide analogs) on opposing strands of an oligonucleotide duplex (e.g., a duplex formed by a strand of a RNA silencing agent and a target mRNA sequence), due primarily to H-bonding, van der Waals interactions, and the like between said nucleotides (or nucleotide analogs). As used herein, the term “bond strength” or “base pair strength” refers to the strength of the base pair.

As used herein, the term “mismatched base pair” refers to a base pair consisting of non-complementary or non-Watson-Crick base pairs, for example, not normal complementary G:C, A:T or A:U base pairs. As used herein the term “ambiguous base pair” (also known as a non-discriminatory base pair) refers to a base pair formed by a universal nucleotide.

As used herein, term “universal nucleotide” (also known as a “neutral nucleotide”) include those nucleotides (e.g., certain destabilizing nucleotides) having a base (a “universal base” or “neutral base”) that does not significantly discriminate between bases on a complementary polynucleotide when forming a base pair. Universal nucleotides are predominantly hydrophobic molecules that can pack efficiently into antiparallel duplex nucleic acids (e.g., double-stranded DNA or RNA) due to stacking interactions. The base portion of universal nucleotides typically comprise a nitrogen-containing aromatic heterocyclic moiety.

As used herein, the terms “sufficient complementarity” or “sufficient degree of complementarity” mean that the RNA silencing agent has a sequence (e.g., in the antisense strand, mRNA targeting moiety or miRNA recruiting moiety), which is sufficient to bind the desired target RNA, respectively, and to trigger the RNA silencing of the target mRNA.

As used herein, the term “translational repression” refers to a selective inhibition of mRNA translation. Natural translational repression proceeds via miRNAs cleaved from shRNA precursors. Both RNAi and translational repression are mediated by RISC. Both RNAi and translational repression occur naturally or can be initiated by the hand of man, for example, to silence the expression of target genes.

Various methodologies of the instant disclosure include a step that involves comparing a value, level, feature, characteristic, property, etc. to a “suitable control,” referred to interchangeably herein as an “appropriate control.” A “suitable control” or “appropriate control” is any control or standard familiar to one of ordinary skill in the art useful for comparison purposes. In one embodiment, a “suitable control” or “appropriate control” is a value, level, feature, characteristic, property, etc. determined prior to performing an RNAi methodology, as described herein. For example, a transcription rate, mRNA level, translation rate, protein level, biological activity, cellular characteristic or property, genotype, phenotype, etc. can be determined prior to introducing an RNA silencing agent of the disclosure into a cell or organism. In another embodiment, a “suitable control” or “appropriate control” is a value, level, feature, characteristic, property, etc. determined in a cell or organism, e.g., a control or normal cell or organism, exhibiting, for example, normal traits. In yet another embodiment, a “suitable control” or “appropriate control” is a predefined value, level, feature, characteristic, property, etc.

### Oligonucleotide Conjugates

The oligonucleotide conjugates described here comprise an oligonucleotide linked to a functional moiety. The functional moieties provide enhanced eye delivery of the oligonucleotide, including eye cell-specific delivery.

In one aspect, the disclosure provides a method for delivering an oligonucleotide conjugate to an eye of a subject, the method comprising administering the oligonucleotide conjugate to the subject, wherein the oligonucleotide conjugate comprises: i) an oligonucleotide comprising a 5′ and a 3′ end and complementarity to a target nucleic acid (e.g., target gene or target mRNA); and ii) a functional moiety linked to the oligonucleotide, wherein the functional moiety comprises any one of triple amine, retinoic acid (RA), docosahexaenoic acid (DHA), docosanoic acid (DCA), α-tocopheryl succinate (TS), or lithocholic acid (LA).

Each of the functional moieties described above are depicted below structurally. The functional moieties can have different isomeric configurations than the ones presented in this disclosure.

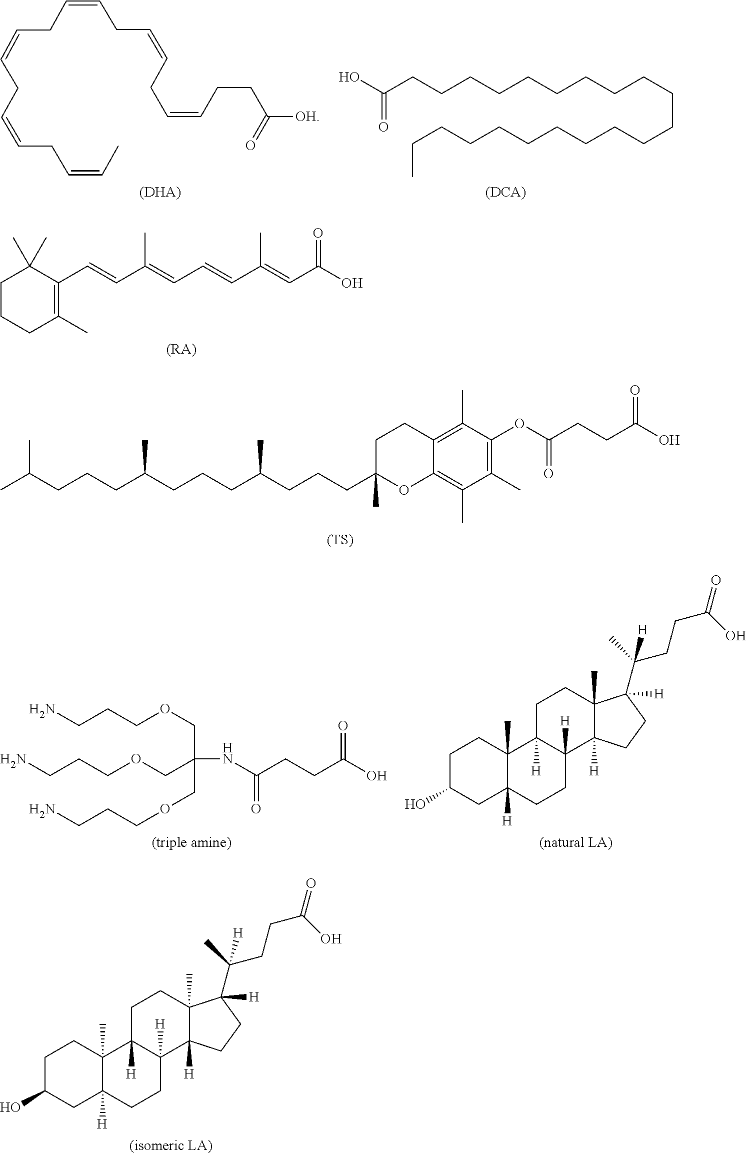

In certain embodiments, two DHA functional moieties are linked to the oligonucleotide.

In certain embodiments, the oligonucleotide comprises an antisense oligonucleotide or an siRNA.

In certain embodiments, the siRNA comprises a sense strand and an antisense strand. In certain embodiments, the antisense strand comprises about 15 nucleotides to 25 nucleotides in length (e.g., 15, 16, 17, 18, 19, 20, 21, 22, 23, 24, or 25 nucleotides in length). In certain embodiments, the antisense strand is 20 nucleotides in length, 21 nucleotides in length, or 22 nucleotides in length. In certain embodiments, the sense strand comprises about 15 nucleotides to 25 nucleotides in length (e.g., 15, 16, 17, 18, 19, 20, 21, 22, 23, 24, or 25 nucleotides in length). In certain embodiments, the sense strand is 15 nucleotides in length, 16 nucleotides in length, 18 nucleotides in length, or 20 nucleotides in length.

In certain embodiments, the siRNA comprises a double-stranded region of 15 base pairs to 20 base pairs (e.g., 15, 16, 17, 18, 19, or 20 base pairs). In certain embodiments, the siRNA comprises a double-stranded region of 15 base pairs, 16 base pairs, 18 base pairs, or 20 base pairs.

In certain embodiments, the siRNA comprises at least one blunt-end. In certain embodiments, the siRNA comprises two blunt-ends.

In certain embodiments, the siRNA comprises at least one single stranded nucleotide overhang (also referred to herein as a “single-stranded tail”). In certain embodiments, the siRNA comprises two single stranded nucleotide overhangs. In certain embodiments, the siRNA comprises about a 2-nucleotide to 5-nucleotide single stranded nucleotide overhang (e.g., a 2-, 3-, 4-, or 5-nucleotide overhang). In certain embodiments, the siRNA comprises a 2-nucleotide single stranded nucleotide overhang or a 5-nucleotide single stranded nucleotide overhang.

In certain embodiments, the siRNA comprises naturally occurring nucleotides (i.e., unmodified ribonucleotides).

In certain embodiments, the siRNA comprises at least one modified nucleotide. In certain embodiments, the modified nucleotide comprises a 2′-O-methyl modified nucleotide, a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, a morpholino nucleotide, a phosphoramidate, a non-natural base comprising nucleotide, or a mixture thereof.

In certain embodiments, the siRNA comprises at least one modified internucleotide linkage. In certain embodiments, the modified internucleotide linkage comprises a phosphorothioate internucleotide linkage. In certain embodiments, the siRNA comprises 4-16 phosphorothioate internucleotide linkages. In certain embodiments, the siRNA comprises 8-13 phosphorothioate internucleotide linkages.

In certain embodiments, the siRNA comprises at least 80% chemically modified nucleotides (e.g., 80%, 81%, 82%, 83%, 84%, 85%, 86%, 87%, 88%, 89%, 90%, 91%, 92%, 93%, 94%, 95%, 96%, 97%, 98%, 99%, or 100% chemically modified nucleotides). In certain embodiments, the siRNA is fully chemically modified.

In certain embodiments, the siRNA comprises at least 70% 2′-O-methyl nucleotide modifications (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the antisense strand comprises at least 70% 2′-O-methyl nucleotide modifications (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-O-methyl nucleotide modifications). In certain embodiments, the antisense strand comprises about 70% to 90% 2′-O-methyl nucleotide modifications. In certain embodiments, the sense strand comprises at least 65% 2′-O-methyl nucleotide modifications (e.g., 65%, 66%, 67%, 68%, 69% 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% % 2′-o-methyl nucleotide modifications). In certain embodiments, the sense strand comprises 100% 2′-O-methyl nucleotide modifications.

In certain embodiments, the sense strand comprises one or more nucleotide mismatches between the antisense strand and the sense strand.

In certain embodiments, the antisense strand comprises a 5′ phosphate, a 5′-alkyl phosphonate, a 5′ alkylene phosphonate, or a 5′ alkenyl phosphonate. In certain embodiments, the antisense strand comprises a 5′ vinyl phosphonate.

In certain embodiments, the functional moiety is linked to the 5′ end and/or 3′ end of the oligonucleotide. In certain embodiments, the functional moiety is linked to the 5′ end and/or 3′ end of the sense strand or to the 5′ end and/or 3′ end of the antisense strand. In certain embodiments, the functional moiety is linked to the 3′ end of the sense strand.

In certain embodiments, the functional moiety is linked to the antisense strand and/or sense strand by a linker.

In certain embodiments, the linker comprises a divalent or trivalent linker.

In certain embodiments, the divalent or trivalent linker is selected from the group consisting of:

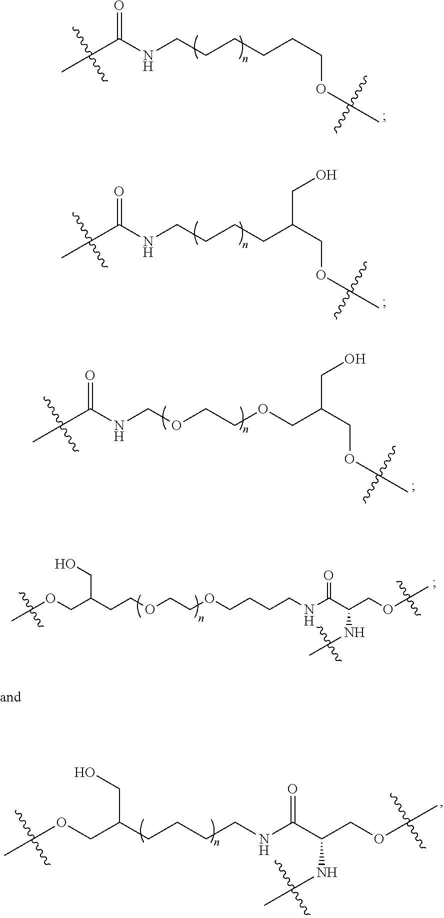

wherein n is 1, 2, 3, 4, or 5.

In certain embodiments, the linker comprises an ethylene glycol chain, an alkyl chain, a peptide, an RNA, a DNA, a phosphodiester, a phosphorothioate, a phosphoramidate, an amide, a carbamate, or a combination thereof.

In certain embodiments, when the linker is a trivalent linker, the linker further links a phosphodiester or phosphodiester derivative.

In certain embodiments, the phosphodiester or phosphodiester derivative is selected from the group consisting of:

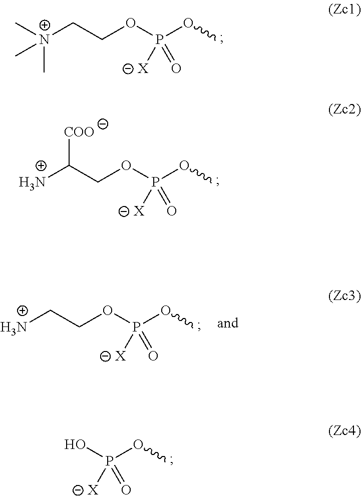

wherein X is O, S or BH₃.

The above recited moiety Zc1 is phosphatidylcholine (PC). Any one of the functional moieties described herein may comprise a phosphatidylcholine (PC) esterified derivative, i.e., phosphatidylcholine (PC) esterified triple amine (PC-triple amine), phosphatidylcholine (PC) esterified retinoic acid (PC-RA), phosphatidylcholine (PC) esterified docosahexaenoic acid (PC-DHA), phosphatidylcholine (PC) esterified docosanoic acid (PC-DCA), phosphatidylcholine (PC) esterified α-tocopheryl succinate (PC-TS), phosphatidylcholine (PC) esterified lithocholic acid (PC-TS).

In certain embodiments, the nucleotides at positions 1 and 2 from the 3′ end of sense strand, and the nucleotides at positions 1 and 2 from the 5′ end of antisense strand are connected to adjacent ribonucleotides via phosphorothioate linkages.

In certain embodiments, the oligonucleotide conjugate comprises the structure:

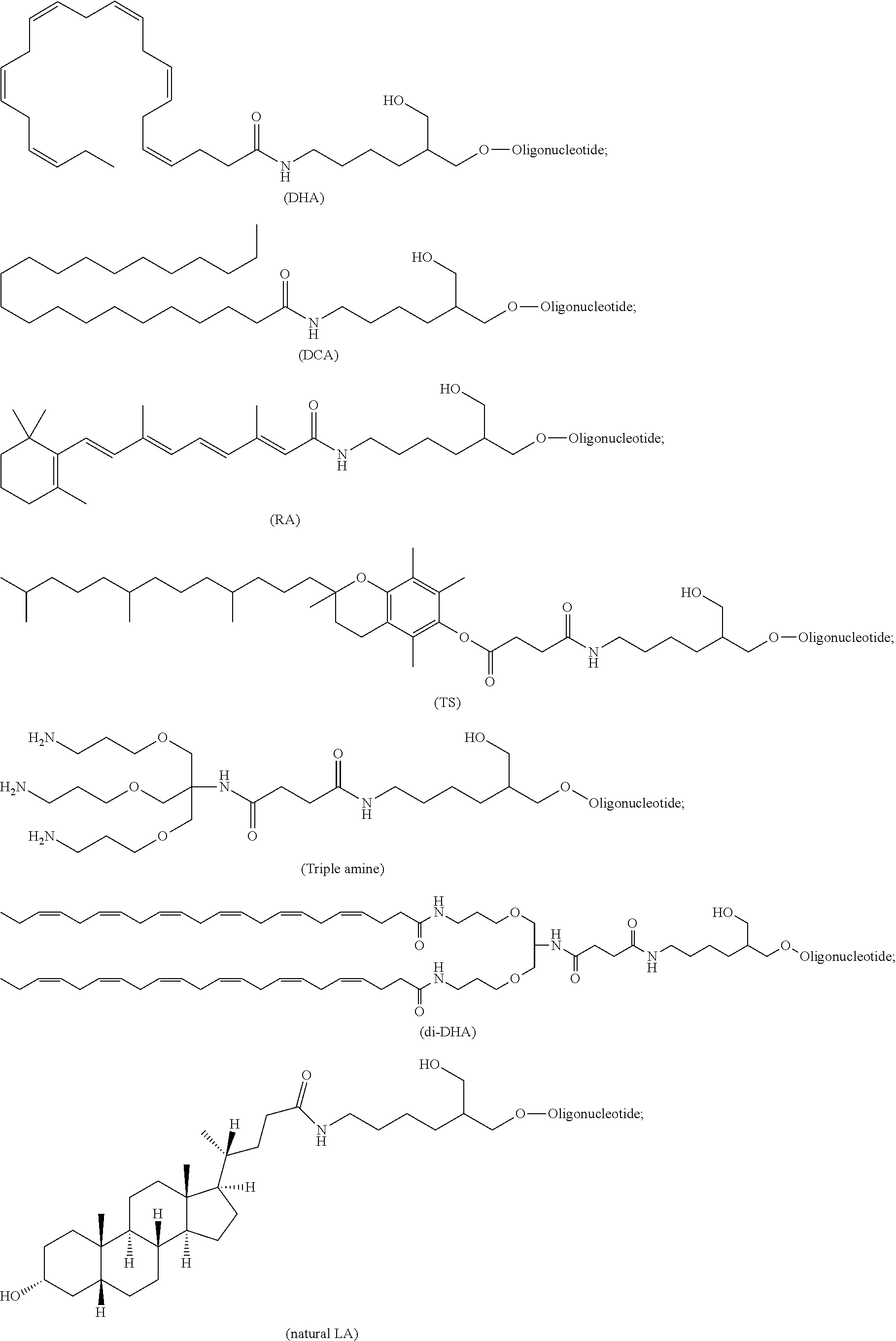

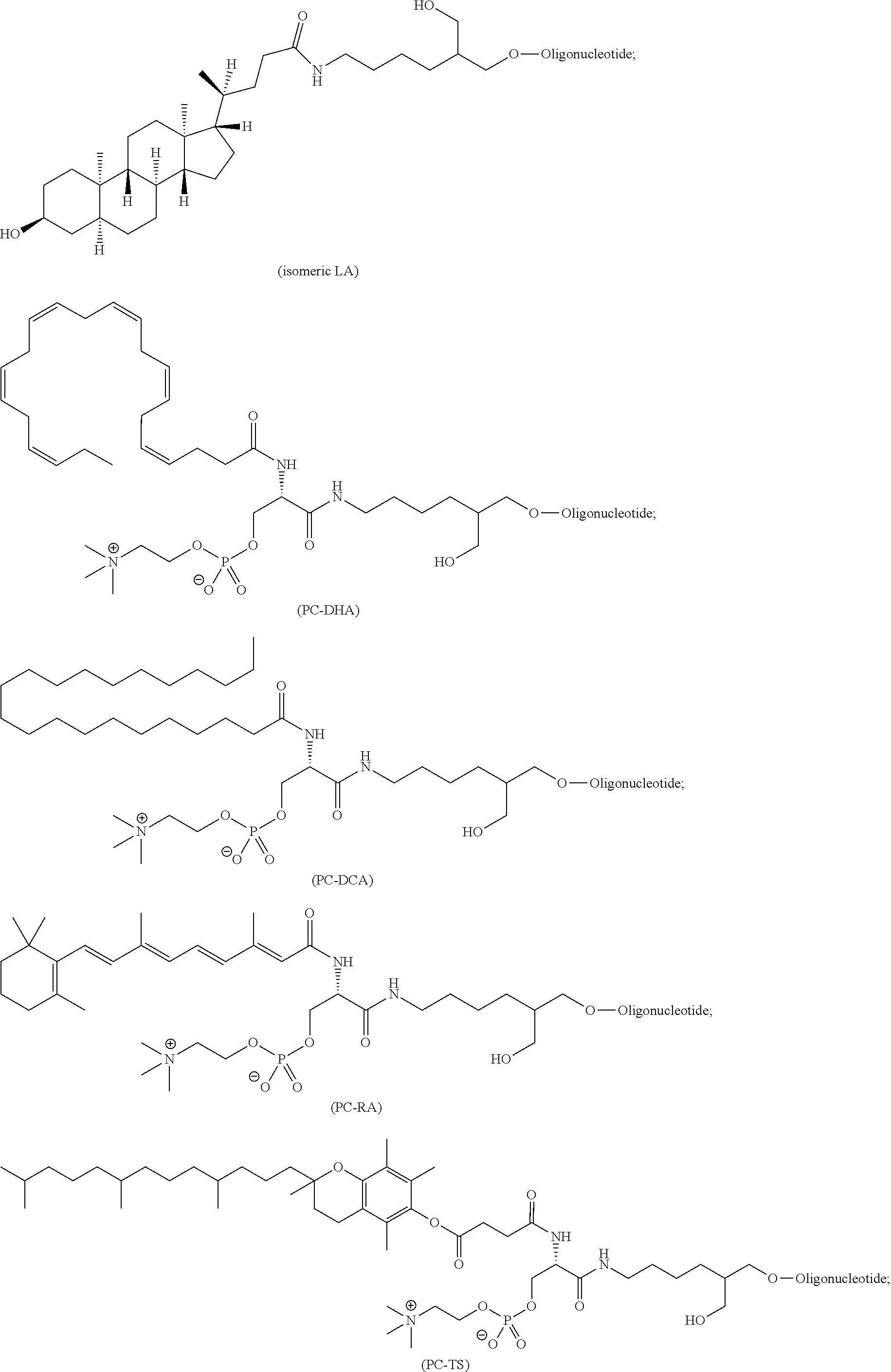

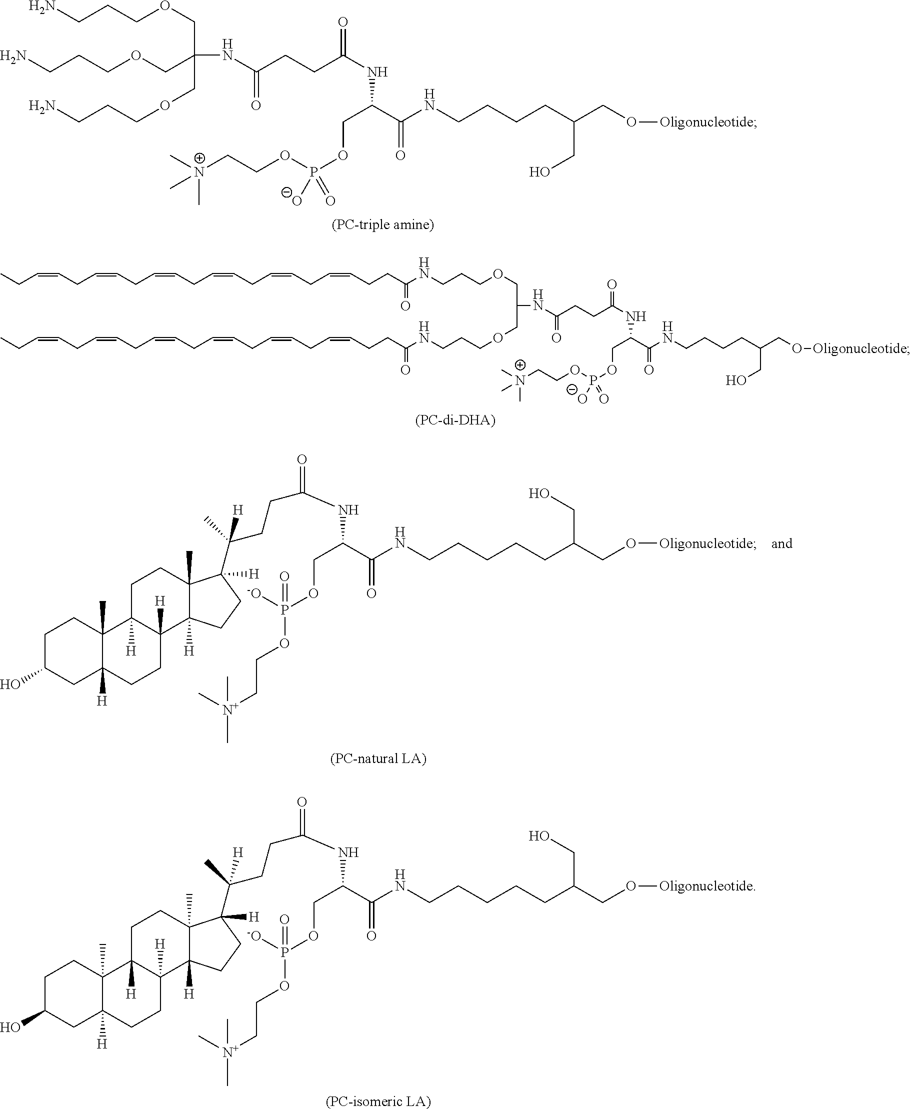

For any of the above recited structures, the term “oligonucleotide” corresponds to any of the oligonucleotides recited herein, e.g., an ASO or siRNA. In certain embodiments, the term “oligonucleotide” in the structures recited above corresponds to the sense strand of an siRNA. In certain embodiments, the oxygen immediately adjacent to the term “oligonucleotide” in the structures is linked to the 3′ end of a sense strand of an siRNA.

### Di-DHA Oligonucleotide Conjugate

In one aspect, the disclosure provides an oligonucleotide conjugate comprising: i) an oligonucleotide comprising a 5′ and a 3′ end and complementarity to a target nucleic acid; and ii) a di-docosahexaenoic acid (di-DHA) functional moiety linked to the oligonucleotide.

In certain embodiments, the di-DHA functional moiety is phosphatidylcholine (PC) esterified di-DHA (PC-di-DHA).

In certain embodiments, the oligonucleotide conjugate comprises the structure:

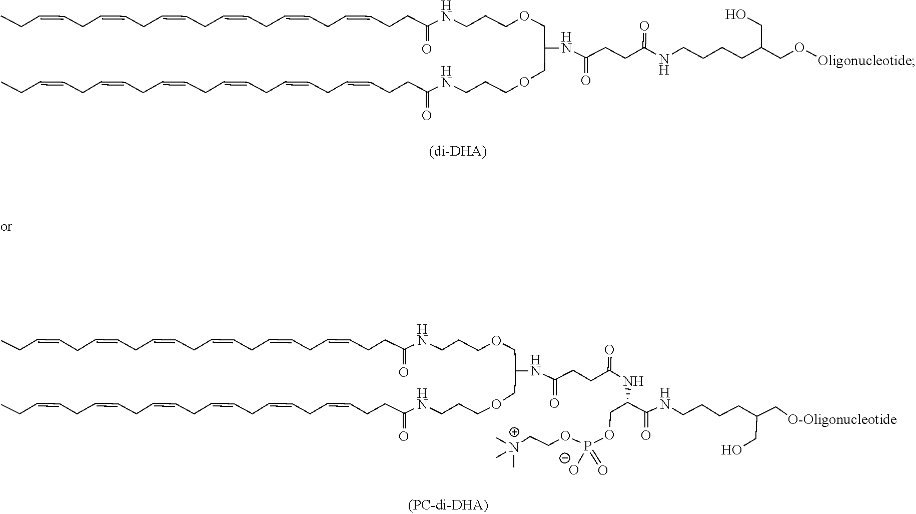

In certain embodiments, the oligonucleotide corresponds to an antisense oligonucleotide or a siRNA.

In certain embodiments, the siRNA comprises a sense strand and an antisense strand.

In certain embodiments, the functional moiety (i.e., the di-DHA or PC-di-DHA) is linked to the 5′ end and/or 3′ end of the sense strand or to the 5′ end and/or 3′ end of the antisense strand. In certain embodiments, the functional moiety is linked to the 3′ end of the sense strand.

### Triple Amine Oligonucleotide Conjugate

In one aspect, the disclosure provides an oligonucleotide conjugate comprising: i) an oligonucleotide comprising a 5′ and a 3′ end and complementarity to a target nucleic acid; and ii) a triple amine functional moiety linked to the oligonucleotide.

In certain embodiments, the triple amine functional moiety is phosphatidylcholine (PC) esterified triple amine (PC-triple amine).

In certain embodiments, the oligonucleotide conjugate comprises the structure:

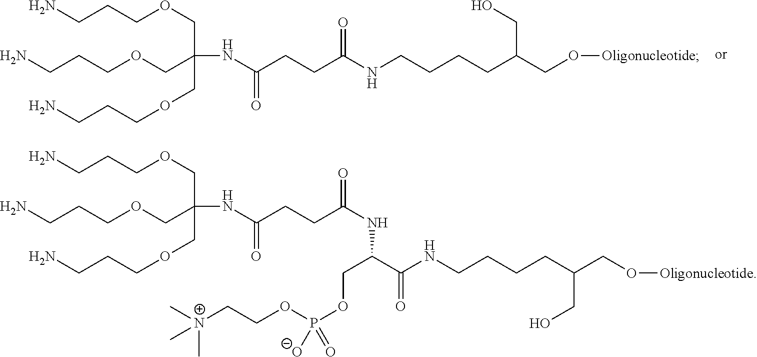

In certain embodiments, the oligonucleotide corresponds to an antisense oligonucleotide or a siRNA.

In certain embodiments, the siRNA comprises a sense strand and an antisense strand.

In certain embodiments, the functional moiety (i.e., the triple amine or PC-triple amine) is linked to the 5′ end and/or 3′ end of the sense strand or to the 5′ end and/or 3′ end of the antisense strand. In certain embodiments, the functional moiety is linked to the 3′ end of the sense strand.

### Branched Oligonucleotides

The branched oligonucleotides described here comprise two or more oligonucleotides linked together. The different branched oligonucleotides described herein (e.g., a branched oligonucleotide with two, three, or four oligonucleotides) enhanced eye delivery of the oligonucleotide, including eye cell-specific delivery.

In one aspect, the disclosure provides a method for delivering a branched oligonucleotide to an eye of a subject, the method comprising administering the branched oligonucleotide to the subject, wherein the branched oligonucleotide comprises two or more oligonucleotides, each oligonucleotide comprising a 5′ and a 3′ end and complementarity to a target nucleic acid.

In certain embodiments, one or more of the oligonucleotides of the branched oligonucleotide further comprises a functional moiety linked to the oligonucleotide, wherein the functional moiety comprises any one of triple amine, retinoic acid, DHA, DCA, α-tocopheryl succinate, or LA. The functional moieties as described above in the Oligonucleotide Conjugate section can be applied to the oligonucleotides of the branched oligonucleotides. Similarly, the oligonucleotides as described above in the Oligonucleotide Conjugate section can serve as the oligonucleotides of the branched oligonucleotides, including type (ASO or siRNA), strand length, and chemical modifications.

In certain embodiments, the two or more oligonucleotides in the branched oligonucleotide are connected to one another by one or more moieties independently selected from a linker, a spacer and a branching point.

In certain embodiments, the linker comprises an ethylene glycol chain, an alkyl chain, a peptide, an RNA, a DNA, a phosphate, a phosphonate, a phosphoramidate, an ester, an amide, a triazole, or combinations thereof.

In certain embodiments, the branching point comprises a polyvalent organic species or derivative thereof.

In another embodiment, the branching point is an amino acid derivative. In another embodiment of the branching point is selected from the formulas of:

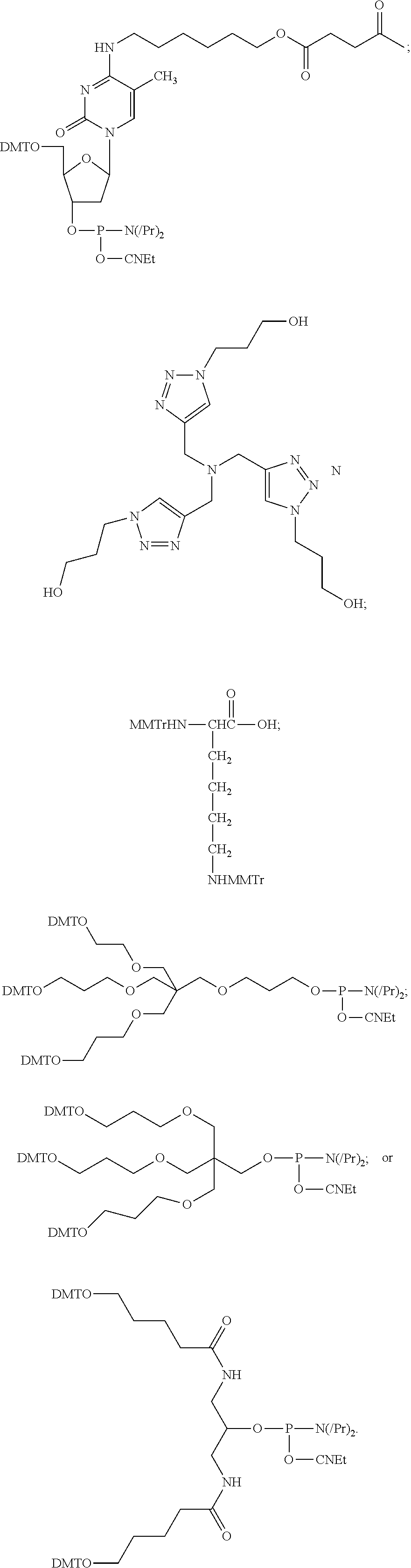

Polyvalent organic species are moieties comprising carbon and three or more valencies (i.e., points of attachment with moieties such as S, L or N, as defined above). Non-limiting examples of polyvalent organic species include triols (e.g., glycerol, phloroglucinol, and the like), tetrols (e.g., ribose, pentaerythritol, 1,2,3,5-tetrahydroxybenzene, and the like), tri-carboxylic acids (e.g., citric acid, 1,3,5-cyclohexanetricarboxylic acid, trimesic acid, and the like), tetra-carboxylic acids (e.g., ethylenediaminetetraacetic acid, pyromellitic acid, and the like), tertiary amines (e.g., tripropargylamine, triethanolamine, and the like), triamines (e.g., diethylenetriamine and the like), tetramines, and species comprising a combination of hydroxyl, thiol, amino, and/or carboxyl moieties (e.g., amino acids such as lysine, serine, cysteine, and the like).

In certain embodiments, the spacer comprises an ethylene glycol chain, an alkyl chain, a peptide, an RNA, a DNA, a phosphate, a phosphonate, a phosphoramidate, an ester, an amide, a triazole, or a combination thereof.

In certain embodiments, the linker comprises the structure L1:

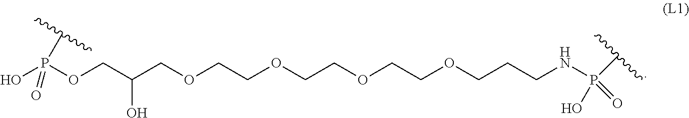 [SMILES: *P(=O)(O)NCCCOCCOCCOCCOCC(O)COP(*)(=O)O]

In certain embodiments, the linker comprises the structure L2:

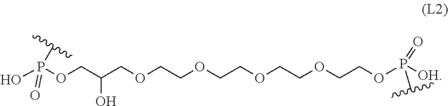 [SMILES: *P(=O)(O)OCCOCCOCCOCCOCC(O)COP(*)(=O)O]

In certain embodiments, the branched oligonucleotide consists of two oligonucleotides. In certain embodiments, the branched oligonucleotide consists of three oligonucleotides. In certain embodiments, the branched oligonucleotide consists of four oligonucleotides. In certain embodiments, the oligonucleotides are siRNA.

In certain embodiments, the branched oligonucleotide comprises the structure:

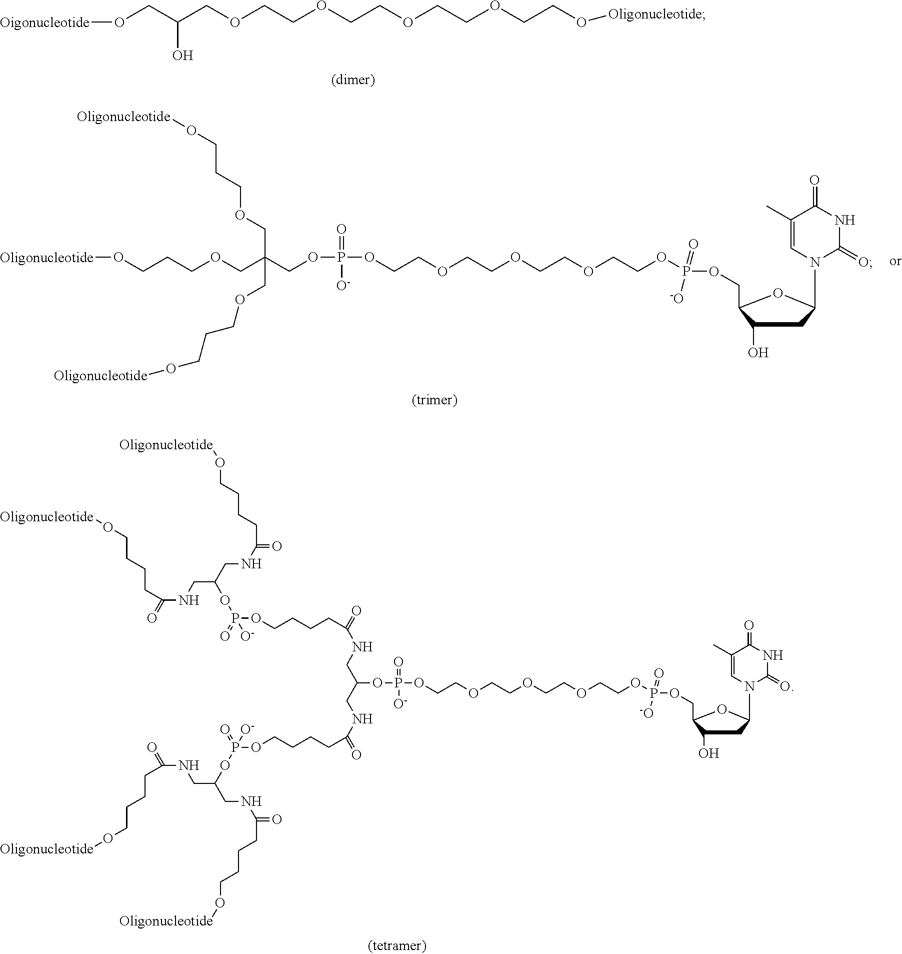

For any of the above recited structures, the term “oligonucleotide” corresponds to any of the oligonucleotides recited herein, e.g., an ASO or siRNA. In certain embodiments, the term “oligonucleotide” in the structures recited above corresponds to the sense strand of an siRNA. In certain embodiments, the oxygen immediately adjacent to the term “oligonucleotide” in the structures is linked to the 3′ end of a sense strand of an siRNA.

Branched oligonucleotides, including synthesis and methods of use, are described in greater detail in WO2017/132669, incorporated herein by reference. Further details regarding synthesis are provided in the Materials and Methods section of the Examples.

### Methods of Delivery to Eye Cells

The oligonucleotide conjugates and branched oligonucleotides described herein are capable of eye-cell specific delivery with effective silencing of a target gene. Any given oligonucleotide conjugate and branched oligonucleotide may be effective at delivery to more than one type of eye cell.

In certain embodiments, the oligonucleotide conjugate or branched oligonucleotide is administered by intravitreal injection.

In certain embodiments, the oligonucleotide conjugate or branched oligonucleotide is delivered to an eye cell after administration to a subject.

In certain embodiments, the eye cell is selected from the group consisting of a Müller glia cell, a rod photoreceptor cell, a cone photoreceptor cell, a ganglion cell, an amacrine cell, a bipolar cell, and a horizontal cell.

In certain embodiments, the eye cell is selected from the group consisting of a glutamine synthetase (GS)-expressing eye cell, a rhodopsin-expressing eye cell, a cone arrestin (CA)-expressing eye cell, a Vglut2-expressing eye cell, a VGAT-expressing eye cell, a protein kinase C alpha (PKCa)-expressing eye cell, and a Lim1-expressing eye cell.

In certain embodiments, the oligonucleotide conjugate has selective affinity for a retinal protein.

In certain embodiments, the eye cell is a Müller glia cell, and: i) the oligonucleotide conjugate comprises DHA, DCA, α-tocopheryl succinate, or LA; or ii) the branched oligonucleotide consists of two, three, or four oligonucleotides. In certain embodiments of Müller glia cell delivery, the DHA, α-tocopheryl succinate, and LA are phosphatidylcholine (PC) esterified DHA (PC-DHA), α-tocopheryl succinate (PC-TS), and LA (PC-LA).

In certain embodiments, the eye cell is a rod photoreceptor cell, and: i) the oligonucleotide conjugate comprises DCA; or ii) the branched oligonucleotide consists of three or four oligonucleotides.

In certain embodiments, the eye cell is a cone photoreceptor cell, and: i) the oligonucleotide conjugate comprises retinoic acid, DHA, DCA, α-tocopheryl succinate, or LA; or ii) the branched oligonucleotide consists of two, three or four oligonucleotides. In certain embodiments of cone photoreceptor cell delivery, the retinoic acid and α-tocopheryl succinate are phosphatidylcholine (PC) esterified retinoic acid (PC-RA) and α-tocopheryl succinate (PC-TS). In certain embodiments of cone photoreceptor cell delivery, the oligonucleotide conjugate comprises two DHA functional moieties.

In certain embodiments, the eye cell is a ganglion cell, and the oligonucleotide conjugate comprises α-tocopheryl succinate. In certain embodiments of the ganglion cell delivery, the α-tocopheryl succinate is phosphatidylcholine (PC) esterified α-tocopheryl succinate (PC-TS).

In certain embodiments, the eye cell is an amacrine cell, and: i) the oligonucleotide conjugate comprises retinoic acid, DHA, DCA, α-tocopheryl succinate, or LA; or ii) the branched oligonucleotide consists of two, three or four oligonucleotides. In certain embodiments of the amacrine cell delivery, the retinoic acid, DHA, α-tocopheryl succinate, and LA are phosphatidylcholine (PC) esterified retinoic acid (PC-RA), DHA (PC-DHA), α-tocopheryl succinate (PC-TS), and LA (PC-LA). In certain embodiments of the amacrine cell delivery, the oligonucleotide conjugate comprises two DHA functional moieties or two PC-DHA functional moieties.

In certain embodiments, the eye cell is a bipolar cell, and: i) the oligonucleotide conjugate comprises triple amine, retinoic acid, DHA, DCA, α-tocopheryl succinate, or LA; or ii) the branched oligonucleotide consists of two, three or four oligonucleotides. In certain embodiments of the bipolar cell delivery, the retinoic acid, DHA, α-tocopheryl succinate, and LA are phosphatidylcholine (PC) esterified retinoic acid (PC-RA), DHA (PC-DHA), α-tocopheryl succinate (PC-TS), and LA (PC-LA). In certain embodiments of the bipolar cell delivery, the oligonucleotide conjugate comprises two DHA functional moieties or two PC-DHA functional moieties.

In certain embodiments, the eye cell is a horizontal cell, and: i) the oligonucleotide conjugate comprises DCA; or ii) the branched oligonucleotide consists of two oligonucleotides.

### Methods of Gene Silencing/Methods of Treatment

The oligonucleotide conjugates and branched oligonucleotides described herein are capable of target gene (i.e., target nucleic acid) silencing in the eye and within specific eye cells.

In certain embodiments, expression of the target nucleic acid is reduced by at least 20%, at least 30%, at least 40%, or at least 50% in an eye cell.

In certain embodiments, expression of the target nucleic acid is reduced by at least 20%, at least 30%, at least 40%, or at least 50% in an eye cell selected from the group consisting of a Müller glia cell, a rod photoreceptor cell, a cone photoreceptor cell, a ganglion cell, an amacrine cell, a bipolar cell, and a horizontal cell.

In certain embodiments, expression of the target nucleic acid is reduced by at least 20%, at least 30%, at least 40%, or at least 50% in an eye cell selected from the group consisting of a glutamine synthetase (GS)-expressing eye cell, a rhodopsin-expressing eye cell, a cone arrestin (CA)-expressing eye cell, a Vglut2-expressing eye cell, a VGAT-expressing eye cell, a protein kinase C alpha (PKCa)-expressing eye cell, and a Lim1-expressing eye cell.

In one aspect, the disclosure provides a method of treating an eye disorder in a subject in need thereof, the method comprising administering the oligonucleotide conjugate and/or branched oligonucleotide described herein to the subject, thereby treating the eye disorder.

In certain embodiments, administration of the oligonucleotide conjugate or the branched oligonucleotide results in the treatment of an eye disorder in the subject.

In certain embodiments, administration of the oligonucleotide conjugate or the branched oligonucleotide results in the reduction of gene expression from the target nucleic acid that is associated with the eye disorder in the subject.

In certain embodiments, the oligonucleotide conjugate or branched oligonucleotide is administered by intravitreal injection.

In certain embodiments, the eye disorder is selected from the group consisting of age-related macular degeneration, diabetic retinopathy, central cataract, normal-tension glaucoma, macular edema, and glaucoma.

### EXAMPLES

### Materials and Methods

### Synthesis of Lipid Functionalized Solid Supports

Non-phosphocholine (PC) lipid moieties (except α-tocopheryl succinate) were directly attached via a peptide bond to a controlled pore glass (CPG) functionalized by a C7 linker, as described previously (Nikan M, Osborn M F, Coles A H, et al. Docosahexaenoic acid conjugation enhances distribution and safety of siRNA upon local administration in mouse brain. Mol. Ther. Nucleic Acids. 2016; 5:e344). To synthesize PC derivatives, amino C7 CPG was first functionalized with phosphocholine (Nikan M, Osborn M F, Coles A H, et al. Synthesis and evaluation of parenchymal retention and efficacy of a metabolically stable O-Phosphocholine-N-docosahexaenoyl-l-serine siRNA conjugate in mouse brain. Bioconjug. Chem. 2017; 28:758-1766). Briefly, Fmoc-L-serine tert-butyl (TCI America) was phosphitylated using 2′-cyanoethyl-N,N-diisopropylchlorophosphoramidite (ChemGenes). The resulting phosphoramidite was coupled to choline p-toluenesulfonate (Alfa Aesar) using 5-(ethylthio)-1H-tetrazole (ETT) as an activator. The phosphine ester was then oxidized, and the carboxylic acid and phosphate ester groups were deprotected (i.e., tert-butyl and cyanoethyl groups removed). The resulting intermediate was attached to the amino C7 CPG via a peptide bond to form phosphocholine-functionalized CPG. The Fmoc group was removed, and the selected lipid moiety was attached via a peptide bond to the CPG. All lipid-functionalized solid supports were obtained with a loading of 55 μmol/g.

### Synthesis of α-Tocopheryl Succinate-Conjugated Oligonucleotides

α-tocopheryl succinate was attached to the amino group at the 3′end of the purified oligocnucleotide synthesized on amino C7 CPG or phosphocholine-functionalized amino C7 CPG. N-hydroxysuccinimide α-tocopheryl succinate and purified oligonucleotides were combined in a solution of 0.1 M sodium bicarbonate, 20% (v/v) dimethylformamide and incubated overnight at room temperature. One-tenth volume of 3M sodium acetate (pH 5.2) was added to obtain a final concentration of 0.3M sodium acetate. Three volumes 95% (v/v) ethanol were added, and the mixture was vortexed and then placed for 1 h at −80° C. The solution was pelleted by centrifugation for 30 min at 5200×g. The pellet containing the lipid-conjugated siRNA sense strand was dissolved in water, purified, and desalted as described below.

### Oligonucleotide Synthesis

Oligonucleotides were synthesized by phosphoramidite solid-phase synthesis on a Dr Oligo 48 (Biolytic, Fremont, CA) or MerMade12 (Biosearch Technologies, Novato, CA), using 2′-F or 2′-O-Me modified phosphoramidites with standard protecting groups. 5′-(E)-Vinyl tetra phosphonate (pivaloyloxymethyl) 2′-O-methyl-uridine 3′-CE phosphoramidite (VP) for in vivo unconjugated oligonucleotides was purchased from Hongene Biotech, USA, Quasar 570 CE phosphoramidite (Cy3) was purchased from GenePharma, Shanghai, China. Bis-cyanoethyl-N,N-diisopropyl CED phosphoramidite (5′P) for in vitro unconjugated oligonucleotides and all other phosphoramidites used were purchased from ChemGenes, Wilmington, MA. Phosphoramidites were prepared at 0.1 M in anhydrous acetonitrile (ACN), except for 2′-O-methyl-uridine phosphoramidite dissolved in anhydrous ACN containing 15% dimethylformamide. 5-(Benzylthio)-1H-tetrazole (BTT) was used as the activator at 0.25 M, and the coupling time for all phosphoramidites was 4 min, using 10 eq. Detritylations were performed using 3% trichloroacetic acid in dichloromethane. Capping reagents used were CAP A (20% n-methylimidazole in ACN) and CAP B (20% acetic anhydride and 30% 2,6-lutidine in ACN). Reagents for capping and detritylation were purchased from AIC, Framingham, MA. Phosphite oxidation to convert to phosphate or phosphorothioate was performed with 0.05 M iodine in pyridine-H₂O (9:1, v/v) or 0.1 M solution of 3-[(dimethylaminomethylene)amino]-3H-1,2,4-dithiazole-5-thione (DDTT) in pyridine (ChemGenes) for 4 min. Unconjugated oligonucleotides were synthesized on 500 Å long-chain alkyl amine (LCAA) controlled pore glass (CPG) functionalized with Unylinker terminus (ChemGenes). Cholesterol conjugated oligonucleotides were synthesized on a 500 Å LCAA-CPG support, where the cholesterol moiety is bound to tetra-ethylenglycol through a succinate linker (ChemGenes, Wilmington, MA). Lipid conjugated oligonucleotides were synthesized on modified solid support (synthesis described above). Divalent oligonucleotides (Dimer) were synthesized on modified solid support, synthesis described previously (Alterman J F, Godinho B M D C, Hassler M R, et al. A divalent siRNA chemical scaffold for potent and sustained modulation of gene expression throughout the central nervous system. Nat Biotechnol 37, 884-894 (2019)).

### Branched Oligonucleotides Synthesis

Synthesis of branched oligonucleotides was performed by phosphoramidite solid-phase, on an AKTA Oligoplilot 10 (Cytiva, Marlborough, MA) with the parameters described above, or otherwise specified here. Trimer and Tetramer branched oligonucleotides were prepared using commercial trebler and doubler phosphoramidites respectively purchased from Glen Research, Sterling, VA. Trimer linker was produced in two steps as follows; on a 1000 Å Thymidine 3′-LCAA-CPG (ChemGenes), DMT-tetraethyloxy-Glycol CED phosphoramidite (ChemGenes) was coupled first for 8 min, using 10 eq, followed by the coupling of the trebler phosphoramidite for 8 min, using 10 eq. Trivalent oligonucleoides were grown afterwards on this linker using 30 eq. Tetramer linker was produced in three steps as follows; first, on a 1000 Å Thymidine 3′-LCAA-CPG, DMT-tetraethyloxy-Glycol CED phosphoramidite was coupled for 8 min, using 10 eq, second, the coupling of doubler phosphoramidite for 8 min, using 10 eq, and third, a subsequent coupling of doubler phosphoramidite for 8 min, using 20 eq was performed. Tetravalent oligonucleotides were grown afterwards using 40 eq.

### Deprotection and Purification of Oligonucleotides for Screening of Sequences

Prior to the deprotection, synthesis columns containing oligonucleotides were treated with 10% diethylamine (DEA) in ACN to deprotect cyanoethyl groups. In synthesis columns, both unconjugated and cholesterol conjugated oligonucleotides on solid support were then deprotected with methylamine gas (Airgas) for an hour at room temperature. Deprotected oligonucleotides released from the solid support were precipitated on the support by passing solution of (i) a mixture of 0.1 M sodium acetate in 85% ethanol and then (ii) 85% ethanol to the synthesis column. The excess ethanol on solid support was dried by air flow and the oligonucleotides were flushed out by passing water through the column. This procedure renders pure oligonucleotides used for in vitro experiments.

### Deprotection and Purification of Oligonucleotides for In Vivo Experiments

Prior to the deprotection, synthesis columns containing oligonucleotides were treated with 10% diethylamine (DEA) in ACN to deprotect cyanoethyl groups. Cy3 labeled and lipid conjugated oligonucleotides were cleaved and deprotected in 28-30% ammonium hydroxide, 40% aq. methylamine (1:1, v/v) (AMA) for 2 h at room temperature. Cy3 labeled and non-labeled unconjugated, divalent, trivalent, and tetravalent oligonucleotides were cleaved and deprotected by AMA treatment at 45° C. for 2 h. The VP containing oligonucleotides did not have a pretreatment with DEA post-synthesis and were cleaved, and deprotected as described previously (O'Shea J, Theile C S, Das R, et al, An efficient deprotection method for 5′-[O,O-bis(pivaloyloxymethyl)]-(E)-vinylphosphonate containing oligonucleotides. Tetrahedron 74, 6182-6186 (2018)). Briefly, CPG with VP-oligonucleotides was treated with a solution of 3% DEA in 28-30% ammonium hydroxide at 35° C. for 20 hours.

All solutions containing cleaved oligonucleotides were filtered to remove the CPG and dried under vacuum. The resulting pellets were re-suspended in 5% ACN in water. Purifications were performed on an Agilent 1290 Infinity II HPLC system. VP and non-labeled unconjugated, divalent, trivalent and tetravalent oligonucleotides were purified using a custom 25×150 mm column packed with Source 15Q anion exchange resin (Cytiva, Marlborough, MA); running conditions: eluent A, 10 mM Tris-HCl buffer (pH 9) in 7.5% ACN in water; eluent B, 1 M sodium perchlorate in 10 mM Tris-HC buffer (pH 9) in 7.5% ACN in water; linear gradient, 12 to 35% B in 40 min at 50° C. Lipid conjugated and Cy3 labeled oligonucleotides were purified using a 21.2×150 mm PRP-C18 column (Hamilton Co, Reno, NV); running conditions: eluent A, 50 mM sodium acetate (pH 6) in 5% ACN in water; eluent B, 100% ACN; linear gradient, 15 to 60% B in 40 min at 60° C. Flow was 40 mL/min in both methods and peaks were monitored at 260 nm for non-labeled oligonucleotides and 550 nm for labeled oligonucleotides. A separate column was used for Cy3 labeled oligonucleotides to avoid cross-contamination. Fractions were analyzed by liquid chromatography mass spectrometry (LC-MS), pure fractions combined and dried under vacuum. Oligonucleotides were re-suspended in 5% ACN and desalted by size exclusion on a 25×250 mm custom column packed with Sephadex G-25 media (Cytiva, Marlborough, MA), using isocratic method with HPLC grade water (Honeywell Chemicals, Charlotte, NC) and finally oligonucleotides were lyophilized.

### LC-MS Analysis of Oligonucleotides

The identity of oligonucleotides was verified by LC-MS analysis on an Agilent 6530 accurate mass Q-TOF using the following conditions: buffer A: 100 mM 1,1,1,3,3,3-hexafluoroisopropanol (HFIP) and 9 mM triethylamine (TEA) in LC-MS grade water; buffer B:100 mM HFIP and 9 mM TEA in LC-MS grade methanol; column, Agilent AdvanceBio oligonucleotides C18; linear gradient 0-40% B 5 min (Unconjugated, divalent, trivalent and tetravalent oligonucleotides); linear gradient 50-100% B 5 min (Lipid conjugated and Cy3 labeled oligonucleotides); temperature, 60° C.; flow rate, 0.85 ml/min. LC peaks were monitored at 260 nm and for labeled oligonucleotides at 550 nm. MS parameters: Source, electrospray ionization; ion polarity, negative mode; range, 100-3,200 m/z; scan rate, 2 spectra/s; capillary voltage, 4,000; fragmentor, 200 V; gas temp, 325° C.

### In Vivo Experiments

Intravitreal injections into adult mice were performed as previously described (Venkatesh A, Ma S, Langellotto F, et al. Retinal gene delivery by rAAV and DNA electroporation. Curr Protoc Microbio: 2013; Chapter 14:Unit 14D 14.). Injections were performed with glass needles (Clunbury Scientific LLC; Cat no. B100-58-50) using the FemtoJet from Eppendorf with a constant pressure and injection time of 300 psi and 1.5 s, respectively, to deliver ˜2 mL of fluid into the vitreous. All concentrations were adjusted to use a 2 mL injection volume for the desired amount of siRNA. Intravitreal injections into adult pigs used an Insulin injection needle to inject 100 mL of siRNA ˜2-3 mm from the temporal limbus into the vitreous. Anesthesia and euthanasia of pigs was performed by animal medicine according to standard procedures. Cornea was treated with proparacaine and ophthalmic Betadine before injection of the siRNA. After injection eyes were rinsed with saline eye wash solution. Enucleated pig and mouse eyes were processed as described (Venkatesh A, Ma S, Langellotto F, et al. Retinal gene delivery by rAAV and DNA electroporation. Curr Protoc Microbio: 2013; Chapter 14:Unit 14D 14).

### Example 1: Delivery of siRNA for Eye Diseases

An initial screen of different siRNA, all targeting Htt gene with the same sequence, was conducted in order to study their distribution and efficiency on knockdown in the eye.

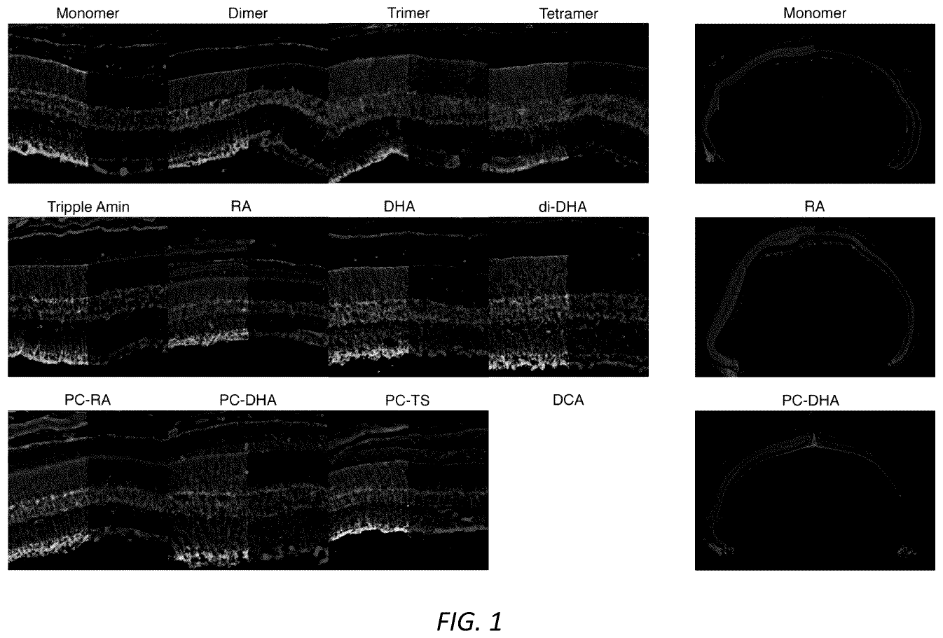

The cellular distribution is difficult to ascertain from the Cy3 label alone. Therefore, a lower dose of the siRNA compounds (0.1 nanomole) was injected (siRNAs labeled with Cy3 are shown in red in FIG. 1). 3 days later, the tissue was dissociated, Cy3 positive cells were FACS sorted, and cell type specific antibodies were used to determine which cell types were enriched by which siRNA modification.

FIG. 1 shows all siRNAs are labeled with Cy3 and in red, meanwhile glutamine synthetase (GS) expression, which is specific to Müller glia cells, is shown in green, and nuclear DAPI is shown in blue. All siRNA can be seen across the entire retinal cross section with slightly different cellular distribution. The right of FIG. 1 displays one example per group showing entire retinal cross section with distribution across the entire retina: half of the section shows nuclear DAPI, GS and the siRNA, the other half only the siRNA.

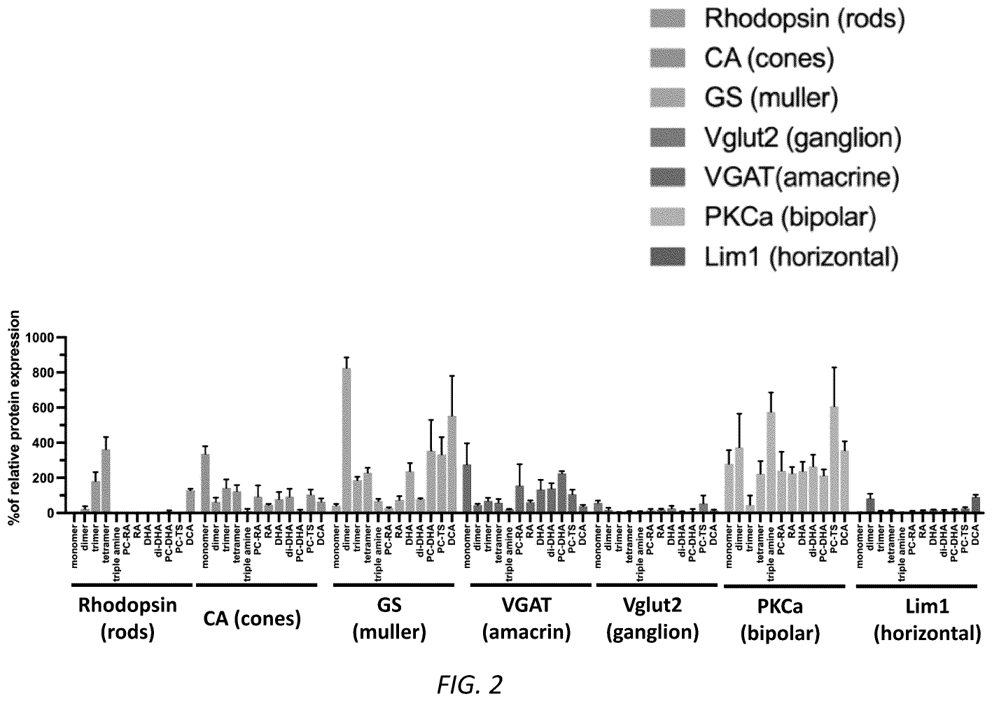

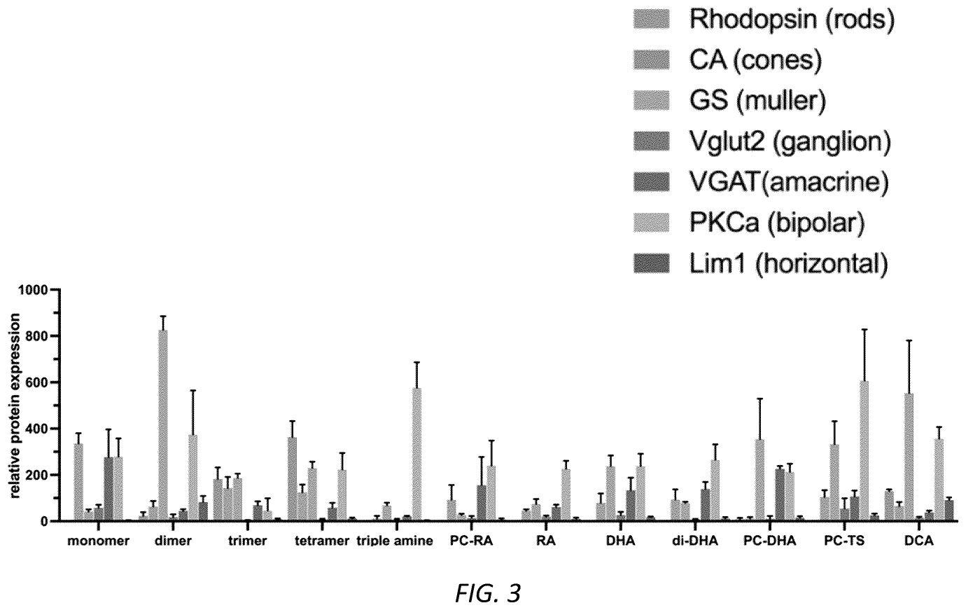

The overall goal was A) to determine the cellular distribution and B) to identify the modifications that allow for best cell entry into cone and rod photoreceptors and Müller glia cells, as these three cell types are the most important ones to target for many retinal diseases. FIGS. 2 and 3 show the outcome of the enrichment of siRNA in different retinal cell types arranged by cell type (FIG. 2) and by modification (FIG. 3). The findings suggest that the Monomer configuration is good for cone photoreceptors, the tetramer is good for rod photoreceptors and the dimer is best for Müller glia cells. Other good Müller glia cell compounds are PC-TS, PC-DHA and DCA.

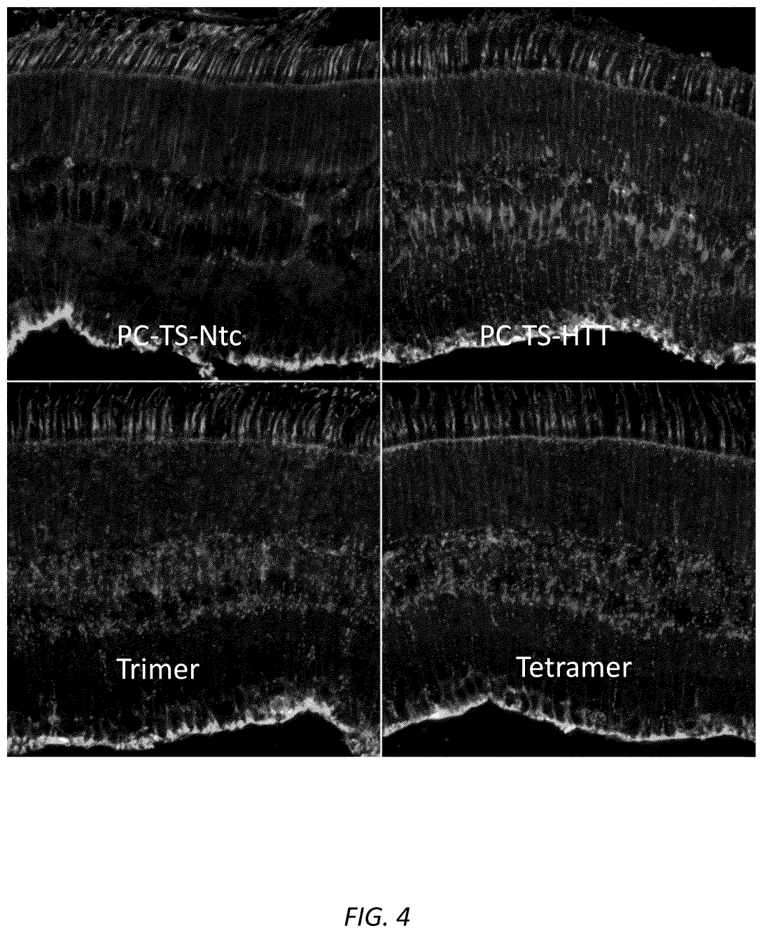

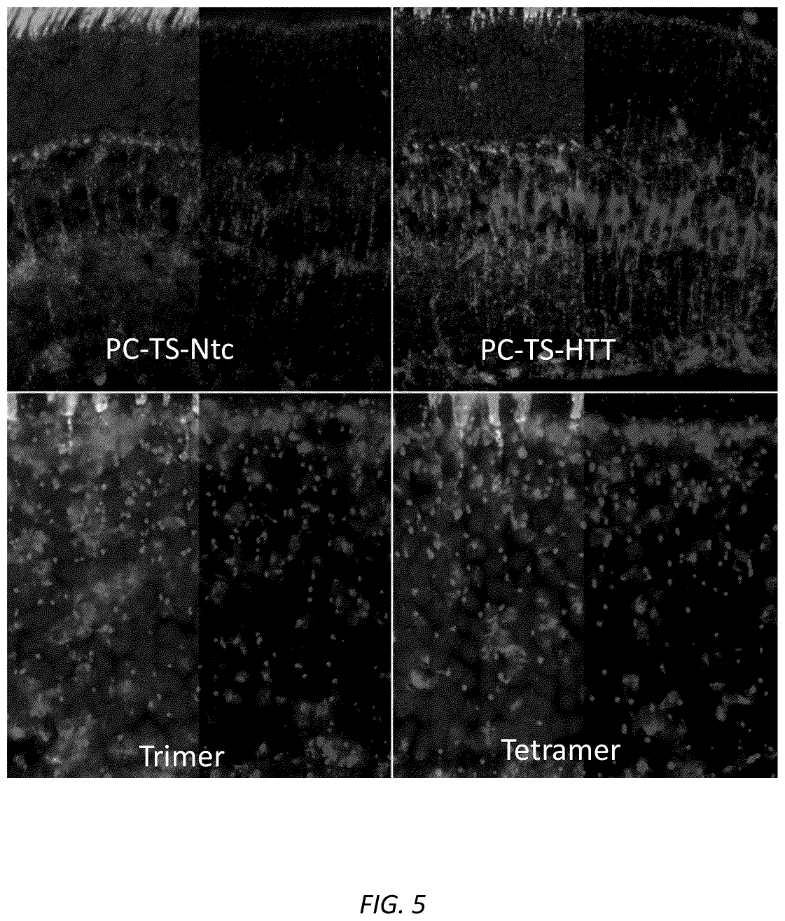

Repeat injections and higher magnifications of a subset of these compounds show that PC-TS accumulates well in Müller Glia cells and the trimer and tetramer accumulates well in rod photoreceptors (FIGS. 4 and 5).

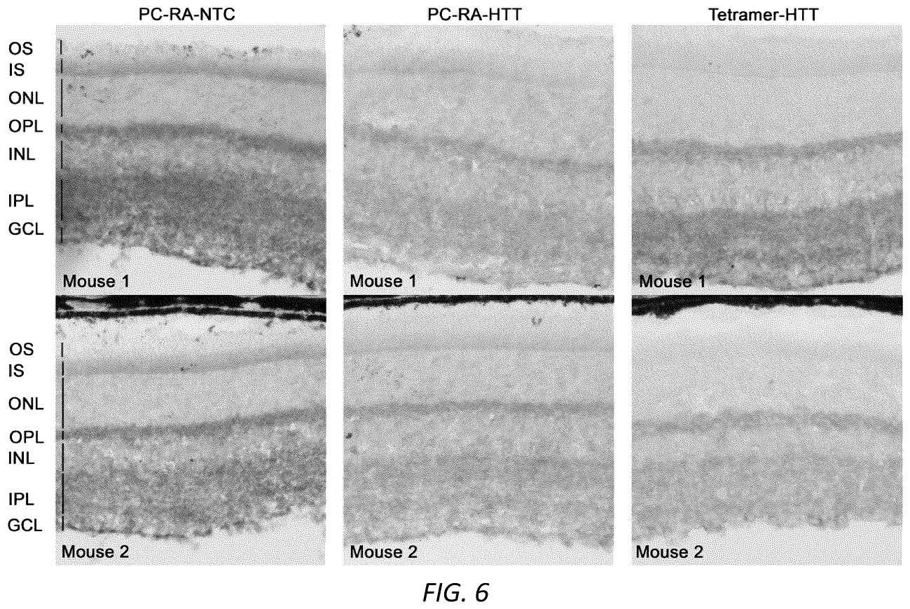

To determine if the tetramer is able to knockdown HTT protein better in photoreceptors than other configurations, an antibody staining was performed against the HTT protein two weeks after intravitreal injection of 0.3 nanomoles of the siRNAs shown in FIG. 6. First column in FIG. 6 shows antibody staining in control mice injected with the NTC-siRNA. HTT protein has a pan-retinal expression with particular enrichment in the photoreceptor inner segments (IS), in the outer plexiform layer (OPL) where photoreceptors make synaptic connections with the bipolar cells and the horizontal cells, an intermediate enrichment in the inner nuclear layer (INL), where bipolar cell, amacrine cell, horizontal cell and Mueller glia cell bodies reside, and a strong enrichment in the inner plexiform layer (IPL) where synaptic connections of amacrine, bipolar and ganglion cells reside. Second column, expression of HTT protein after knockdown with PC-RA-Htt siRNA. This siRNA tends to accumulate preferentially in bipolar and amacrine cells, as shown in FIGS. 2 & 3. Thus, expression in the OPL and IPL are reduced more efficiently with this siRNA. Third column shows expression of HTT protein in 2 different mice each injected with ˜0.3 nanomoles of the Htt-siRNA after knockdown with Tetramer-Htt siRNA. This configuration tends to accumulate efficiently in rod photoreceptors and bipolar cells. Consistent with that expression in photoreceptor ISs is reduced much more than with the PC-RA-Htt siRNA.

Quantification by Western Blotting of total HTT protein remaining from total retinal extracts was also performed two weeks after injection with the Htt-siRNA using the same experimental setting done is FIG. 6 using different mice from the same injected batch. Note that the knockdown is at about 50% relative expression at 2 weeks post injection when quantifying total HTT protein. The two configurations targeted the different cell population of the retina at different efficiencies. Nonetheless, the overall knockdown is similar due to the ubiquitous expression of HTT in the retina.

### Example 2: Assessment of Htt mRNA Knockdown by bDNA Assays

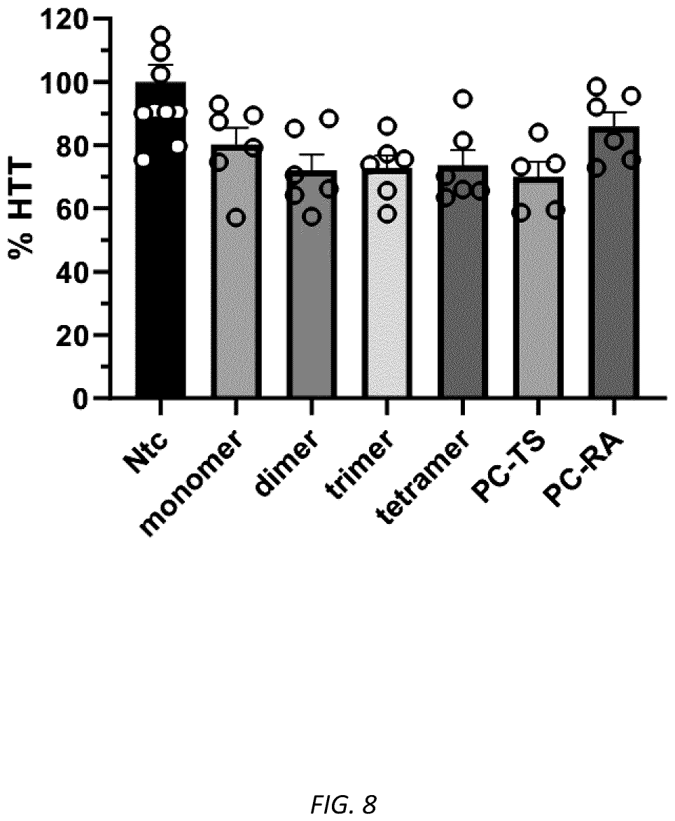

bDNA assay was used to quantify total Htt mRNA levels two weeks after injection with the Htt-siRNA using 0.1 nanomole per injection of stated siRNA modification. 0.1 nanomole resulted in about 20%-30% knockdown at 2 weeks post injection when quantifying Htt mRNA levels as shown in FIG. 8.

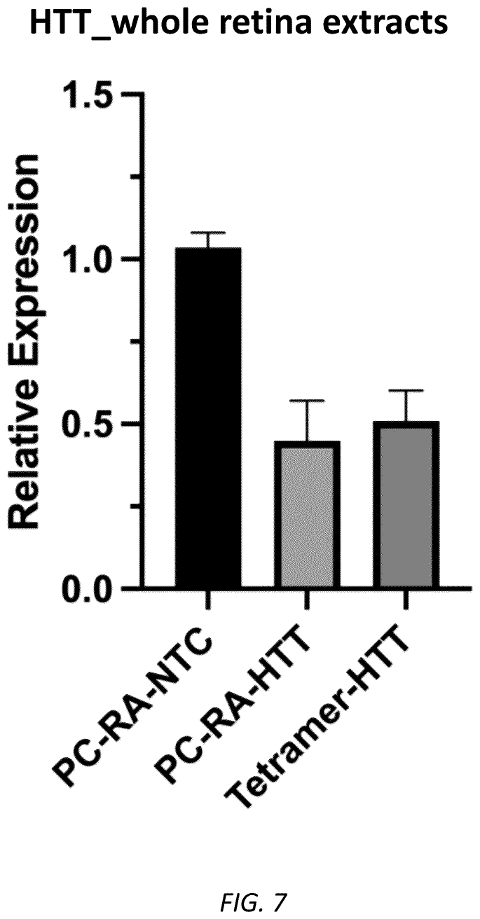

bDNA assay was also used to quantify total Htt mRNA levels 3 days after injection with the Htt-siRNA using 0.3 nanomoles per injection of stated siRNA modification. Note, 0.3 nanomoles results in about 30%-60% knockdown at 3 days post injection when quantifying Htt mRNA levels. Also, PC-RA shows similar percentage knockdown when compared to total protein measurements at 2 weeks post injection (FIG. 7: 60% knockdown), indicating that both quantifications methods are similar for the Htt gene in the retina and that there is a direct correlation between mRNA and protein levels for this gene (each dot represents 1 retina).

### Example 3: Long-Term Assessment of Htt mRNA Knockdown by bDNA Assays

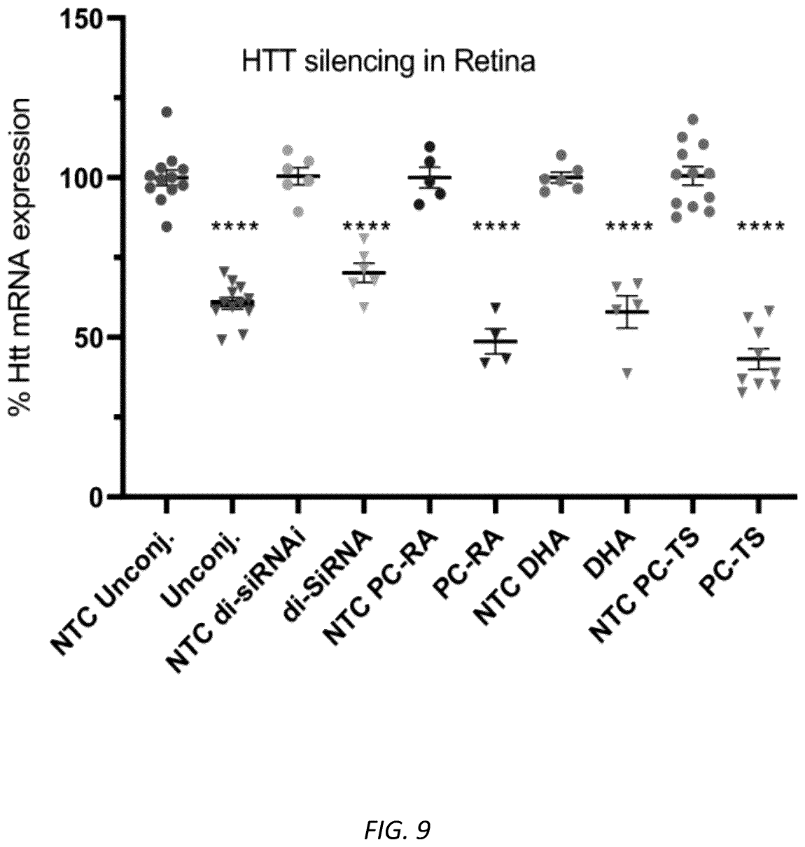

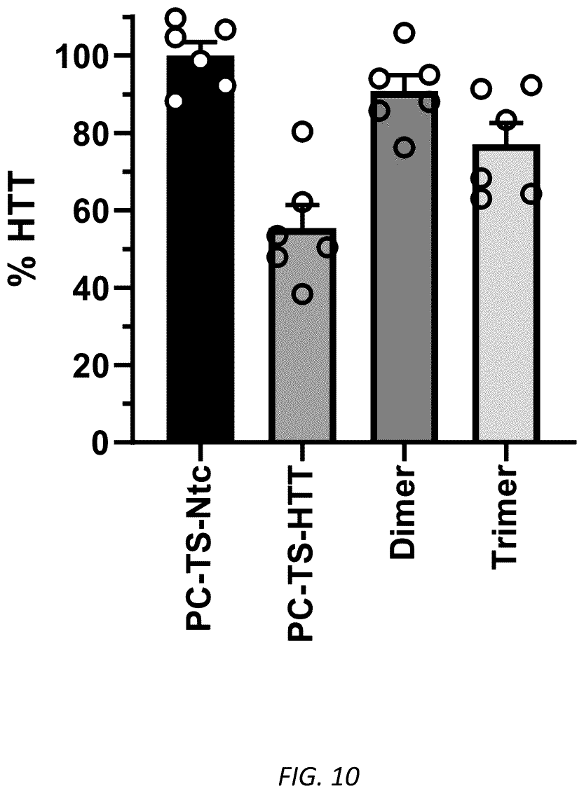

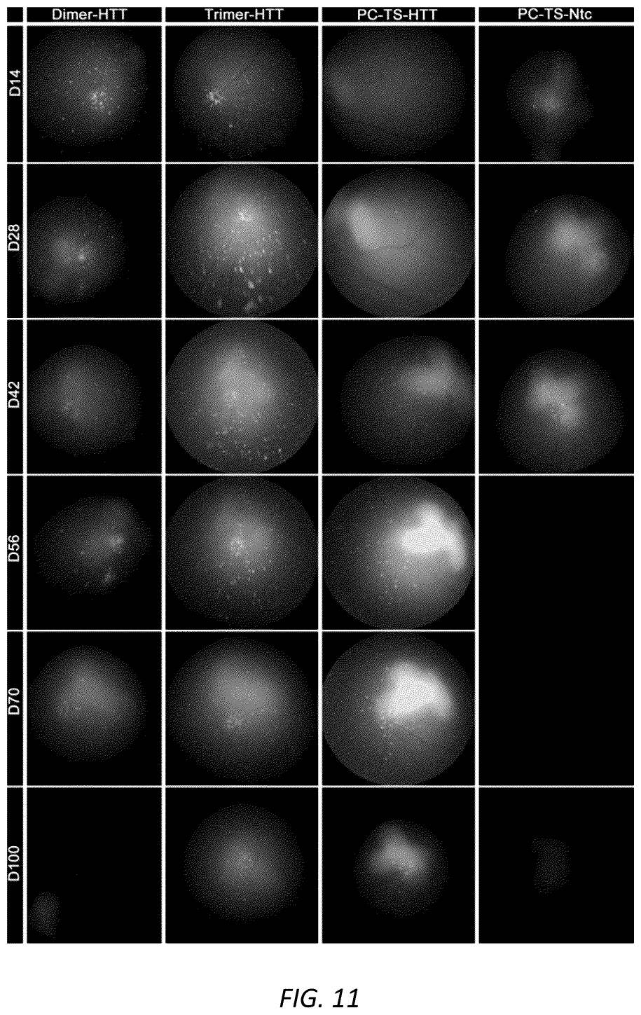

FIG. 10 shows the results of the quantification by bDNA assay performed to quantify total Htt mRNA levels 100 days after injection with the Htt-siRNA using 0.3 nanomoles per injection of stated siRNA modification. Note, compared to FIG. 9 the knockdown effect changed only by ˜10% over a time window of ˜100 days (60% to 50% knockdown), indicating that the knockdown is very stable (each dot represents 1 retina in FIG. 10). FIG. 11 shows representative fundus images over time of eyes injected with the Cy3 labeled siRNAs with modifications as indicated on the figure. Exposure of fluorescence signal is the same for all 4 siRNA at any given time point, but not over time. FIG. 11 complements FIG. 10 showing the fundus images of the mice used in FIG. 10. All mice were injected with 0.3 nanomoles of siRNA intravitreally.

### Example 4: Dose Escalation Study of HTT Knockdown in Mouse with Tetramer Configuration

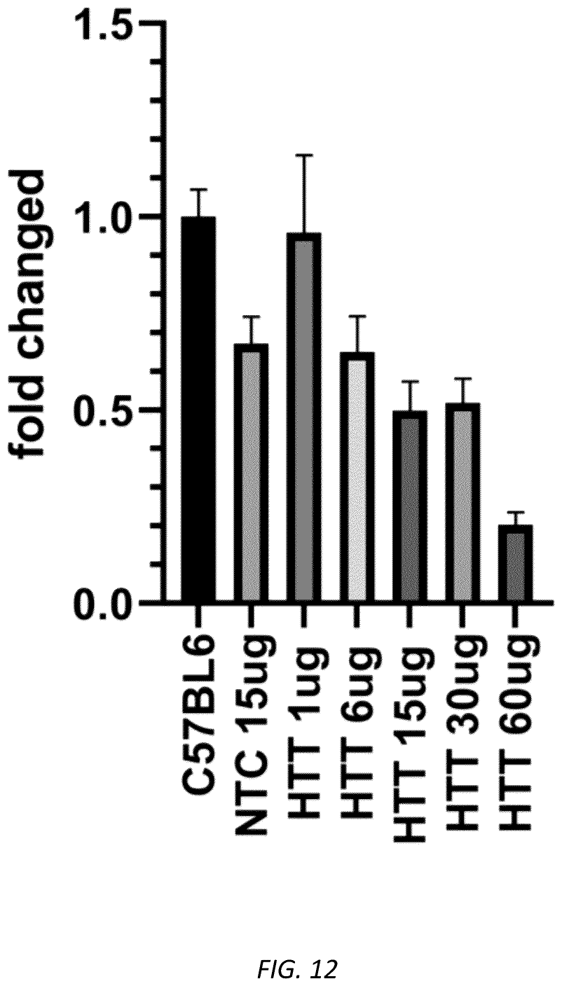

FIG. 12 shows the results of the dose escalation study of for HTT-knockdown in retina. Mice were injected with amounts indicated in the FIG. 1-60 of Cy3 labeled Tetramer with Htt-siRNA) in a total volume of 2 microliter. Five mice were injected per amount of siRNA. Tissue was harvested at 2 weeks post-injection to perform quantification by western blotting of remaining HTT protein in retina. Injections with 15-30 microgram correspond roughly to the same knockdown seen with ˜0.3 nanomoles in previous experiments.

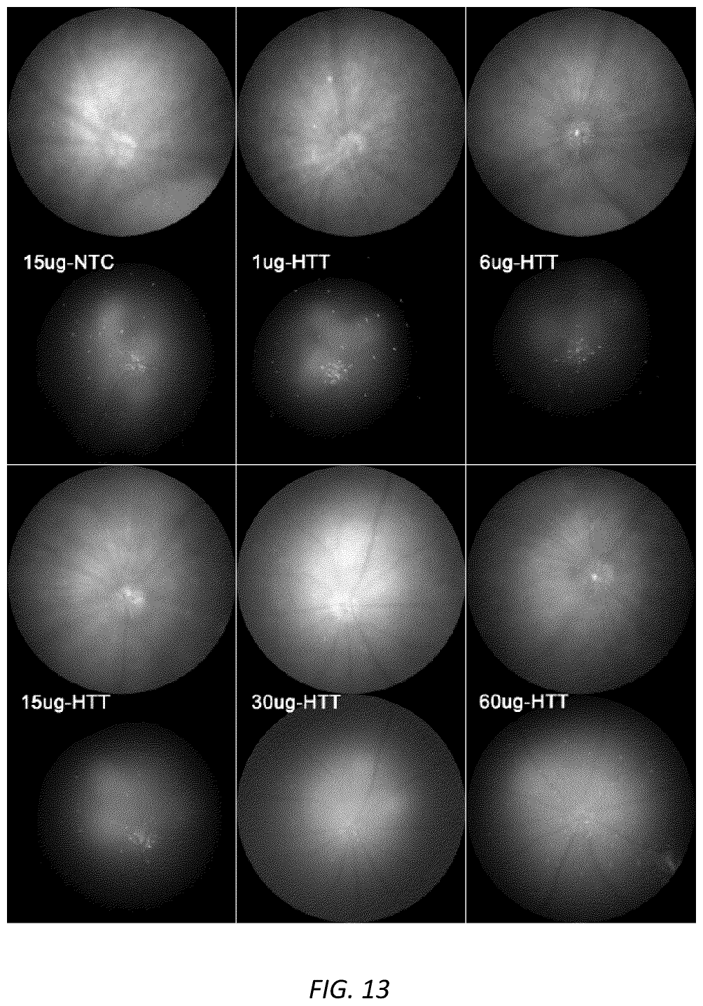

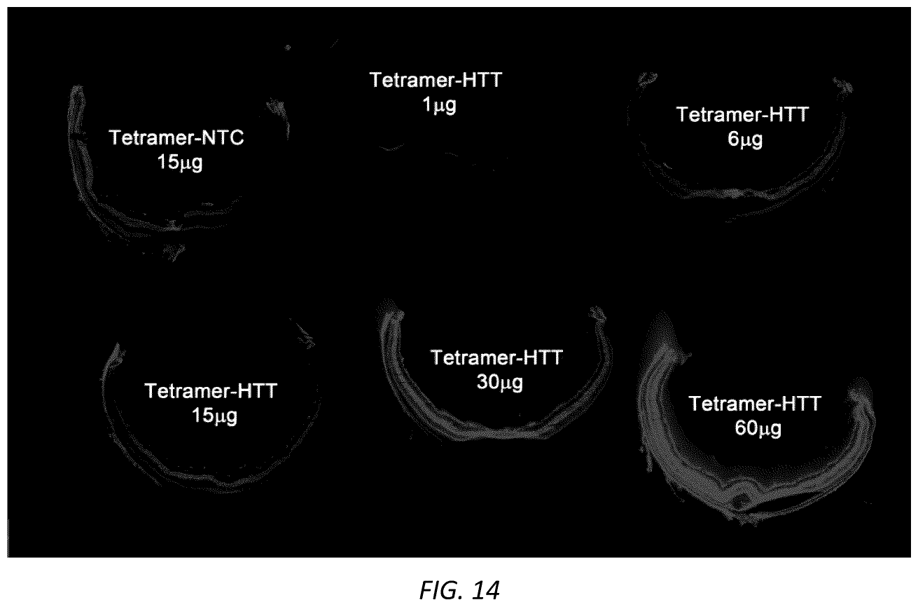

FIG. 14 displays retinal cross sections of eyes from the dose escalation study of the HTT knockdown in mouse with the Tetramer configuration whose results are shown in FIGS. 12 and 13. Images show Cy3 distribution across entire retinal section, indicating that the siRNA is taken up uniformly across the entire eye.

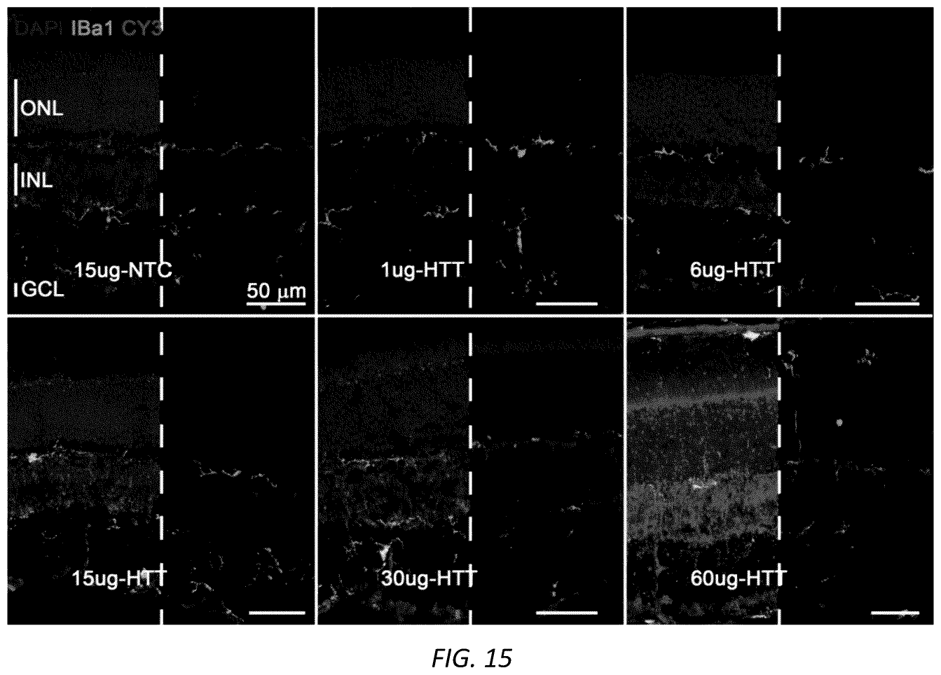

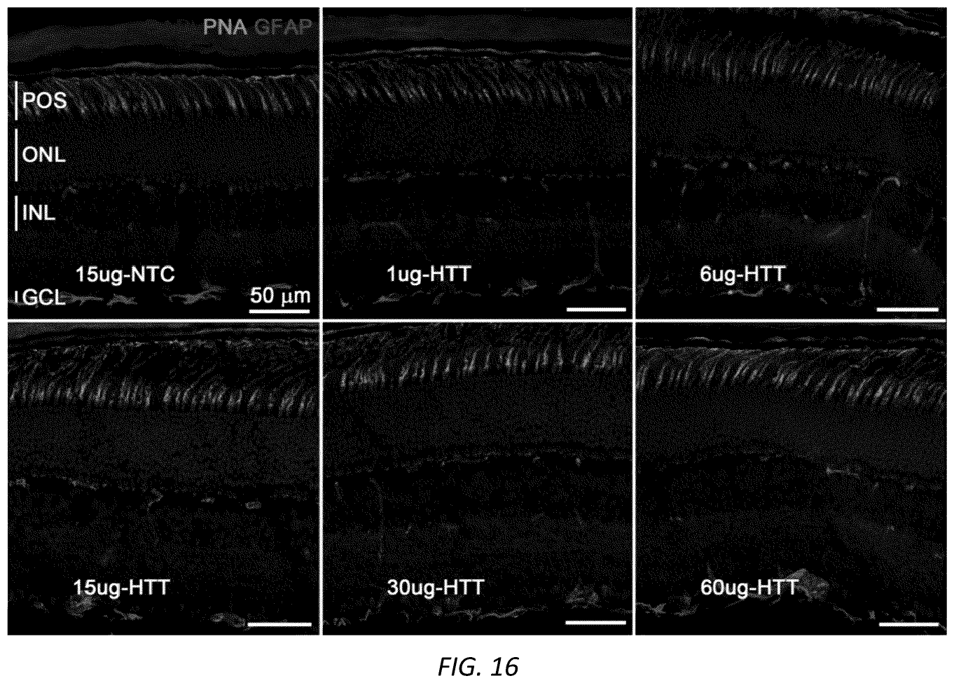

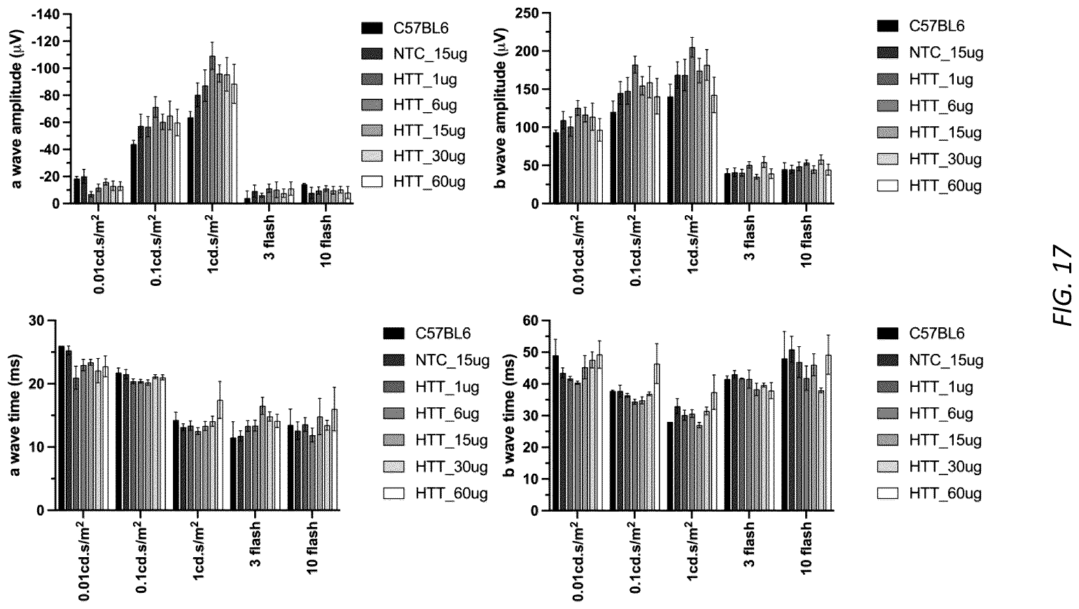

To determine toxicity, antibody staining was performed on retinal sections of eyes shown in FIG. 14 to identify Iba1 positive cells as well as changes in GFAP. FIG. 15 displays these retinal cross sections of eyes stained with Iba1 (green) to identify Iba1 positive cells that migrate to the outer nuclear layer (ONL) where photoreceptors reside. Iba1 positive cells in the ONL are seen at 60 microgram and occasionally at 30 microgram per injection indicating an inflammatory response at 60 microgram and a mild response at 30 microgram. Half of each panel (dotted line on FIG. 15) shows only the Iba1 signal to better visualize the signal Blue shows nuclear DAPI. FIG. 16 displays retinal cross sections of eyes from of dose escalation study shown in FIG. 14 stained with GFAP (red) to identify reactive gliosis in Müller glia cells. While a slight increase in GFAP expression is seen at the ganglion cell layer (GCL) level where astrocytes reside, the expression does not extent upwards into Müller glia cells. Expression of GFAP in astrocytes is normal. The expression is increased with 60 microgram, which is consistent with the results seen with Iba1. However, the absence of reactive gliosis indicates that there are no severe retinal degenerative events induced by the siRNA. siRNA is not shown as these are sections from the same eyes as shown in FIG. 15. In FIG. 16, the color blue indicates nuclear DAPI and green marks cone photoreceptor segment with peanut agglutinin lectin (PNA). FIG. 17 presents the measurements of photoreceptor and retinal function by electroretinography under scotopic (0.01 cd·s/m2-1 cd·s/m2) and photopic conditions (3 & 10 flashes). A-waves and b-waves recording show normal photoreceptor and inner retinal function, respectively for all amounts injected. There is no statistically significant difference between the recordings (n=5 mice per amount of siRNA) as seen in the upper two graphs. Implicit time of a- and b-waves (lower two graphs) are also not significantly different between the different groups of mice injected.

### Example 5: siRNA in Eye of a Large Animal Model: The Swine (All Data Shown Below is Generated With the Tetramer-Htt-siRNA-Cy3 and its NTC in Swine)

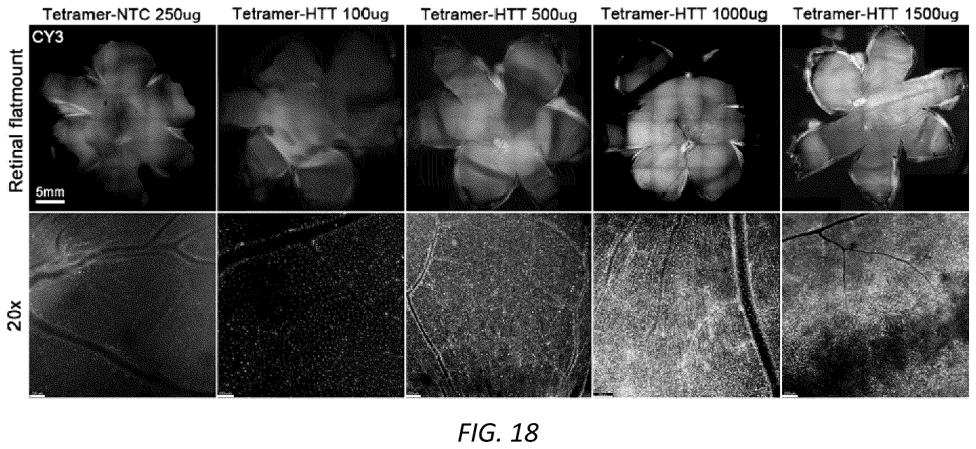

For translational purposes of the siRNA technology, the suitability of the technique in a large animal model was tested to determine distribution, knockdown efficiency and toxicity. To that end, the pig model was chosen as pigs have eyes similar in size to humans (35 kg pigs were used). The only difference is the absence of a fovea. As an initial test run, 3 pigs were injected with 5 different amounts of siRNA of the same chemical configuration, keeping the injection volume constant at 100 microliter. The following data represent a summary of the injections in pigs with the siRNA against HTT in the Tetramer configuration. All siRNA molecules were also labeled with Cy3. Pigs were euthanized 10 days after the intravitreal injection. FIG. 18 shows the fluorescence intensity of Tetramer-Htt-Cy3 after intravitreal delivery in pig eye. Delivery of amount of siRNA is shown on top of each panel in FIG. 18 (100-1500 microgram of Tetramer). Fluorescence intensity is well distributed across the entire eye. Top row in FIG. 18 shows the Cy3 fluorescence of unfixed tissue right after opening the eye, meanwhile the bottom panel is a higher magnification of a region from the top panel.

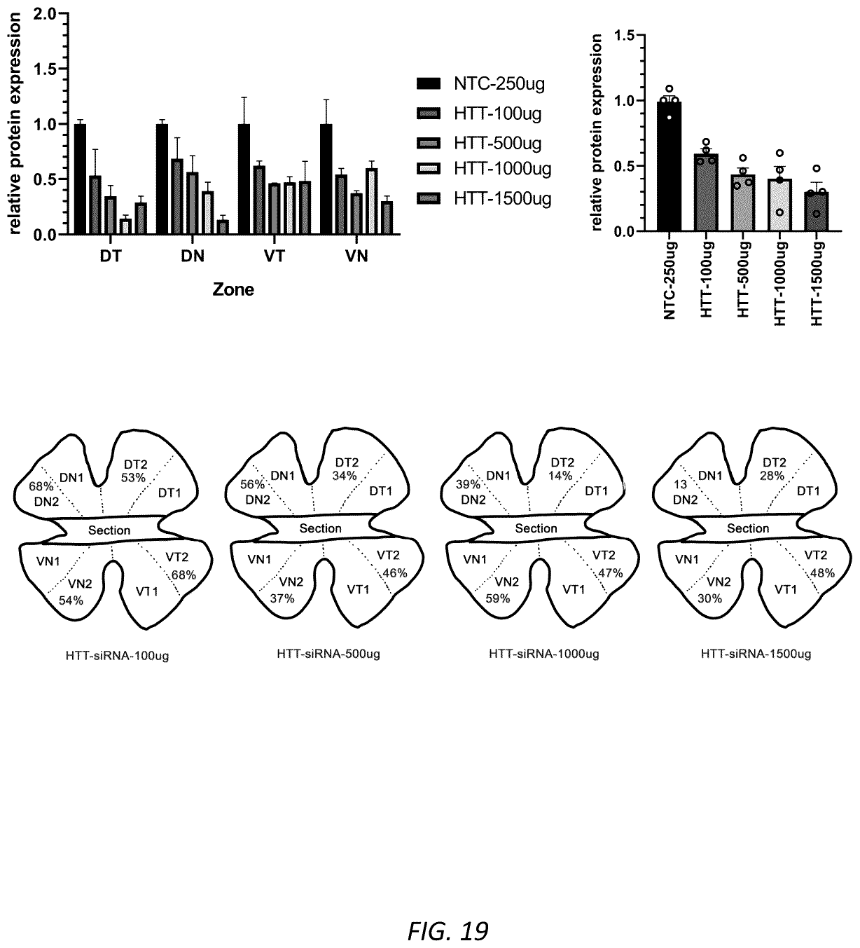

Knockdown of Huntington protein in Swine was measured from pigs' eyes shown in FIG. 18 by western blot analysis. Knockdown was compared to Huntington protein levels in the NTC that was injected with 250ug of the Tetramer-siRNA-Cy3. In FIG. 19, Top figure shows knockdown in bar graphs seen in the four major retinal quadrants (DT: Dorsal-Temporal; DN: Dorsal-Nasal; VT: Temporal-Nasal; VN: Ventral-Nasal) with the error bars being generated by technical replicates. The knockdown efficiency in each quadrant is dependent on the positioning of the needle and the angle of insertion. Needles were generally inserted from the temporal side and pointed towards the center of the eye. Middle panel in FIG. 19 shows knockdown on a flat mount cartoon with corresponding values of the regional knockdown shown in the bar graph. Bottom panel in FIG. 19 shows the average knockdown of Huntington protein across the entire retina calculated by averaging the knockdown seen in each quadrant per retina with the error bars being generated by averaging the 4 data points for each quadrant per retina. Data shown in FIG. 19's bottom panel represents one biological sample for each amount of siRNA delivered.

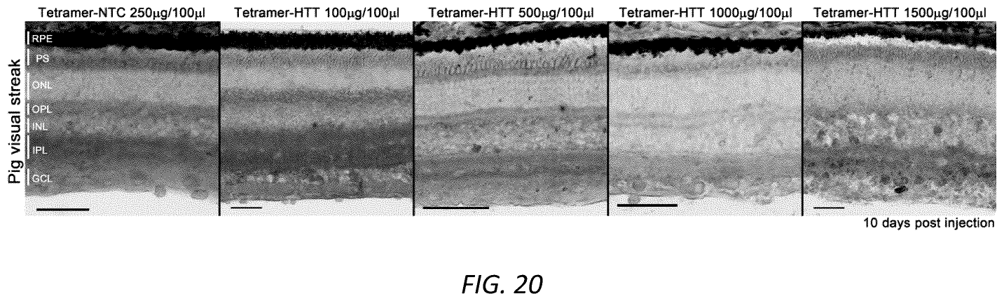

FIG. 20 displays antibody staining for Huntington protein on section of eyes injected with different amounts as shown in FIG. 19. The area of the sections of eyes is shown in the middle panel of FIG. 19. Huntington knockdown is seen across all retinal layers and in particular in the Inner and Outer Plexiform Layers (IPL, OPL), and where the photoreceptor segment (PS) is located.

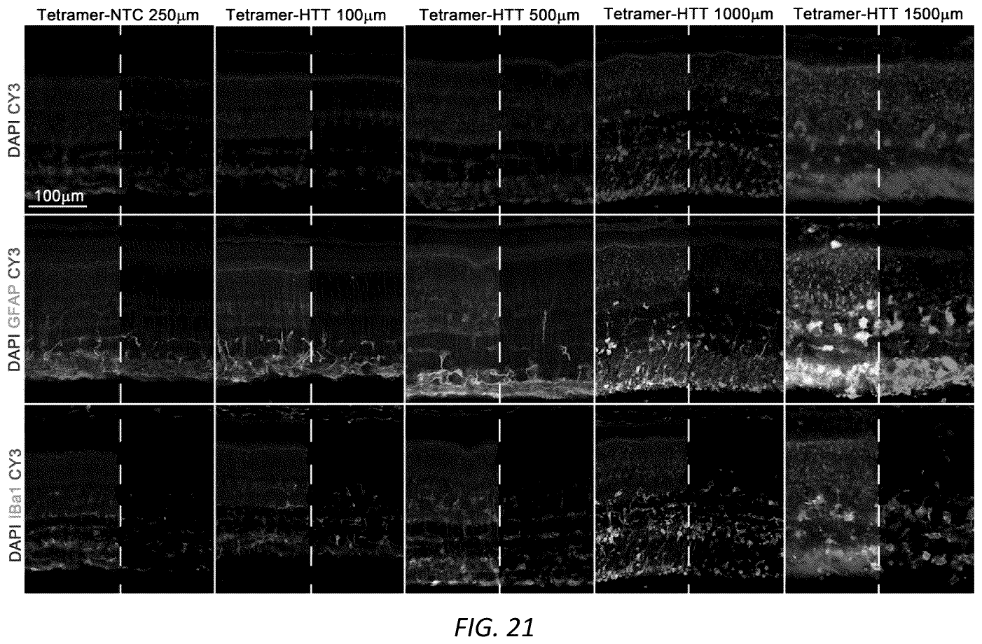

FIG. 21 displays antibody staining for GFAP (glial fibrillary acidic protein) and Iba1 (ionized calcium binding adaptor protein 1) (as shown in mouse on FIGS. 15 and 16) expression on retinal section of eyes injected with different amount as shown in FIGS. 18 and 19 to determine dose dependent toxicity. GFAP and Iba1 are both shown in green in FIG. 21 as indicated to the left of each row. Red staining in FIG. 21 shows siRNA distribution across the retinal section while nuclei are marked with nuclear DAPI. There is a clear dose dependent increase of GFAP and Iba1 expression. Up to 500 ug of siRNA expression of IBA1 and GFAP is rarely seen to progress into the outer nuclear layer (ONL) where photoreceptors reside. At 1000 ug and 1500 ug there is a visible increase in GFAP and Iba1 expression in the ONL. Additionally, a lot of the siRNA appears be taken up by Iba1 positive cells, which reflect likely macrophages that take up excess extracellular material. Half of each panel in FIG. 21 shows only the signal of interest (siRNA, GFAP or Iba1) to better visualize the signal.

Summary of Pig data: The Tetramer-Htt-siRNA distributes well across the entire retina after one intravitreal delivery in a large eye as the swine. This is particularly important as pig eyes are of similar size as human eyes. Besides lacking a fovea, the pig eye is the closest animal model to the human eye. For distribution studies it is more relevant due to the similar size when compared to most lab NHPs. The dose response in FIG. 19 and the toxicity in FIG. 21 show that for this particular compound doses in the range of 100-500 ug might be used for further studies. This should result to an approximate 50% knockdown of HTT which is similar to what is seen in mouse with the tetramer. Toxicity may be reduced by the removal of the Cy3 molecule, which was still attached in the current study.

### Example 6: Development of an siRNA Against S6K1 (RPS6KB1: Ribosomal Protein S6 Kinase B1)

An initial bioinformatics screen was performed to identify potential siRNA sequences for S6K1. A list of sequences that were identified in the initial bioinformatics screen are shown below in Table 1 and Table 2.

TABLE 1
S6K1 45-nucleotide gene region target sites

| Oligo ID | Gene region |
|---|---|
| Rps6kb1_4456 | TTATTTTCTTTAAAATCAGCTATTACAGGATATTT |
| | TTTTATTTTC |
| Rps6kb1_459 | AATACTGGGAAGATATTTGCCATGAAGGTGCTTAA |
| | AAAGGCAATG (SEQ ID NO: 1) |
| Rps6kb1_291 | GTTGGACCATATGAACTTGGCATGGAACATTGTGA |
| | GAAATTTGAA (SEQ ID NO: 2) |
| Rps6kb1_4893 | TTCCTAGGTTACAAGGGCTAGATCTAAGATTATTC |
| | TCATGAGAAA |
| Rps6kb1_431 | TTTTCAAGTACGAAAAGTAACAGGAGCAAATACTG |
| | GGAAGATATT |
| Rps6kb1_4561 | TAAGGTTTGTAGTGTTACAGAATAACTAAACTGGG |
| | ATTTATAAAC |
| Rps6kb1_2104 | TTGCCTGTAATACTTGCAACTAAGGACAAATTAGC |
| | ATGCAAGCTT (SEQ ID NO: 3) |
| Rps6kb1_1217 | TCCCTTTAAGCCTCTGTTGCAATCTGAAGAGGATG |
| | TGAGTCAGTT |
| Rps6kb1_2200 | ATTGATGTTTTACGTGCAAACAACCTGAATCTTTT |
| | TTTTATATAA (SEQ ID NO: 4) |
| Rps6kb1_1364 | ATCTGTACTTGAAAGTGTGAAAGAAAAGTTTTCAT |
| | TTGAACCAAA (SEQ ID NO: 5) |
| Rps6kb1_3733 | GACTGTAGCAGCATTTTGAGAACTTCATAATTGTA |
| | GCAGTAAATT |
| Rps6kb1_4801 | ATAAACAAAATGTATCTTAGCATTAATATCTTGAG |
| | CCTTGAACAT |
| Rps6kb1_3133 | GGGTTTAATATCTGCAGAGCTTTATAAAATATACT |
| | GCAGTGCATA |
| Rps6kb1_4476 | TATTACAGGATATTTTTTTATTTTCTACATTCTGT |
| | TTTTTAATTA |
| Rps6kb1_4378 | TTCTAAGAGAGGTGTTCATGCTTGTACCAGGTAAG |
| | TGAATAAAAA |
| Rps6kb1_2809 | TACTGTTATGCTGTTTACCTTCCTTAACAATTTTC |
| | TTTTTTGAGA |
| Rps6kb1_2539 | CGTAACTGCAAGCCTTGGGACAGGCAGAAGTTGTA |
| | TGATCTACAT |
| Rps6kb1_4323 | TTTATTTGTAAAAACTGAAGCATAATTTAAAGTGT |
| | ATATCAATAC |
| Rps6kb1_2049 | CCGTTGAAATCTGATGATGTCAAATAAGGGTTATC |
| | CTAATAGGCA (SEQ ID NO: 6) |
| Rps6kb1_3965 | AATTCCAATAAAACTTTTACACTAAATGATTCCTC |
| | CTTAGCCCTA |
| Rps6kb1_1893 | AAACCCACAAAAAACTCAAGCAAAATAGTATTGTG |
| | GAATCCACAG |

For the above recited 45-nucleotide gene regions, the sequences correspond to the DNA gene sequence, however the mRNA encoded by the S6K1 gene will have the same sequences with T nucleotides replaced with U nucleotides. Accordingly, and by way of example, an siRNA with an antisense strand that targets SEQ ID NO: 1 will target the mRNA sequence that corresponds to the gene region of SEQ ID NO: 1.

TABLE 2
S6K1 20-nucleotide target sites

| Oligo ID | Sequence |
|---|---|
| Rps6kb1_4456 | UCAGCUAUUACAGGAUAUUU |
| Rps6kb1_459 | UUUGCCAUGAAGGUGCUUAA |
| | (SEQ ID NO: 7) |
| Rps6kb1_291 | CUUGGCAUGGAACAUUGUGA |
| | (SEQ ID NO: 8) |
| Rps6kb1_4893 | GGCUAGAUCUAAGAUUAUUC |
| Rps6kb1_431 | AGUAACAGGAGCAAAUACUG |
| Rps6kb1_4561 | UACAGAAUAACUAAACUGGG |
| Rps6kb1_2104 | GCAACUAAGGACAAAUUAGC |
| | (SEQ ID NO: 9) |
| Rps6kb1_1217 | GUUGCAAUCUGAAGAGGAUG |
| Rps6kb1_2200 | GCAAACAACCUGAAUCUUUU |
| | (SEQ ID NO: 10) |
| Rps6kb1_1364 | UGUGAAAGAAAAGUUUUCAU |
| | (SEQ ID NO: 11) |
| Rps6kb1_3733 | UUGAGAACUUCAUAAUUGUA |
| Rps6kb1_4801 | CUUAGCAUUAAUAUCUUGAG |
| Rps6kb1_3133 | AGAGCUUUAUAAAAUAUACU |
| Rps6kb1_4476 | UUUUAUUUUCUACAUUCUGU |
| Rps6kb1_4378 | UCAUGCUUGUACCAGGUAAG |
| Rps6kb1_2809 | UACCUUCCUUAACAAUUUUC |
| Rps6kb1_2539 | UGGGACAGGCAGAAGUUGUA |
| Rps6kb1_4323 | UGAAGCAUAAUUUAAAGUGU |
| Rps6kb1_2049 | GAUGUCAAAUAAGGGUUAUC |
| | (SEQ ID NO: 12) |
| Rps6kb1_3965 | UUUACACUAAAUGAUUCCUC |
| Rps6kb1_1893 | UCAAGCAAAAUAGUAUUGUG |

TABLE 3
S6K1 sense strands

| Duplex # | Oligo ID | Sense Sequence |
|---|---|---|
| 1 | Rps6kb1_4456 | (mU)#(mA)#(mU)(mU)(fA)(fC)(fA)(mG)(fG)(mA)(mU)(mA)(mU) |
| | | #(mU)#(mA)-TegChol |
| 2 | Rps6kb1_459 | (mC)#(mA)#(mU)(mG)(fA)(fA)(fG)(mG)(fU)(mG)(mC)(mU)(mU) |
| | | #(mA)#(mA)-TegChol |
| 3 | Rps6kb1_291 | (mC)#(mA)#(mU)(mG)(fG)(fA)(fA)(mC)(fA)(mU)(mU)(mG)(mU) |
| | | #(mG)#(mA)-TegChol |
| 4 | Rps6kb1_4893 | (mG)#(mA)#(mU)(mC)(fU)(fA)(fA)(mG)(fA)(mU)(mU)(mA)(mU) |
| | | #(mU)#(mA)-TegChol |
| 5 | Rps6kb1_431 | (mC)#(mA)#(mG)(mG)(fA)(fG)(fC)(mA)(fA)(mA)(mU)(mA)(mC) |
| | | #(mU)#(mA)-TegChol |
| 6 | Rps6kb1_4561 | (mA)#(mA)#(mU)(mA)(fA)(fC)(fU)(mA)(fA)(mA)(mC)(mU)(mG) |
| | | #(mG)#(mA)-TegChol |
| 7 | Rps6kb1_2104 | (mU)#(mA)#(mA)(mG)(fG)(fA)(fC)(mA)(fA)(mA)(mU)(mU)(mA) |
| | | #(mG)#(mA)-TegChol |
| 8 | Rps6kb1_1217 | (mA)#(mA)#(mU)(mC)(fU)(fG)(fA)(mA)(fG)(mA)(mG)(mG)(mA) |
| | | #(mU)#(mA)-TegChol |
| 9 | Rps6kb1_2200 | (mC)#(mA)#(mA)(mC)(fC)(fU)(fG)(mA)(fA)(mU)(mC)(mU)(mU) |
| | | #(mU)#(mA)-TegChol |
| 10 | Rps6kb1_1364 | (mA)#(mA)#(mG)(mA)(fA)(fA)(fA)(mG)(fU)(mU)(mU)(mU)(mC) |
| | | #(mA)#(mA)-TegChol |
| 11 | Rps6kb1_3733 | (mA)#(mA)#(mC)(mU)(fU)(fC)(fA)(mU)(fA)(mA)(mU)(mU)(mG) |
| | | #(mU)#(mA)-TegChol |
| 12 | Rps6kb1_4801 | (mC)#(mA)#(mU)(mU)(fA)(fA)(fU)(mA)(fU)(mC)(mU)(mU)(mG) |
| | | #(mA)#(mA)-TegChol |
| 13 | Rps6kb1_3133 | (mU)#(mU)#(mU)(mA)(fU)(fA)(fA)(mA)(fA)(mU)(mA)(mU)(mA) |
| | | #(mC)#(mA)-TegChol |
| 14 | Rps6kb1_4476 | (mU)#(mU)#(mU)(mU)(fC)(fU)(fA)(mC)(fA)(mU)(mU)(mC)(mU) |
| | | #(mG)#(mA)-TegChol |
| 15 | Rps6kb1_4378 | (mC)#(mU)#(mU)(mG)(fU)(fA)(fC)(mC)(fA)(mG)(mG)(mU)(mA) |
| | | #(mA)#(mA)-TegChol |
| 16 | Rps6kb1_2809 | (mU)#(mC)#(mC)(mU)(fU)(fA)(fA)(mC)(fA)(mA)(mU)(mU)(mU) |
| | | #(mU)#(mA)-TegChol |
| 17 | Rps6kb1_2539 | (mC)#(mA)#(mG)(mG)(fC)(fA)(fG)(mA)(fA)(mG)(mU)(mU)(mG) |
| | | #(mU)#(mA)-TegChol |
| 18 | Rps6kb1_4323 | (mC)#(mA)#(mU)(mA)(fA)(fU)(fU)(mU)(fA)(mA)(mA)(mG)(mU) |
| | | #(mG)#(mA)-TegChol |
| 19 | Rps6kb1_2049 | (mC)#(mA)#(mA)(mA)(fU)(fA)(fA)(mG)(fG)(mG)(mU)(mU)(mA) |
| | | #(mU)#(mA)-TegChol |
| 20 | Rps6kb1_3965 | (mA)#(mC)#(mU)(mA)(fA)(fA)(fU)(mG)(fA)(mU)(mU)(mC)(mC) |
| | | #(mU)#(mA)-TegChol |
| 21 | Rps6kb1_1893 | (mC)#(mA)#(mA)(mA)(fA)(fU)(fA)(mG)(fU)(mA)(mU)(mU)(mG) |
| | | #(mU)#(mA)-TegChol |

TABLE 4
S6K1 sense strands

| Duplex # | Oligo ID | Antisense Sequence |
|---|---|---|
| 1 | Rps6kb14456 | P(mU)#(fA)#(mA)(mU)(mA)(fU)(mC)(mC)(mU)(mG)(mU)(mA) |
| | | (mA)#(fU)#(mA)#(fG)#(mC)#(mU)#(mG)#(fA) |
| 2 | Rps6kb1459 | P(mU)#(fU)#(mA)(mA)(mG)(fC)(mA)(mC)(mC)(mU)(mU)(mC) |
| | | (mA)#(fU)#(mG)#(fG)#(mC)#(mA)#(mA)#(fA) |
| 3 | Rps6kb1291 | P(mU)#(fC)#(mA)(mC)(mA)(fA)(mU)(mG)(mU)(mU)(mC)(mC) |
| | | (mA)#(fU)#(mG)#(fC)#(mC)#(mA)#(mA)#(fG) |
| 4 | Rps6kb14893 | P(mU)#(fA)#(mA)(mU)(mA)(fA)(mU)(mC)(mU)(mU)(mA)(mG) |
| | | (mA)#(fU)#(mC)#(fU)#(mA)#(mG)#(mC)#(fC) |
| 5 | Rps6kb1431 | P(mU)#(fA)#(mG)(mU)(mA)(fU)(mU)(mU)(mG)(mC)(mU)(mC) |
| | | (mC)#(fU)#(mG)#(fU)#(mU)#(mA)#(mC)#(fU) |
| 6 | Rps6kb14561 | P(mU)#(fC)#(mC)(mA)(mG)(fU)(mU)(mU)(mA)(mG)(mU)(mU) |
| | | (mA)#(fU)#(mU)#(fC)#(mU)#(mG)#(mU)#(fA) |
| 7 | Rps6kb12104 | P(mU)#(fC)#(mU)(mA)(mA)(fU)(mU)(mU)(mG)(mU)(mC)(mC) |
| | | (mU)#(fU)#(mA)#(fG)#(mU)#(mU)#(mG)#(fC) |
| 8 | Rps6kb11217 | P(mU)#(fA)#(mU)(mC)(mC)(fU)(mC)(mU)(mU)(mC)(mA)(mG) |
| | | (mA)#(fU)#(mU)#(fG)#(mC)#(mA)#(mA)#(fC) |
| 9 | Rps6kb12200 | P(mU)#(fA)#(mA)(mA)(mG)(fA)(mU)(mU)(mC)(mA)(mG)(mG) |
| | | (mU)#(fU)#(mG)#(fU)#(mU)#(mU)#(mG)#(fC) |
| 10 | Rps6kb11364 | P(mU)#(fU)#(mG)(mA)(mA)(fA)(mA)(mC)(mU)(mU)(mU)(mU) |
| | | (mC)#(fU)#(mU)#(fU)#(mC)#(mA)#(mC)#(fA) |
| 11 | Rps6kb13733 | P(mU)#(fA)#(mC)(mA)(mA)(fU)(mU)(mA)(mU)(mG)(mA)(mA) |
| | | (mG)#(fU)#(mU)#(fC)#(mU)#(mC)#(mA)#(fA) |
| 12 | Rps6kb14801 | P(mU)#(fU)#(mC)(mA)(mA)(fG)(mA)(mU)(mA)(mU)(mU)(mA) |
| | | (mA)#(fU)#(mG)#(fC)#(mU)#(mA)#(mA)#(fG) |
| 13 | Rps6kb13133 | P(mU)#(fG)#(mU)(mA)(mU)(fA)(mU)(mU)(mU)(mU)(mA)(mU) |
| | | (mA)#(fA)#(mA)#(fG)#(mC)#(mU)#(mC)#(fU) |
| 14 | Rps6kb14476 | P(mU)#(fC)#(mA)(mG)(mA)(fA)(mU)(mG)(mU)(mA)(mG)(mA) |
| | | (mA)#(fA)#(mA)#(fU)#(mA)#(mA)#(mA)#(fA) |
| 15 | Rps6kb14378 | P(mU)#(fU)#(mU)(mA)(mC)(fC)(mU)(mG)(mG)(mU)(mA)(mC) |
| | | (mA)#(fA)#(mG)#(fC)#(mA)#(mU)#(mG)#(fA) |
| 16 | Rps6kb12809 | P(mU)#(fA)#(mA)(mA)(mA)(fU)(mU)(mG)(mU)(mU)(mA)(mA) |
| | | (mG)#(fG)#(mA)#(fA)#(mG)#(mG)#(mU)#(fA) |
| 17 | Rps6kb12539 | P(mU)#(fA)#(mC)(mA)(mA)(fC)(mU)(mU)(mC)(mU)(mG)(mC) |
| | | (mC)#(fU)#(mG)#(fU)#(mC)#(mC)#(mC)#(fA) |
| 18 | Rps6kb14323 | P(mU)#(fC)#(mA)(mC)(mU)(fU)(mU)(mA)(mA)(mA)(mU)(mU) |
| | | (mA)#(fU)#(mG)#(fC)#(mU)#(mU)#(mC)#(fA) |
| 19 | Rps6kb12049 | P(mU)#(fA)#(mU)(mA)(mA)(fC)(mC)(mC)(mU)(mU)(mA)(mU) |
| | | (mU)#(fU)#(mG)#(fA)#(mC)#(mA)#(mU)#(fC) |
| 20 | Rps6kb13965 | P(mU)#(fA)#(mG)(mG)(mA)(fA)(mU)(mC)(mA)(mU)(mU)(mU) |
| | | (mA)#(fG)#(mU)#(fG)#(mU)#(mA)#(mA)#(fA) |
| 21 | Rps6kb11893 | P(mU)#(fA)#(mC)(mA)(mA)(fU)(mA)(mC)(mU)(mA)(mU)(mU) |
| | | (mU)#(fU)#(mG)#(fC)#(mU)#(mU)#(mG)#(fA) |

For the sense and antisense sequences of Table 3 and 3, “m” corresponds to a 2′-O-methyl modified nucleotide, “f” corresponds to a 2′-fluoro modified nucleotide, “#” corresponds to a phosphorothioate internucleotide linkage, “P” corresponds to a 5′ phosphate, and “TegChol” corresponds to a tri- or tetra-ethylene glycol linked cholesterol moiety.

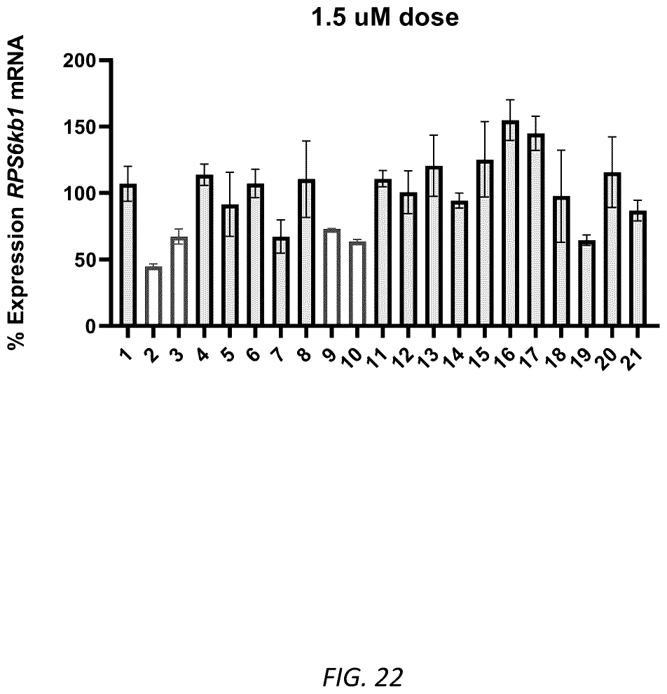

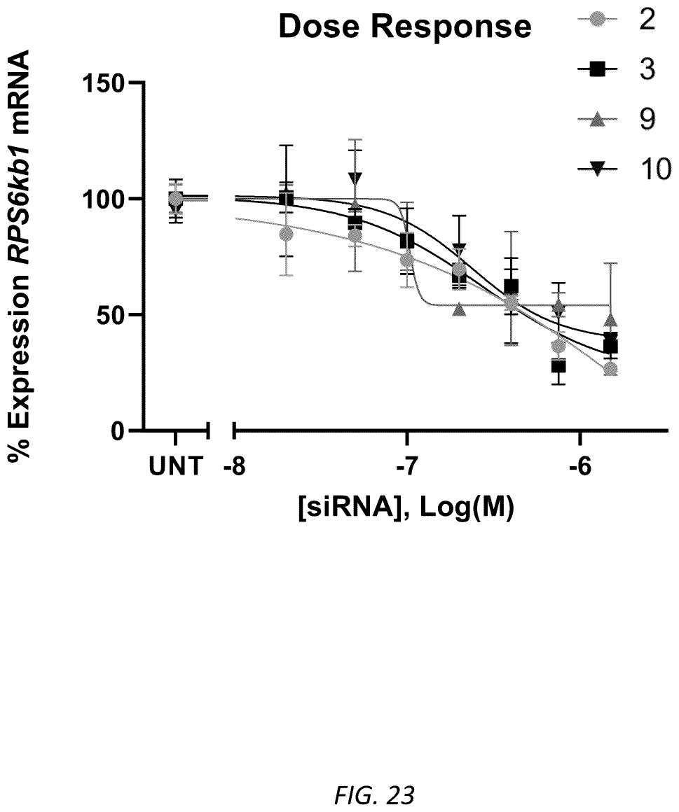

FIG. 22 shows the initial knockdown efficiency in vitro of the duplexes formed from the sense and antisense strands shown in Table 3 and 4. Candidates with the best knockdown results were duplexes 2, 3, 7, 9, 10, and 19. FIG. 23 shows dose response curves for the 4 sequences highlighted in red in FIG. 22. Duplex 2 (Rps6k1b_459) showed the most consistent response and was therefore selected for further studies in vivo.

The primary objective of the siRNA for S6K1 is to knockdown S6K1 in photoreceptors for the treatment of AMD. Based on the data generated by the different HTT-siRNA conjugates, an siRNA was developed initially in the tetramer configuration without any Cy3 label to reduce toxicity. In vivo data from mouse and NHP was then generated, based on the tetramer configuration for duplex 2 (Rps6k1b_459).

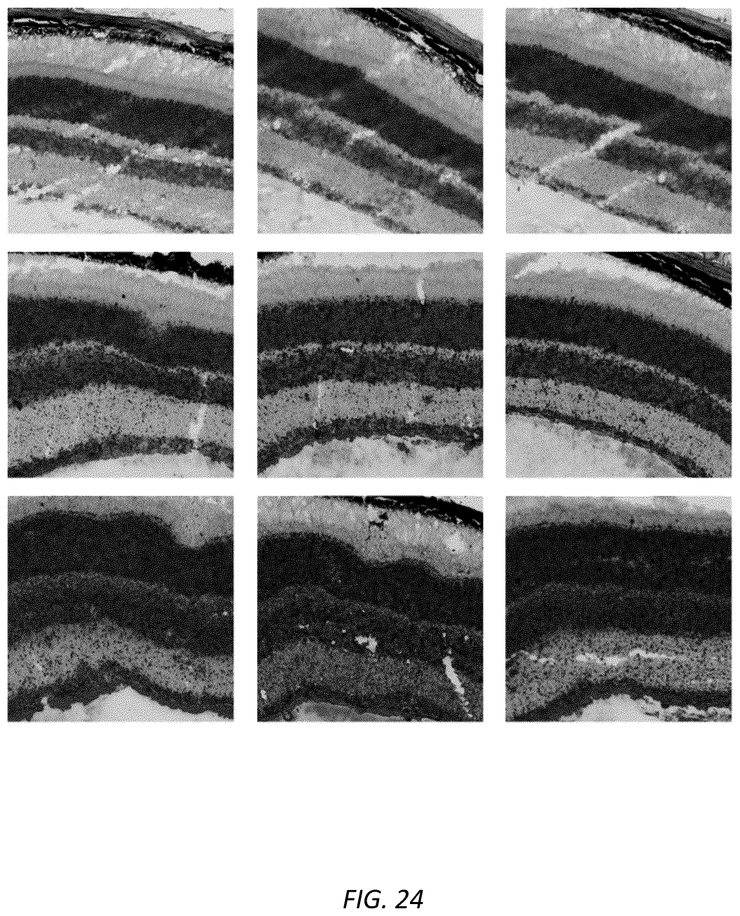

FIG. 24 shows an RNA-Scope in situ hybridization on retinal cross-sections of mice to detect the siRNA-Tetramer against S6K1. The top row in FIG. 24 shows sections from 3 mice injected with the NTC for S6K1 in the tetramer configuration; the middle row shows sections from 3 mice injected with the 3 μg/eye with the siRNA against S6K1 in the tetramer configuration; and the last row shows sections from 3 mice injected with the 6 ug/eye with the siRNA against S6K1 in the tetramer configuration. siRNA was delivered intravitreally and animals were euthanized 2 weeks post injection.

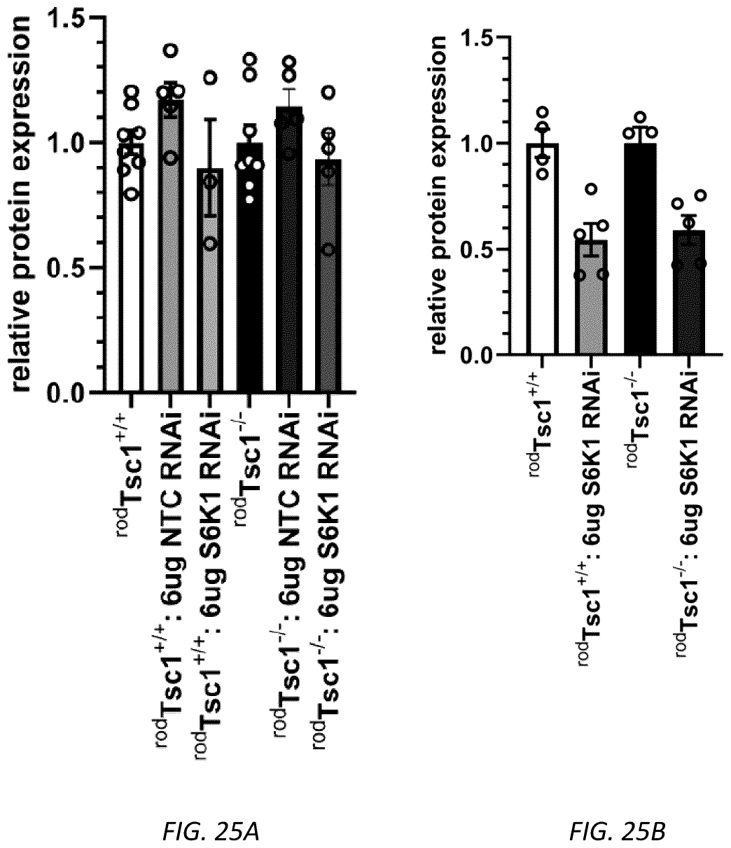

FIG. 25A and FIG. 25B display knockdown of S6K1 in mouse after intravitreal injection of 611 g of siRNA in the Tetramer configuration. Both graphs in FIG. 25 use rodTSC1−/− mice that have been shown to develop age-related macular degeneration like pathologies. The rodTSC1+/+ mice serve are Cre-negative littermate controls that do not develop pathologies. FIG. 25A shows S6K1 protein levels as detected by western blot 2-weeks post injection. A small tendency of reduced S6K1 protein is seen when compared to uninjected littermates or NTC mice. FIG. 25B shows similar data as first graph at 2 months post-injection. A strong knockdown is seen (40-45%) with 6 μg of siRNA. Each dot in the graphs in FIG. 25 represent one biological sample (retina) from one animal.

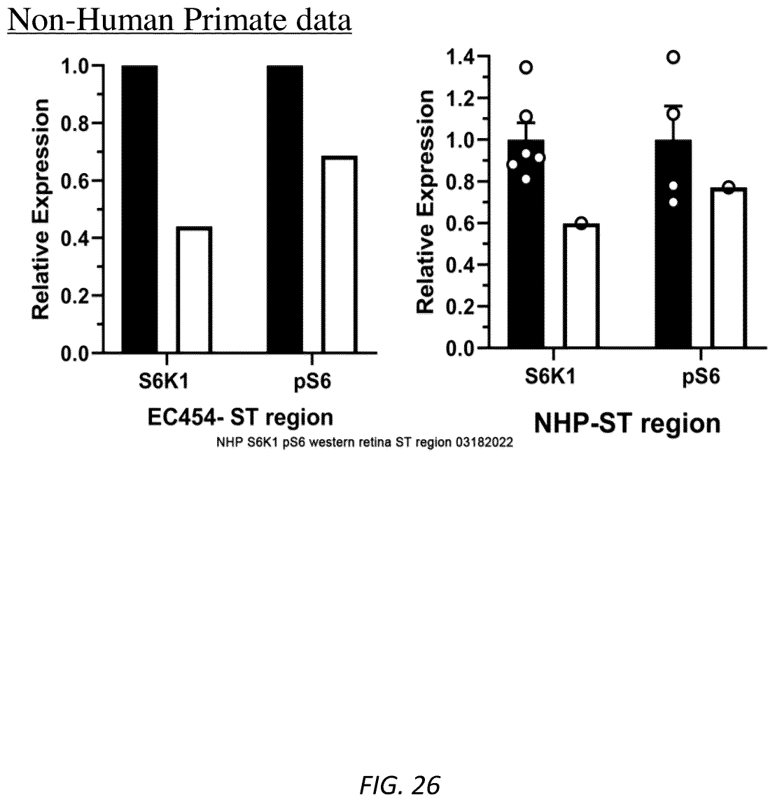

FIG. 26 shows the knockdown of S6K1 protein in non-human primate (NHP). Western blot data with retinal protein extracts form the superior-temporal (ST) regions (AKA: dorsal-temporal) of one NHP injected intravitreally with 225 μg of S6K1-tetramer (in 75 uL) and 6 naïve NHP retinas from the same region. First set of bar graphs shows a comparison between the uninjected contralateral eye and the S6K1 siRNA injected one to allow for a direct intra-animal comparison between both eyes. The second bar graph shows a comparison between the 6 naïve NHPs and the S6K1 siRNA injected one. The knockdown efficiency of S6K1 appears in both cases around 50%. NHP eyes were harvested 1-month post-injection. Shown is also the decrease in phosphorylation of ribosomal protein S6, which is a canonical target of S6K1. Similar to the S6K1 knockdown data, intra-animal comparison is shown to the left and comparison with several NHPs is shown to the right.

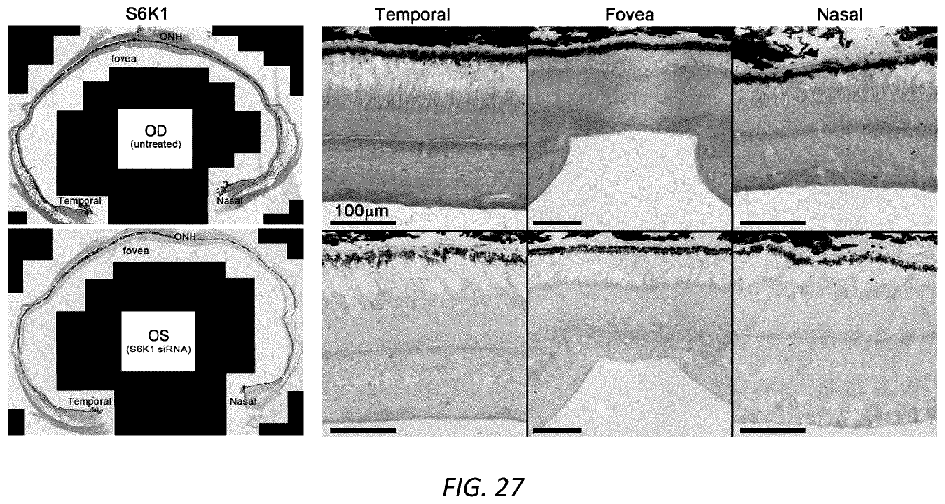

FIG. 27 displays the knockdown of S6K1 protein on retinal cross section of non-human primate (NHP) after siRNA treatment. Data is generated with the one injected eye (see also FIG. 26) and the uninjected contralateral eye. Sections were obtained from the central regions as shown for the pig in FIG. 19. Entire cross section encompassing the fovea are shown to the left in FIG. 27. Higher magnification of temporal and nasal regions as well as the fovea are shown to the right in FIG. 27. Top row of FIG. 27 shows uninjected eye and bottom row eye injected intravitreally with 225 μg of S6K1-tetramer (in 75 μL). Consistent with the western blot data presented in FIG. 26 that was generated with the superior-temporal region of the same eye, there is a clear reduction in signal for S6K1 protein expression. The reduction is also noticeable in the fovea, which contains only cones, indicating the knockdown in cones is equally efficient as in rods.

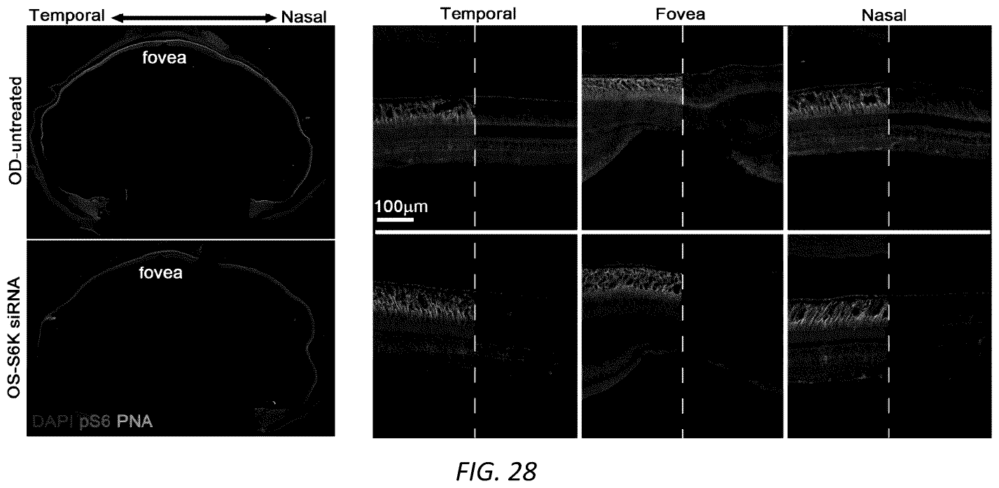

FIG. 28 shows the reduction in phosphorylated S6 protein (pS6) on retinal cross section of non-human primate (NHP) after siRNA treatment. Data in FIG. 28 is the same as shown in FIG. 27, with the exception that the staining probes for the expression of pS6 (red signal). A clear decrease of pS6 is seen across the entire retina, in particular also in photoreceptors, including foveal cones. In each panel in FIG. 28 green and blue signals have been removed from half the panel (dotted line) to better visualize the knockdown of pS6. Blue color in FIG. 28 shows nuclear DAPI while green shows cones segments marked with peanut agglutinin lectin (PNA).

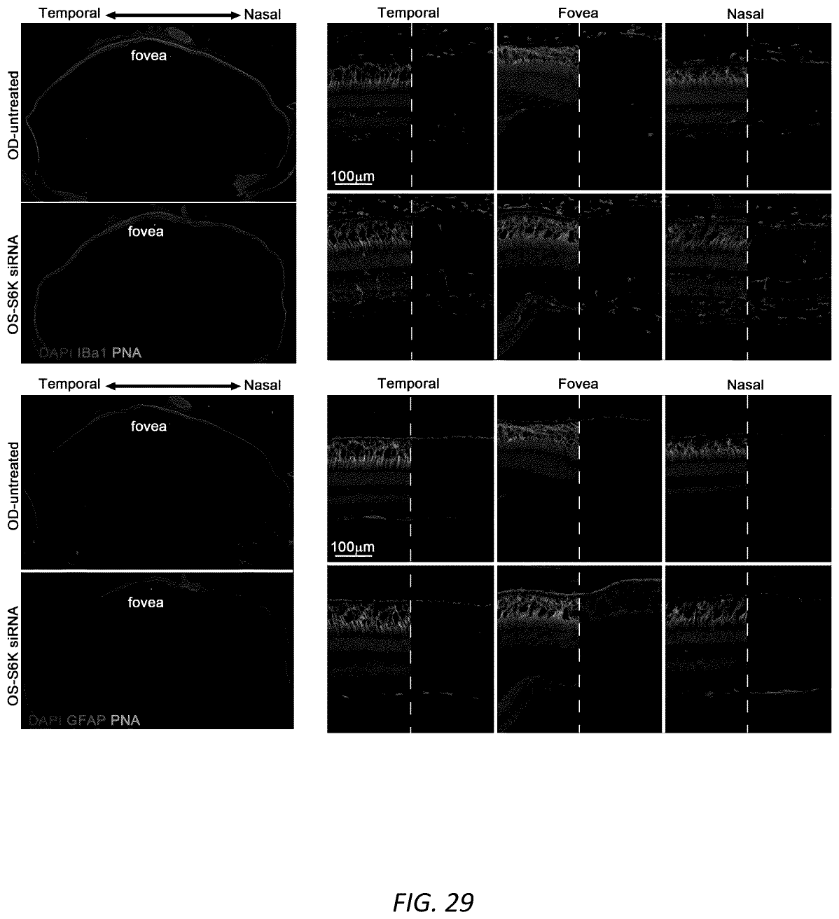

FIG. 29 shows the expression of inflammatory markers in NHP after siRNA treatment with S6K1 siRNA (75 μL, 225 μg of siRNA in tetramer configuration). Data in FIG. 29 is the same as shown in FIGS. 27 and 28, with the exception that the staining probes for the expression of Iba1 (red signal, first set) and GFAP (red signal, second set). Untreated contralateral eye is in first row of each set and the treated one in the second row. There is a slight increase in Iba1 positive cells that migrate towards the photoreceptor layer in the siRNA treated eye. However, there is no reactive gliosis as seen by GFAP staining. The staining is only slightly increased where astrocytes reside in the ganglion cell layer. There is no increase in the fovea. The data presented in FIG. 29 suggests that is no severe adverse reaction to the siRNA treatment of the knockdown of S6K1. In each panel in FIG. 29, green and blue signal have been removed from half the panel (dotted line) to better visualize the Iba1 and GFAP signal. In FIG. 29, the blue color indicates nuclear DAPI meanwhile the green color indicates cones segments marked with peanut agglutinin lectin (PNA).

Summary on S6K1-siRNA data. The Tetramer-S6K1-siRNA distributes well across the entire retina after one intravitreal delivery of 6 μg in the mouse eye. Knockdown efficiency appears slow initially but very robust over time. Therapeutically, ˜50% knockdown of S6K1 protein in photoreceptors needs to be attained, which appears to be achievable. Duplex 2 selected from the initial screen worked very efficiently in vivo, demonstrating that the S6K1 target site of SEQ ID NO: 1 is a useful target for the knockdown of S6K1. Based on the dose response curve for HTT-Tetramer in mouse, an injection of 25 μg/eye in a subset of mice will be performed and analyzed in the near future for S6K1 knockdown and for markers of age-related macular degeneration, to determine if disease progression is ameliorated. Injections in NHP confirm that knockdown is working equally efficient in a large eye, that distribution is widespread, the therapeutic range can be achieved and that there is no serious inflammatory response to the treatment. Overall, the data shows that gene knockdown in humans is feasible for the treatment of various retinal diseases.

1. A method for delivering an oligonucleotide conjugate to an eye of a subject, the method comprising administering the oligonucleotide conjugate to the subject, wherein the oligonucleotide conjugate comprises:
i) an oligonucleotide comprising a 5′ and a 3′ end and complementarity to a target nucleic acid; and
ii) a functional moiety linked to the oligonucleotide, wherein the functional moiety comprises any one of triple amine, retinoic acid, docosahexaenoic acid (DHA), docosanoic acid (DCA), α-tocopheryl succinate, or lithocholic acid (LA).

2. A method for delivering a branched oligonucleotide to an eye of a subject, the method comprising administering the branched oligonucleotide to the subject, wherein the branched oligonucleotide comprises two or more oligonucleotides, each oligonucleotide comprising a 5′ and a 3′ end and complementarity to a target nucleic acid.

3. The method of claim 2, wherein one or more of the oligonucleotides of the branched oligonucleotide further comprises a functional moiety linked to the oligonucleotide, wherein the functional moiety comprises any one of triple amine, retinoic acid, DHA, DCA, α-tocopheryl succinate, or LA.

4. The method of claim 1, wherein two DHA functional moieties are linked to the oligonucleotide.

5. The method of claim 1, wherein the oligonucleotide comprises an antisense oligonucleotide or an siRNA.

6-26. (canceled)

27. The method of claim 5, wherein the oligonucleotide comprises an antisense strand comprising at least 70% 2′-O-methyl nucleotide modifications.

28-33. (canceled)

34. The method of claim 1, wherein the functional moiety is linked to the 5′ end and/or 3′ end of the oligonucleotide.

35-42. (canceled)

43. The method of claim 5, wherein the oligonucleotide comprises a siRNA comprising a sense strand and an antisense strand, wherein nucleotides at positions 1 and 2 from the 3′ end of the sense strand, and nucleotides at positions 1 and 2 from the 5′ end of the antisense strand are connected to adjacent ribonucleotides via phosphorothioate linkages.

44-53. (canceled)

54. The method of claim 1, wherein the oligonucleotide conjugate is administered by intravitreal injection.

55. The method of claim 1, wherein the oligonucleotide conjugate is delivered to an eye cell after administration to the subject.

56-72. (canceled)

73. The method of claim 1, wherein the oligonucleotide conjugate comprises the structure:

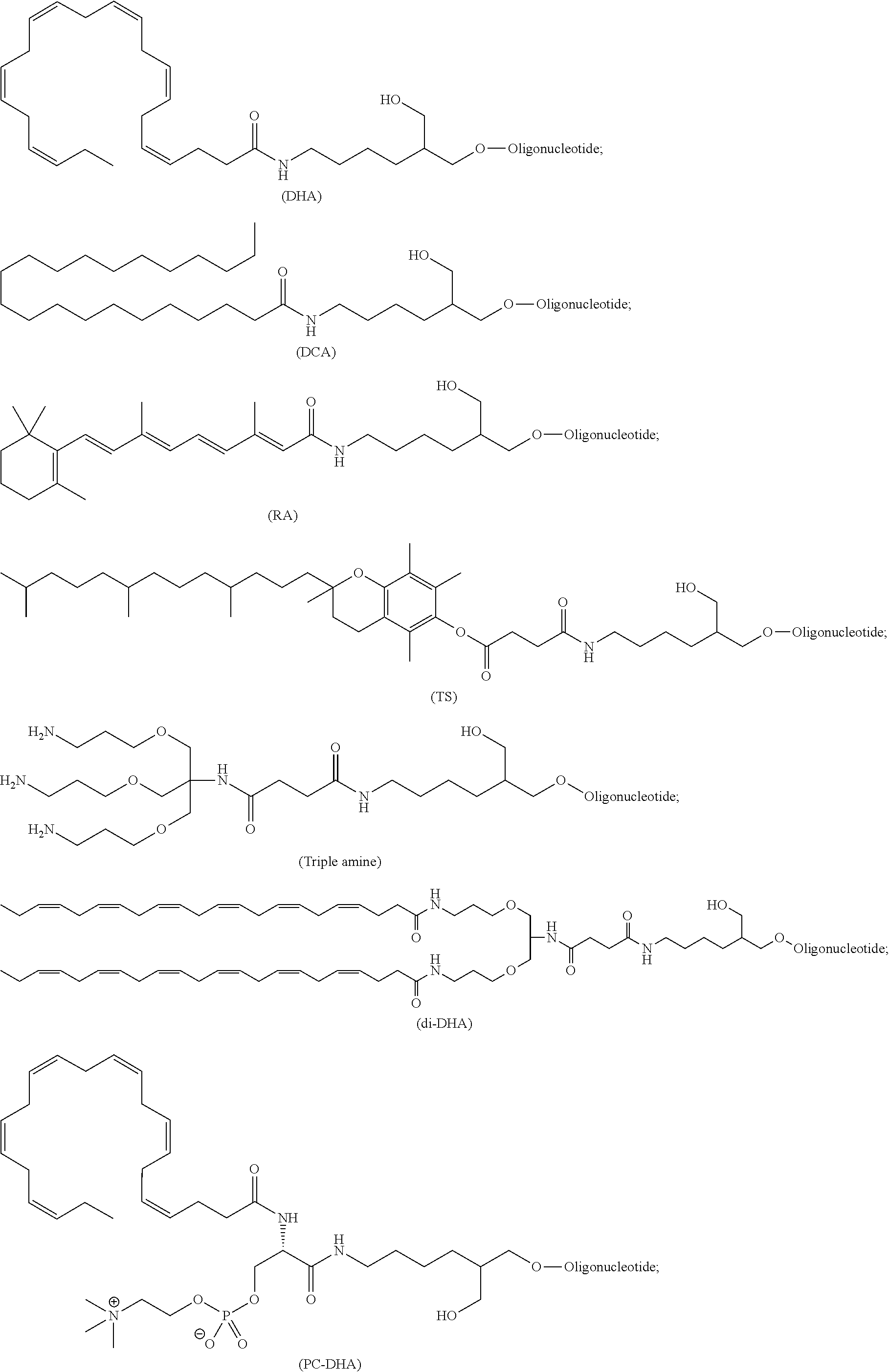

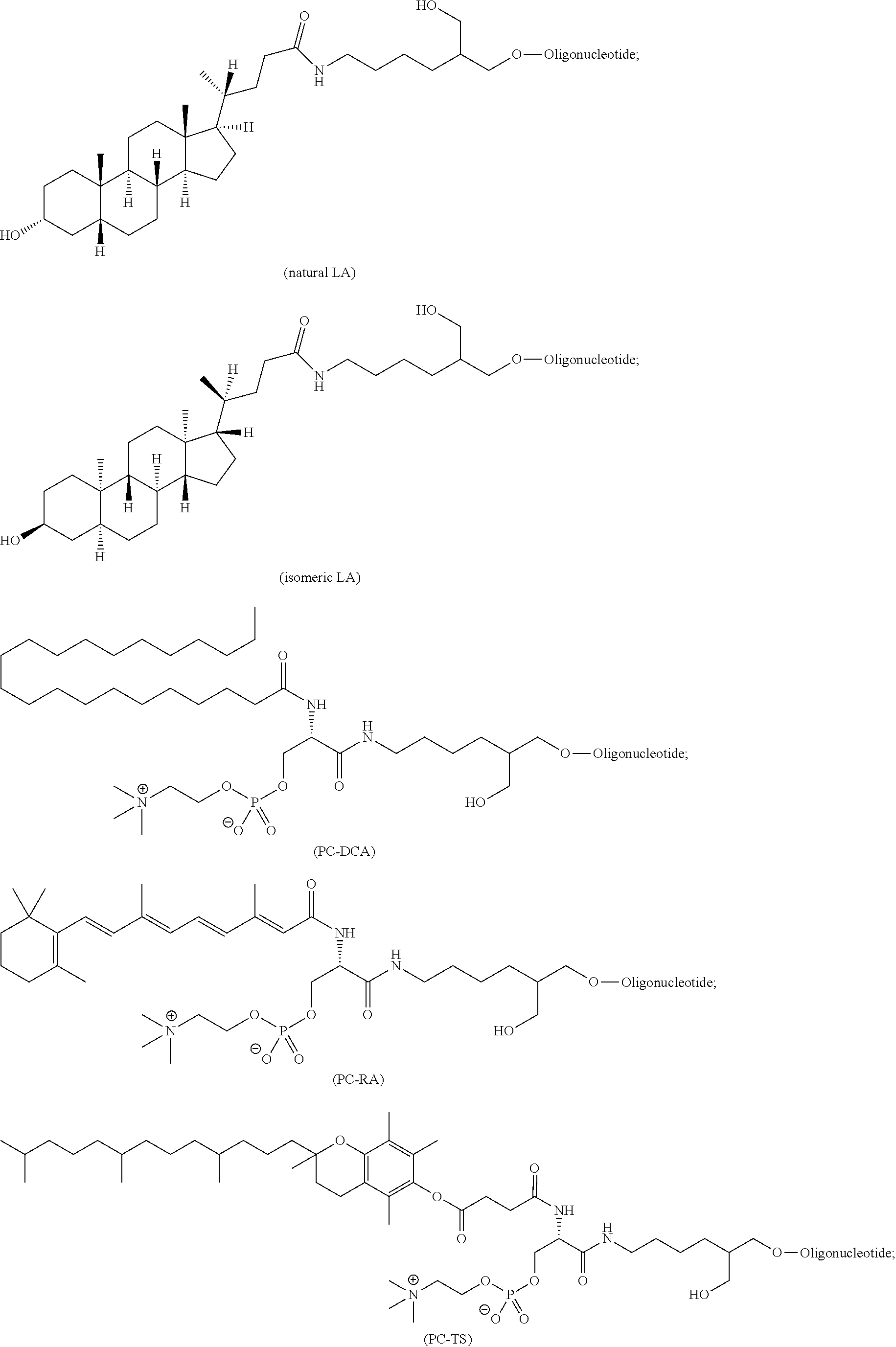

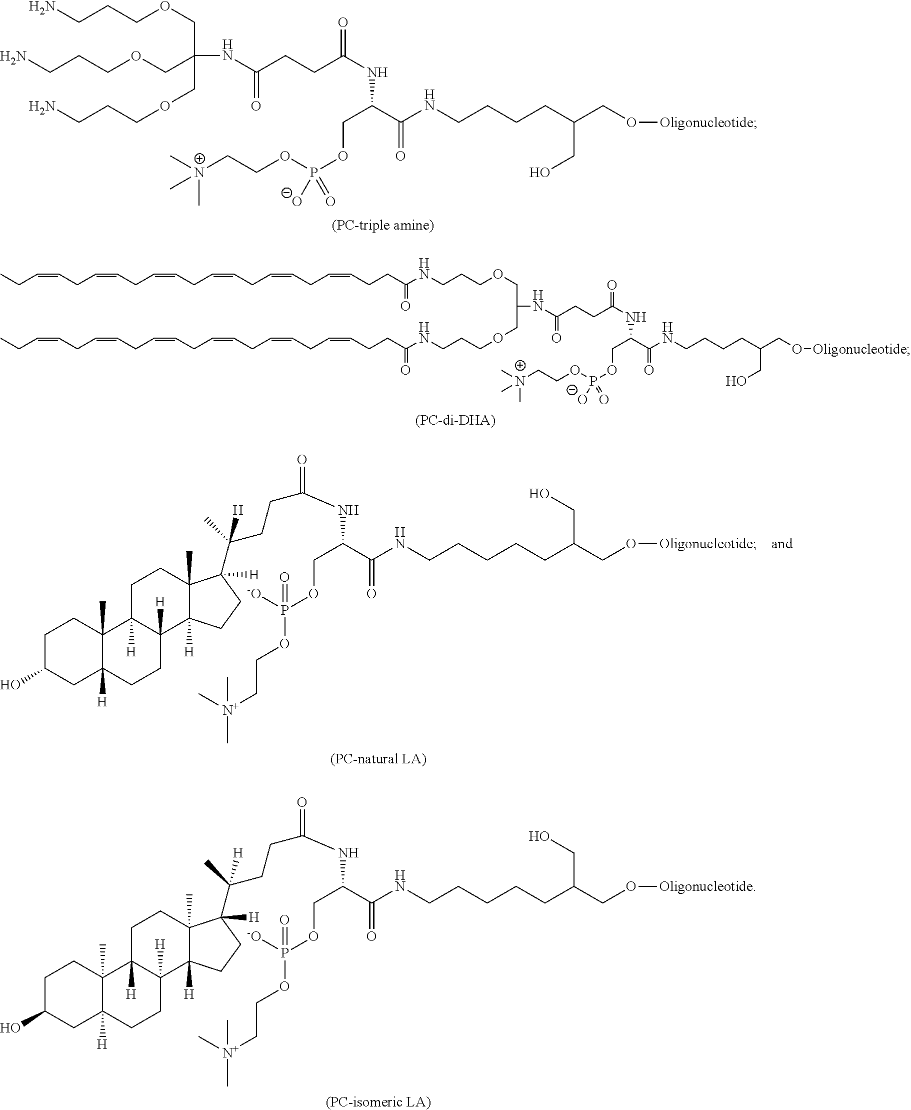

74. (canceled)

75. The method of claim 1, wherein expression of the target nucleic acid is reduced by at least 20%, at least 30%, at least 40%, or at least 50%.

76. The method of claim 1, wherein the oligonucleotide conjugate has selective affinity for a retinal protein.

77. The method of claim 1, wherein the subject comprises an eye disorder.

78-79. (canceled)

80. An oligonucleotide conjugate comprising:
i) an oligonucleotide comprising a 5′ end, a 3′ end, and complementarity to a target nucleic acid; and
ii) a di-docosahexaenoic acid (di-DHA) functional moiety linked to the oligonucleotide.

81. The oligonucleotide conjugate of claim 80, wherein the di-DHA functional moiety is phosphatidylcholine (PC) esterified di-DHA (PC-di-DHA).

82. (canceled)

83. The oligonucleotide conjugate of claim 80, wherein the oligonucleotide corresponds to an antisense oligonucleotide or a siRNA.

84-86. (canceled)

87. An oligonucleotide conjugate comprising:
i) an oligonucleotide comprising a 5′ end, a 3′ end, and complementarity to a target nucleic acid; and
ii) a triple amine functional moiety linked to the oligonucleotide.

88. The oligonucleotide conjugate of claim 87, wherein the triple amine functional moiety is phosphatidylcholine (PC) esterified triple amine (PC-triple amine).

89. (canceled)

90. The oligonucleotide conjugate of claim 87, wherein the oligonucleotide corresponds to an antisense oligonucleotide or a siRNA.

91-93. (canceled)
